(19) United States
(12) Patent Application Publication
(10) Pub. No.: US 2020/0397784 A1
(43) Pub. Date: Dec. 24, 2020

(54) FORMULATIONS OF AZAINDOLE COMPOUNDS

(71) Applicant: Janssen Pharmaceuticals, Inc., Titusville, NJ (US)

(72) Inventors: Gopal Rajan Ranga Rajan, Beerse (BE); Geert Van Der Avoort, Beerse (BE); Rudolf Josephus Dijmphna Leemans, Beerse (BE)

(21) Appl. No.: 16/851,267

(22) Filed: Apr. 17, 2020

Related U.S. Application Data
(60) Provisional application No. 62/864,055, filed on Jun. 20, 2019.
(60) Provisional application No. 62/961,241, filed on Jan. 15, 2020.

(51) Int. Cl. A61K 31/506; A61K 9/20; A61P 31/16

(52) U.S. Cl. CPC A61K 31/506; A61K 9/2027; A61K 9/2009; A61P 31/16; A61K 9/2059; A61K 9/2095; A61K 9/2013

(57) ABSTRACT

The present invention relates to pharmaceutical compositions, each comprising a multitude of granules that make up an intragranular phase of the composition, wherein the granules are produced by fluid bed granulation and comprise a HCl salt of Compound (1).xH₂O wherein x is from 0 to 3, and one or more excipients selected from a disintegrant, a binder, and a wetting agent. The pharmaceutical composition also comprises one or more excipients that make up an extragranular phase of the composition, selected from a diluent, a disintegrant, a glidant, and a lubricant. The invention also relates to processes for producing the pharmaceutical compositions of the invention. The invention further relates to uses and methods of the pharmaceutical compositions in reducing the amount of influenza viruses in a biological in vitro sample or in a subject, inhibiting the replication of influenza viruses in a biological in vitro sample or in a subject, and treating influenza in a subject.

### CROSS REFERENCE TO RELATED APPLICATIONS

This utility application claims the benefit of U.S. Provisional Application No. 62/864,055, filed on Jun. 20, 2019 and U.S. Provisional Application No. 62/961,241, filed on Jan. 15, 2020. Each of these documents is hereby incorporated by reference in its entirety.

### STATEMENT REGARDING FEDERALLY SPONSORED RESEARCH AND DEVELOPMENT

This invention was made with government support under contract number HHSO100201500014C awarded by the Office of the Assistant Secretary for Preparedness and Response, Biomedical Advanced Research and Development Authority. The government has certain rights in the invention.

### FIELD OF THE INVENTION

The present invention relates to pharmaceutical compositions, processes for producing these pharmaceutical compositions uses of these pharmaceutical compositions in treating or reducing the amount of influenza viruses in a sample or subject.

### BACKGROUND OF THE INVENTION

Influenza is primarily transmitted from person to person via large virus-laden droplets that are generated when infected persons cough or sneeze; these large droplets can then settle on the mucosal surfaces of the upper respiratory tracts of susceptible individuals who are near (e.g. within 6 feet) infected persons. Transmission might also occur through direct contact or indirect contact with respiratory secretions, such as touching surfaces contaminated with influenza virus and then touching the eyes, nose or mouth. Adults might be able to spread influenza to others from 1 day before getting symptoms to approximately 5 days after symptoms start. Young children and persons with weakened immune systems might be infectious for 10 or more days after onset of symptoms.

Influenza viruses are RNA viruses of the family Orthomyxoviridae, which comprises five genera: Influenza virus A, Influenza virus B, Influenza virus C, ISA virus and Thogoto virus.

The Influenza virus A genus has one species, influenza A virus. Wild aquatic birds are the natural hosts for a large variety of influenza A. Occasionally, viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different serotypes based on the antibody response to these viruses. The serotypes that have been confirmed in humans, ordered by the number of known human pandemic deaths, are: H1N1 (which caused Spanish influenza in 1918), H2N2 (which caused Asian Influenza in 1957), H3N2 (which caused Hong Kong Flu in 1968), H5N1 (a pandemic threat in the 2007-08 influenza season), H7N7 (which has unusual zoonotic potential), H1N2 (endemic in humans and pigs), H9N2, H7N2, H7N3 and H10N7.

The Influenza virus B genus has one species, influenza B virus. Influenza B almost exclusively infects humans and is less common than influenza A. The only other animal known to be susceptible to influenza B infection is the seal. This type of influenza mutates at a rate 2-3 times slower than type A and consequently is less genetically diverse, with only one influenza B serotype. As a result of this lack of antigenic diversity, a degree of immunity to influenza B is usually acquired at an early age. However, influenza B mutates enough that lasting immunity is not possible. This reduced rate of antigenic change, combined with its limited host range (inhibiting cross species antigenic shift), ensures that pandemics of influenza B do not occur.

The Influenza virus C genus has one species, influenza C virus, which infects humans and pigs and can cause severe illness and local epidemics. However, influenza C is less common than the other types and usually seems to cause mild disease in children.

Influenza A, B and C viruses are very similar in structure. The virus particle is 80-120 nanometers in diameter and usually roughly spherical, although filamentous forms can occur. Unusually for a virus, its genome is not a single piece of nucleic acid; instead, it contains seven or eight pieces of segmented negative-sense RNA. The Influenza A genome encodes 11 proteins: hemagglutinin (HA), neuraminidase (NA), nucleoprotein (NP), M1, M2, NS1, NS2(NEP), PA, PB1, PB1-F2 and PB2.

HA and NA are large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins have been targets for antiviral drugs. Furthermore, they are antigens to which antibodies can be raised. Influenza A viruses are classified into subtypes based on antibody responses to HA and NA, forming the basis of the H and N distinctions (vide supra) in, for example, H5N1.

Influenza produces direct costs due to lost productivity and associated medical treatment, as well as indirect costs of preventative measures. In the United States, influenza is responsible for a total cost of over $10 billion per year, while it has been estimated that a future pandemic could cause hundreds of billions of dollars in direct and indirect costs. Preventative costs are also high. Governments worldwide have spent billions of U.S. dollars preparing and planning for a potential H5N1 avian influenza pandemic, with costs associated with purchasing drugs and vaccines as well as developing disaster drills and strategies for improved border controls.

Current treatment options for influenza include vaccination, and chemotherapy or chemoprophylaxis with anti-viral medications. Vaccination against influenza with an influenza vaccine is often recommended for high-risk groups, such as children and the elderly, or in people that have asthma, diabetes, or heart disease. However, it is possible to get vaccinated and still get influenza. The vaccine is reformulated each season for a few specific influenza strains but cannot possibly include all the strains actively infecting people in the world for that season. It may take six months for the manufacturers to formulate and produce the millions of doses required to deal with the seasonal epidemics; occasionally, a new or overlooked strain becomes prominent during that time and infects people although they have been vaccinated (as by the H3N2 Fujian flu in the 2003-2004 influenza season). It is also possible to get infected just before vaccination and get sick with the very strain that the vaccine is supposed to prevent, as the vaccine may take several weeks to become effective.

Further, the effectiveness of these influenza vaccines is variable. Due to the high mutation rate of the virus, a particular influenza vaccine usually confers protection for no more than a few years. A vaccine formulated for one year may be ineffective in the following year, since the influenza virus changes rapidly over time, and different strains become dominant.

Also, because of the absence of RNA proofreading enzymes, the RNA-dependent RNA polymerase of influenza vRNA makes a single nucleotide insertion error roughly every 10 thousand nucleotides, which is the approximate length of the influenza vRNA. Hence, nearly every newly-manufactured influenza virus is a mutant-antigenic drift. The separation of the genome into eight separate segments of vRNA allows mixing or reassortment of vRNAs if more than one viral line has infected a single cell. The resulting rapid change in viral genetics produces antigenic shifts and allows the virus to infect new host species and quickly overcome protective immunity.

Antiviral drugs can also be used to treat influenza, with neuraminidase inhibitors being particularly effective, but viruses can develop resistance to the standard antiviral drugs.

Thus, there is still a need for drugs for treating influenza infections, such as for drugs with expanded treatment window, and/or reduced sensitivity to viral titer.

### SUMMARY OF THE INVENTION

The present invention generally relates to pharmaceutical compositions that comprise Compound (1), or a salt or salt form thereof, methods of preparing such pharmaceutical compositions, and methods of using such pharmaceutical compositions for treating influenza employing such pharmaceutical compositions. Compound (1) is represented by the following structural formula:

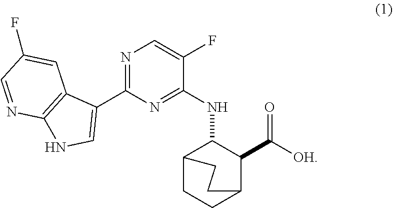

The present invention provides a pharmaceutical composition comprising (a) a plurality of granules forming an intragranular phase of the composition, wherein the granules are produced by fluid bed granulation and comprise (i) a crystalline HCl salt of Compound (1).xH₂O (x=0, 0.5 (or ½), 1, 2, or 3) wherein Compound (1) is represented by the following structural formula:

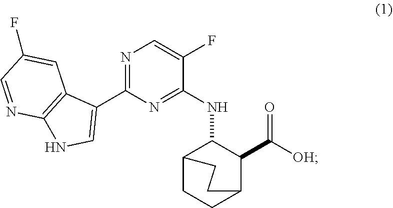

and
(ii) one or more excipients selected from a disintegrant, a binder, and a wetting agent; and (b) one or more excipients forming an extragranular phase of the composition, selected from a diluent, a disintegrant, a glidant, and a lubricant, wherein the HCl salt of Compound (1).½H₂O has a concentration of 5 wt % to 95 wt % by weight of the composition, and the one or more excipients has a concentration of 5 wt % to 95 wt % by weight of the composition.

In some embodiments, the crystalline HCl salt of Compound (1).xH₂O is crystalline HCl salt of Compound (1).½H₂O (i.e., the crystalline HCl salt of Compound (1) hemihydrate). For instance, the crystalline HCl salt of Compound (1).½H₂O is Compound (1) crystalline HCl hemihydrate salt Form A.

In some embodiments, the composition comprises 10 wt % to 80 wt % of a diluent by weight of the pharmaceutical composition. In some examples, the diluent comprises microcrystalline cellulose, starch, silica, or any combination thereof.

In some embodiments, the composition comprises 1 wt % to 10 wt % of a disintegrant by the weight of the pharmaceutical composition. In some examples, the disintegrant comprises croscarmellose, crospovidone, or any combination thereof. For instance, the disintegrant comprises crospovidone.

In some embodiments, the composition comprises 0.1 wt % to 10 wt % of a binder by weight of the pharmaceutical composition. In some embodiments, the composition comprises 0.1 wt % to 5 wt % of a binder by the weight of the pharmaceutical composition. In some examples, the binder comprises hydroxypropyl methylcellulose.

In some embodiments, the composition comprises 0.5 wt % to 10 wt % of a lubricant by weight of the pharmaceutical composition. In some embodiments, the composition comprises 0.5 wt % to 6 wt % of a lubricant by the weight of the pharmaceutical composition. In some examples, the lubricant comprises sodium stearyl fumarate, magnesium stearate, or any combination thereof.

In some embodiments, the composition comprises 0.1 wt % to 1.0 wt % of a wetting agent by the weight of the pharmaceutical composition. In some examples, the wetting agent comprises polysorbate 20.

In some embodiments, the composition comprises 0.1 wt % to 10 wt % of a glidant by the weight of the pharmaceutical composition. In some embodiments, the composition comprises 1 wt % to 10 wt % of a glidant by the weight of the pharmaceutical composition. In some examples, the glidant comprises silicon dioxide.

In some embodiments, the pharmaceutical composition comprises (a) 20 wt % to 80 wt % (e.g., 30 wt % to 70 wt %, 40 wt % to 70 wt %, or 49 wt % to 54 wt %) of Compound (1) HCl hemihydrate crystalline salt Form A by weight of the pharmaceutical composition; (b) 1 wt % to 10 wt % (e.g., 5 wt % to 8 wt %, 6 wt % to 8 wt %, or 6.5 wt % to 7.5 wt %) of the disintegrant by weight of the pharmaceutical composition; (c) 1 wt % to 10 wt % (e.g., 1 wt % to 5 wt %, 1 wt % to 3 wt %, 1.54 wt % to 1.70 wt %, or 1.85 wt % to 1.95 wt %) of the binder by weight of the pharmaceutical composition; (d) 0.1 wt % to 1.0 wt % (e.g., 0.1 wt % to 0.6 wt %, 0.15 wt % to 0.55 wt %, 0.20 wt % to 0.3 wt %, or 0.45 wt % to 0.55 wt %) of the wetting agent by the weight of the pharmaceutical composition; (e) 0.1 wt % to 5.0 wt % (e.g., 0.5 wt % to 2.0 wt %, 0.5 wt % to 1.5 wt %, or 0.95 wt % to 1.05 wt %) of the glidant by weight of the pharmaceutical composition; (f) 1 wt % to 10 wt % (e.g., 1.5 wt % to 6 wt %, 1.5 wt % to 5.5 wt %, 2.85 wt % to 3.15 wt %, 1.85 wt % to 2.15 wt %, or 4.85 wt % to 5.15 wt %) of the lubricant by weight of the pharmaceutical composition; and (g) 20 wt % to 80 wt % (e.g., 20 wt % to 45 wt %, 25 wt % to 40 wt %, 34 wt % to 38 wt %, or 25.0 wt % to 27.0 wt %) of the diluent by weight of the pharmaceutical composition.

In some embodiments, the intragranular phase of the composition comprises (a) 49 wt % to 54 wt % of Compound (1) HCl hemihydrate crystalline salt Form A by weight of the pharmaceutical composition; (b) 1.45 wt % to 1.62 wt % of the disintegrant by weight of the pharmaceutical composition; (c) 1.54 wt % to 1.70 wt % of the binder by weight of the pharmaceutical composition; and (d) 0.21 wt % to 0.25 wt % of the wetting agent by weight of the pharmaceutical composition.

In some embodiments, the extragranular phase of the composition comprises (a) 5.0 wt % to 6.0 wt % of the disintegrant by weight of the pharmaceutical composition; (b) 0.95 wt % to 1.05 wt % of the glidant by weight of the pharmaceutical composition; (c) 2.85 wt % to 3.15 wt % of the lubricant by weight of the pharmaceutical composition; and (d) 34 wt % to 38 wt % of the diluent by weight of the pharmaceutical composition.

In some embodiments, the Compound (1) HCl hemihydrate crystalline salt Form A is in a micronized state in the pharmaceutical composition.

In some embodiments, the Compound (1) HCl hemihydrate crystalline salt Form A is present in an amount of about 51.42 wt % by weight of the pharmaceutical composition.

In some embodiments, the disintegrant in the intragranular phase is crospovidone. In some examples, the disintegrant in the intragranular phase is present in an amount of about 1.54 wt % by weight of the pharmaceutical composition.

In some embodiments, the binder is hydroxypropyl methylcellulose. In some examples, the binder is hydroxypropyl methylcellulose having a viscosity of about 15 mPa·s. And, in some examples, the binder is present in an amount of about 1.54 wt % by weight of the pharmaceutical composition. In one embodiment, the binder is present in an amount of about 1.62 wt % by weight of the pharmaceutical composition

In some embodiments, the wetting agent is a polysorbate. In some examples, the wetting agent is polysorbate 20. In some examples, the wetting agent is present in an amount of about 0.23 wt % by weight of the pharmaceutical composition.

In some embodiments, the glidant is colloidal anhydrous silica. In some examples, the glidant is present in an amount of about 1.00 wt % by weight of the pharmaceutical composition.

In some embodiments, the disintegrant in the extragranular phase is crospovidone. In some examples, the disintegrant in the extragranular phase is present in an amount of about 5.46 wt % by weight of the pharmaceutical composition.

In some embodiments, the lubricant is sodium stearyl fumarate. In some examples, the lubricant is present in an amount of about 3.00 wt % by weight of the pharmaceutical composition.

In some embodiments, the diluent comprises silicified microcrystalline cellulose, microcrystalline cellulose, starch, or any combination thereof. In some examples, the starch is partially or fully pregelatinized maize starch. In some examples, the diluent is present in an amount of about 35.74 wt % by weight of the pharmaceutical composition. In some examples, the diluent comprises silicified microcrystalline cellulose. For instance, the silicified microcrystalline cellulose is present in an amount of about 25.74 wt % by weight of the pharmaceutical composition. In other examples, the diluent comprises silicified microcrystalline cellulose and starch. For instance, the silicified microcrystalline cellulose is present in an amount of about 25.74 wt % by weight of the pharmaceutical composition and the starch is present in an amount of about 10.00 wt % by weight of the pharmaceutical composition. And, in other examples, the diluent comprises silicified microcrystalline cellulose and microcrystalline cellulose. For instance, the silicified microcrystalline cellulose is present in an amount of about 25.74 wt % by weight of the pharmaceutical composition, and the microcrystalline cellulose is present in an amount of about 10.00 wt % by weight of the pharmaceutical composition. In other examples, the diluent comprises silicified microcrystalline cellulose, microcrystalline cellulose, and starch. For instance, the silicified microcrystalline cellulose is present in an amount of about 25.74 wt % by weight of the pharmaceutical composition, the microcrystalline cellulose is present in an amount of about 5.00 wt % by weight of the pharmaceutical composition, and the starch is present in an amount of about 5.00 wt % by weight of the pharmaceutical composition.

In some embodiments, the pharmaceutical composition comprises (a) 57.50 wt % to 64.00 wt % of Compound (1) HCl hemihydrate crystalline salt Form A by weight of the pharmaceutical composition; (b) 6.5 wt % to 7.5 wt % of the disintegrant by weight of the pharmaceutical composition; (c) 1.80 wt % to 2.10 wt % of the binder by weight of the pharmaceutical composition; (d) 0.25 wt % to 0.30 wt % of the wetting agent by weight of the pharmaceutical composition; (e) 0.95 wt % to 1.05 wt % of the glidant by the weight of the pharmaceutical composition; (f) 2.85 wt % to 3.15 wt % of the lubricant by weight of the pharmaceutical composition; and (g) 24.5 wt % to 27.5 wt % of the diluent by weight of the pharmaceutical composition.

In some embodiments, the intragranular phase of the composition comprises (a) 57.50 wt % to 64.00 wt % (e.g., 50 wt % to 53 wt %) of Compound (1) HCl hemihydrate crystalline salt Form A by weight of the pharmaceutical composition; (b) 1.70 wt % to 1.95 wt % (e.g., 1.42 wt % to 1.58 wt %) of the disintegrant by weight of the pharmaceutical composition; (c) 1.80 wt % to 2.10 wt % (e.g., 1.80 wt % to 2.00 wt %) of the binder by weight of the pharmaceutical composition; and (d) 0.25 wt % to 0.30 wt % (e.g., 0.47 wt % to 0.53 wt %) of the wetting agent by weight of the pharmaceutical composition.

In some embodiments, the Compound (1) HCl hemihydrate crystalline salt Form A in the intragranular phase is in a micronized state in the pharmaceutical composition. In other examples, the Compound (1) HCl hemihydrate crystalline salt Form A is present in an amount of about 60.76 wt % by weight of the pharmaceutical composition.

In some embodiments, the disintegrant in the intragranular phase is crospovidone. For example, the disintegrant in the intragranular phase is present in an amount of about 1.82 wt % by weight of the pharmaceutical composition.

In some embodiments, the binder in the intragranular phase is hydroxypropyl methylcellulose. For example, the binder is hydroxypropyl methylcellulose having a viscosity of about 15 mPa·s. In other examples, the binder is present in the intragranular phase in an amount of about 1.91 wt % by weight of the pharmaceutical composition.

In some embodiments, the wetting agent is a polysorbate. For example, the wetting agent is polysorbate 20. In other examples, the wetting agent is present in an amount of about 0.27 wt % by weight of the pharmaceutical composition.

In some embodiments, the extragranular phase of the composition comprises (a) 4.5 wt % to 5.7 wt % (e.g., 5.25 wt % to 5.75 wt %) of the disintegrant by weight of the pharmaceutical composition; (b) 0.95 wt % to 1.05 wt % (e.g., 0.95 wt % to 1.05 wt %) of the glidant by weight of the pharmaceutical composition; (c) 2.9 wt % to 3.1 wt % (e.g., 4.75 wt % to 5.25 wt %) of the lubricant by weight of the pharmaceutical composition; and (d) 24.5 wt % to 27.5 wt % (e.g., 31.50 wt % to 35.00 wt %) of the diluent by weight of the pharmaceutical composition.

In some embodiments, the glidant in the extragranular phase is colloidal anhydrous silica. For example, the glidant is present in the extragranular phase in an amount of about 1.00 wt % by weight of the pharmaceutical composition.

In some embodiments, the disintegrant in the extragranular phase is crospovidone. In some examples, the disintegrant in the extragranular phase is present in an amount of about 5.18 wt % by weight of the pharmaceutical composition.

In some embodiments, the lubricant in the extragranular phase is sodium stearyl fumarate. In some examples, the lubricant is present in an amount of about 3.00 wt % by weight of the pharmaceutical composition.

In some embodiments, the diluent of the extragranular phase comprises silicified microcrystalline cellulose, microcrystalline cellulose, starch, or any combination thereof. In some examples, wherein the starch is partially or fully pregelatinized maize starch. In some examples, the diluent is present in an amount of about 26.05 wt % by weight of the pharmaceutical composition. In some examples, the diluent comprises silicified microcrystalline cellulose. In some examples, the silicified microcrystalline cellulose is present in an amount of about 18.55 wt % by weight of the pharmaceutical composition. In some examples, the diluent comprises silicified microcrystalline cellulose and starch. In some examples, the silicified microcrystalline cellulose is present in an amount of about 18.55 wt % by weight of the pharmaceutical composition, and the starch is present in an amount of about 7.50 wt % by weight of the pharmaceutical composition. In some examples, the diluent comprises silicified microcrystalline cellulose and microcrystalline cellulose. In some examples, the silicified microcrystalline cellulose is present in an amount of about 18.55 wt % by weight of the pharmaceutical composition, and the microcrystalline cellulose is present in an amount of about 7.5 wt % by weight of the pharmaceutical composition. In other examples, the diluent comprises silicified microcrystalline cellulose, microcrystalline cellulose, and starch. In some examples, the silicified microcrystalline cellulose is present in an amount of about 18.55 wt % by weight of the pharmaceutical composition, the microcrystalline cellulose is present in an amount of about 3.75 wt % by weight of the pharmaceutical composition, and the starch is present in an amount of about 3.75 wt % by weight of the pharmaceutical composition.

In some embodiments, the Compound (1) HCl hemihydrate crystalline salt Form A is in a micronized state in the pharmaceutical composition. In some examples, the Compound (1) HCl hemihydrate crystalline salt Form A is present in an amount of about 51.42 wt % by weight of the pharmaceutical composition.

In some embodiments, the disintegrant in the intragranular phase is crospovidone. In some examples, the disintegrant in the intragranular phase is present in an amount of about 1.50 wt % by weight of the pharmaceutical composition.

In some embodiments, the binder is hydroxypropyl methylcellulose. In some examples, the binder is hydroxypropyl methylcellulose having a viscosity of about 15 mPa·s. In some examples, the binder is present in an amount of about 1.90 wt % by weight of the pharmaceutical composition.

In some embodiments, the wetting agent is a polysorbate. For example, the wetting agent is polysorbate 20. In some examples, the wetting agent is present in an amount of about 0.50 wt % by weight of the pharmaceutical composition.

In some embodiments, the glidant is colloidal anhydrous silica. In some examples, the glidant is present in an amount of about 1.00 wt % by weight of the pharmaceutical composition.

In some embodiments, the disintegrant in the extragranular phase is crospovidone. In some examples, the disintegrant in the extragranular phase is present in an amount of about 5.50 wt % by weight of the pharmaceutical composition.

In some embodiments, the lubricant is sodium stearyl fumarate. In some examples, the lubricant is present in an amount of about 5.00 wt % by weight of the pharmaceutical composition.

In some embodiments, the diluent comprises silicified microcrystalline cellulose, microcrystalline cellulose, starch, or any combination thereof. In some examples, the starch is partially or fully pregelatinized maize starch. In some examples, the diluent is present in an amount of about 33.18 wt % by weight of the pharmaceutical composition. In some examples, the diluent comprises silicified microcrystalline cellulose. In some examples, the silicified microcrystalline cellulose is present in an amount of about 23.18 wt % by weight of the pharmaceutical composition. In some examples, the diluent comprises silicified microcrystalline cellulose, microcrystalline cellulose, and starch. In some examples, the silicified microcrystalline cellulose is present in an amount of about 23.18 wt % by weight of the pharmaceutical composition, the microcrystalline cellulose is present in an amount of about 5.00 wt % by weight of the pharmaceutical composition, and the starch is present in an amount of about 5.00 wt % by weight of the pharmaceutical composition.

In some embodiments, the pharmaceutical composition is in the form of a tablet. In a further embodiment, the pharmaceutical composition is in the form of a tablet, wherein the total tablet weight is from about 645 mg to about 675 mg, or the total tablet weight is from about 1090 mg to about 1140 mg, or the total tablet weight is from about 1290 mg to about 1345 mg. In some examples, the tablet further comprises a film coating. And, in some examples, the film coating comprises a polymer, plasticizer and pigment. In some examples, the film coating comprises a polymer, plasticizer, an anti-tacking agent, and pigment. In one embodiment, the anti-tacking agent is talc. In one embodiment, the pigment is titanium dioxide. For instance, the film coating comprises a white pigment (e.g., Opadry® II White 85F18422). In other instances, the film coating comprises a yellow pigment (e.g., Opadry® II 85F92450).

The present invention also provides a pharmaceutical composition comprising tablet, wherein the tablet comprises (a) a plurality of granules that form an intragranular phase of the composition, wherein the granules are produced by fluid bed granulation and comprise (i) about 51.42 wt % of Compound (1) HCl hemihydrate crystalline salt Form A by weight of the pharmaceutical composition, wherein Compound (1) is represented by the following structural formula:

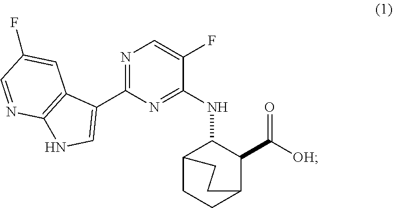

(ii) about 1.54 wt % of crospovidone by weight of the pharmaceutical composition; (iii) about 1.62 wt % of hydroxypropyl methylcellulose having a viscosity of about 15 mPa·s, by weight of the pharmaceutical composition; and (iv) about 0.23 wt % of polysorbate 20 by weight of the pharmaceutical composition; and (b) one or more excipients that form an extragranular phase of the composition, wherein the extragranular phase of the composition comprises (i) about 5.46 wt % of crospovidone by weight of the pharmaceutical composition; (ii) about 1.00 wt % of colloidal anhydrous silica by weight of the pharmaceutical composition; (iii) about 3.00 wt % of sodium stearyl fumarate by weight of the pharmaceutical composition; (iv) about 25.74 wt % of silicified microcrystalline cellulose by weight of the pharmaceutical composition; (v) about 5.00 wt % of microcrystalline cellulose by weight of the pharmaceutical composition; and (vi) about 5.00 wt % of starch by weight of the pharmaceutical composition.

In some embodiments, each tablet comprises an intragranular phase and an extragranular phase, the intragranular phase of the composition comprising (a) about 668.40 mg of Compound (1) HCl hemihydrate crystalline salt Form A; (b) about 20.00 mg of crospovidone; (c) about 21.00 mg of hydroxypropyl methylcellulose having a viscosity of about 15 mPa·s; and (d) about 3.00 of polysorbate 20; and the extragranular phase of the composition comprising (a) about 71.00 mg of crospovidone; (b) about 13.00 mg of colloidal anhydrous silica; (c) about 39.00 mg of sodium stearyl fumarate; (d) about 334.60 mg of silicified microcrystalline cellulose; (e) about 65.00 mg of microcrystalline cellulose; and (f) about 65.00 mg of starch.

In some embodiments, each tablet comprises an intragranular phase and an extragranular phase, the intragranular phase of the composition comprising (a) about 334.20 mg of Compound (1) HCl hemihydrate crystalline salt Form A; (b) about 10.00 mg of crospovidone; (c) about 10.50 mg of hydroxypropyl methylcellulose having a viscosity of about 15 mPa·s; and (d) about 1.50 of polysorbate 20; and the extragranular phase of the composition comprising (a) about 35.50 mg of crospovidone; (b) about 6.50 mg of colloidal anhydrous silica; (c) about 19.50 mg of sodium stearyl fumarate; (d) about 167.30 mg of silicified microcrystalline cellulose; (e) about 32.50 mg of microcrystalline cellulose; and (f) about 32.50 mg of starch.

In some embodiments, a pharmaceutical composition comprising a tablet, wherein the tablet comprises (a) a plurality of granules forming an intragranular phase of the composition, wherein the granules are produced by fluid bed granulation and comprise (i) about 51.42 wt % of Compound (1) HCl hemihydrate crystalline salt Form A by weight of the pharmaceutical composition, wherein Compound (1) is represented by the following structural formula:

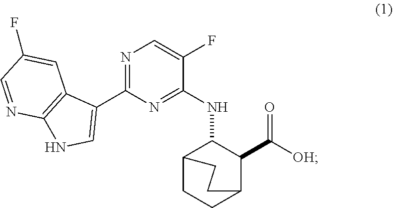

(ii) about 1.50 wt % of crospovidone by weight of the pharmaceutical composition; (iii) about 1.90 wt % of hydroxypropyl methylcellulose having a viscosity of about 15 mPa·s, by weight of the pharmaceutical composition; and (iv) about 0.50 wt % of polysorbate 20 by weight of the pharmaceutical composition; and (b) one or more excipients forming an extragranular phase of the composition, wherein the extragranular phase of the composition comprises (i) about 5.50 wt % of crospovidone by weight of the pharmaceutical composition; (ii) about 1.00 wt % of colloidal anhydrous silica by weight of the pharmaceutical composition; (iii) about 5.00 wt % of sodium stearyl fumarate by weight of the pharmaceutical composition; (iv) about 23.18 wt % of silicified microcrystalline cellulose by weight of the pharmaceutical composition; (v) about 5.00 wt % of microcrystalline cellulose by weight of the pharmaceutical composition; and (vi) about 5.00 wt % of starch by weight of the pharmaceutical composition.

In some embodiments, each tablet comprises an intragranular phase and an extragranular phase, (a) the intragranular phase of the composition comprising (i) about 334.20 mg of Compound (1) HCl hemihydrate crystalline salt Form A; (ii) about 9.75 mg of crospovidone; (iii) about 12.35 mg of hydroxypropyl methylcellulose having a viscosity of about 15 mPa·s; and (iv) about 3.25 mg of polysorbate 20; (b) and the extragranular phase of the composition comprising (i) about 35.75 mg of crospovidone; (ii) about 6.5 mg of colloidal anhydrous silica; (iii) about 32.50 mg of sodium stearyl fumarate; (iv) about 150.70 mg of silicified microcrystalline cellulose; (v) about 32.50 mg of microcrystalline cellulose; and (vi) about 32.50 mg of starch.

The present invention also provides a process for producing a pharmaceutical composition comprising (a) mixing a binder and a wetting agent in water to form a substantially clear binder solution; (b) mixing Compound (1) HCl hemihydrate crystalline salt Form A and a disintegrant under heating conditions in a fluid bed granulizer to form a substantially homogenous mixture; (c) spraying the binder solution onto the homogenous mixture under fluidizing conditions to form a plurality wet granules; (d) drying the wet granules under fluidizing conditions to form dry granules; (e) mixing the dry granules and a glidant to form a substantially homogenous second mixture; (f) mixing a diluent, a second disintegrant, and the homogenous second mixture to form a substantially homogenous third mixture; (g) mixing a lubricant and the homogenous third mixture to form a substantially homogenous fourth mixture; and (h) compressing the homogenous fourth mixture into tablets using a tablet press.

In some implementations, the binder is hydroxypropyl methylcellulose (HPMC), and/or the wetting agent is polysorbate 20.

In some implementations, the first disintegrant is crospovidone.

In some implementations, the glidant is colloidal anhydrous silica.

In some implementations, the diluent comprises silicified microcrystalline cellulose, microcrystalline cellulose, pregelatinized starch, or any combination thereof. For example, the diluent comprises silicified microcrystalline cellulose and pregelatinized starch. In other examples, the diluent comprises silicified microcrystalline cellulose and microcrystalline cellulose.

In some implementations, the lubricant is sodium stearyl fumarate.

Some implementations further comprise coating the tablet with a coating material, wherein the coating material comprises a polymer, plasticizer and pigment.

The present invention also provides a method of reducing the amount of influenza viruses in a biological in vitro sample or in a subject, comprising administering to the sample or subject an effective amount of a pharmaceutical composition as described herein.

The present invention also provides a method of inhibiting the replication of influenza viruses in a biological in vitro sample or in a subject, comprising administering to the sample or subject an effective amount of a pharmaceutical composition as described herein.

The present invention also provides a method of treating influenza in a subject, comprising administering to the subject a therapeutically effective amount of a pharmaceutical composition as described herein.

Some implementations further comprise co-administering one or more additional therapeutic agents to the sample or subject. In some examples, the additional therapeutic agents comprise an anti-virus drug. In some instances, the anti-virus drug comprises a neuraminidase inhibitor (e.g., oseltamivir, zanamivir, or any combination thereof). In other instances, the anti-virus drug comprises a polymerase inhibitor (e.g., favipiravir).

In some implementations, the influenza viruses are influenza A viruses.

The present invention also provides a dosage regimen comprising administering to a subject an effective amount of a pharmaceutical composition as described herein in a dosage amount of 100 mg to 1,600 mg of Compound (1) HCl hemihydrate crystalline salt Form A.

In some embodiments, the dosage amount of Compound (1) HCl hemihydrate crystalline salt Form A is from 400 mg to 1000 mg. In some examples, the dosage amount of Compound (1) HCl hemihydrate crystalline salt Form A is from 600 mg to 700 mg. And, in some examples, the dosage amount of Compound (1) HCl hemihydrate crystalline salt Form A is about 668.4 mg.

In some embodiments, the dosage amount of Compound (1) HCl hemihydrate crystalline salt Form A is from 200 mg to 500 mg. In some examples, the dosage amount of Compound (1) HCl hemihydrate crystalline salt Form A is from 300 mg to 400 mg. In other examples, the dosage amount of Compound (1) HCl hemihydrate crystalline salt Form A is about 334.20 mg.

### BRIEF DESCRIPTION OF DRAWINGS

FIG. 1 is an X-ray powder diffraction (XRPD) pattern of Compound (1) HCl hemihydrate crystalline salt Form A.

FIG. 2 is a ¹³C solid state nuclear magnetic spectroscopy (¹³C SSNMR) spectrum of Compound (1) HCl hemihydrate Salt Form A.

FIG. 3 is a graph showing AUC viral shedding for 1200 mg/600 mg of Compound (1) HCl hemihydrate salt Form A dose group in a live, attenuated influenza challenge model in humans.

FIG. 4A is a graph showing the dissolution profiles of selected formulations prior to stressed conditions, wherein the profiles were obtained by the QC-2 dissolution method.

FIG. 4B is a graph showing the dissolution profiles of selected formulations prior to stressed conditions, wherein the profiles were obtained by the QC-3 dissolution method.

FIG. 5 is a graph showing a comparison of the dissolution of 3 different batches of the tablet composition 7 to the Phase 2b formulation using the Physiology based dissolution method (PBDT).

FIG. 6 is a graph comparing the dissolution profiles of Tablet Compositions 7 and 8 using the QC-4 dissolution method.

FIG. 7 is a graph comparing the dissolution profiles of Tablet Compositions 7 and 9 using the QC-4 dissolution method.

### DETAILED DESCRIPTION OF THE INVENTION

The present invention provides pharmaceutical compositions that comprise crystalline Compound (1) HCl hemihydrate (e.g., Compound (1) HCl hemihydrate crystalline salt Form A), methods of preparing such pharmaceutical compositions, methods of treating influenza, methods of reducing the amount of influenza viruses, and methods of inhibiting the replication of influenza viruses employing such pharmaceutical compositions.

### I. DEFINITIONS

As used herein, an “excipient” is an inactive ingredient in a pharmaceutical composition. Examples of excipients include diluents, wetting agents (e.g., surfactants), binders, glidants, lubricants, disintegrants, and the like.

As used herein, a “disintegrant agent” or “disintegrant” is an excipient that hydrates a pharmaceutical composition and aids in tablet dispersion. Examples of disintegrant agents include croscarmellose sodium, crospovidone (i.e., cross-linked polyvinyl N-pyrrolidone), sodium starch glycolate, or any combination thereof.

As used herein, a “diluent” or “filler” is an excipient that adds bulkiness to a pharmaceutical composition. Examples of diluents include lactose, sorbitol, celluloses, calcium phosphates, starches, sugars (e.g., mannitol, sucrose, or the like) or any combination thereof.

As used herein, a “wetting agent” or a “surfactant” is an excipient that imparts pharmaceutical compositions with enhanced solubility and/or wetability. Examples of wetting agents include sodium lauryl sulfate (SLS), sodium stearyl fumarate (SSF), polyoxyethylene 20 sorbitan mono-oleate (i.e., polysorbate 20) (e.g., Tween™ or Tween 20), or any combination thereof.

As used herein, a “binder” is an excipient that imparts a pharmaceutical composition with enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein, a “glidant” is an excipient that imparts pharmaceutical composition with enhanced flow properties. Examples of glidants include colloidal silica and/or talc.

As used herein, a “colorant” is an excipient that imparts a pharmaceutical composition with a desired color. Examples of colorants include commercially available pigments such as FD&C Blue #1 Aluminum Lake, FD&C Blue #2, other FD&C Blue colors, titanium dioxide, iron oxide, and/or combinations thereof. Other colorants include commercially available pigments such as FD&C Green #3.

As used herein, a “lubricant” is an excipient that is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

### II. PHARMACEUTICAL COMPOSITIONS AND METHODS OF PREPARING SAME

A. Active Pharmaceutical Ingredient (API).

Compound (1), represented by the following structural formula:

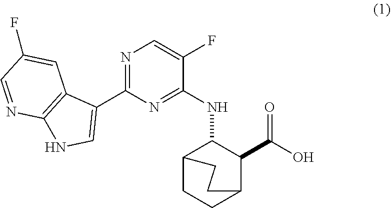

and pharmaceutically acceptable salts (and hydrates) thereof can inhibit the replication of influenza viruses as also described in WO 2010/148197. The present invention employs a crystalline HCl salt of Compound (1) hemihydrate (e.g., Compound (1) HCl hemihydrate crystalline salt Form A) in the formulations and pharmaceutical compositions described herein.

Compound (1) HCl hemihydrate crystalline salt Form A is a polymorphic form of the HCl salt of Compound (1) hemihydrate, wherein the ratio of Compound (1), HCl, and H₂O is 2:2:1, respectively. Polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds. Generally, different polymorphs can be characterized by analytical methods such as X-ray powder diffraction (XRPD) pattern, thermogravimetric analysis (TGA), and differential scanning calorimetry (DSC), or by its melting point, or other techniques known in the art. As used herein, the term “polymorphic form” includes solvates and neat polymorphic forms that lack any solvates.

As used herein, “Compound (1)” means the free base form of Compound (1). Accordingly, “HCl salt of Compound (1)” means an HCl salt of the free base of Compound (1). It is noted that HCl salts of Compound (1) can be solvated or non-solvated unless otherwise specified. The term “HCl salt of Compound (1).xH₂O” includes hydrates of HCl salt of Compound (1) when x is not zero (e.g., x is 0.5 (or ½), 1, 2, or 3), and anhydrous HCl salts of Compound (1) when x is zero. It is also noted that HCl salts of Compound (1).xH₂O can be crystalline or amorphous unless otherwise specified.

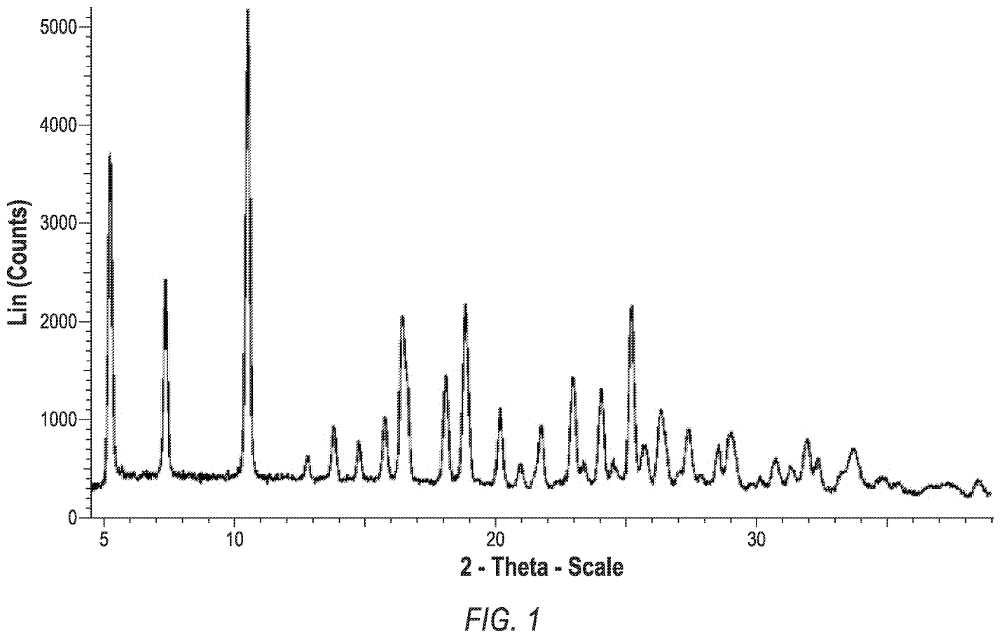

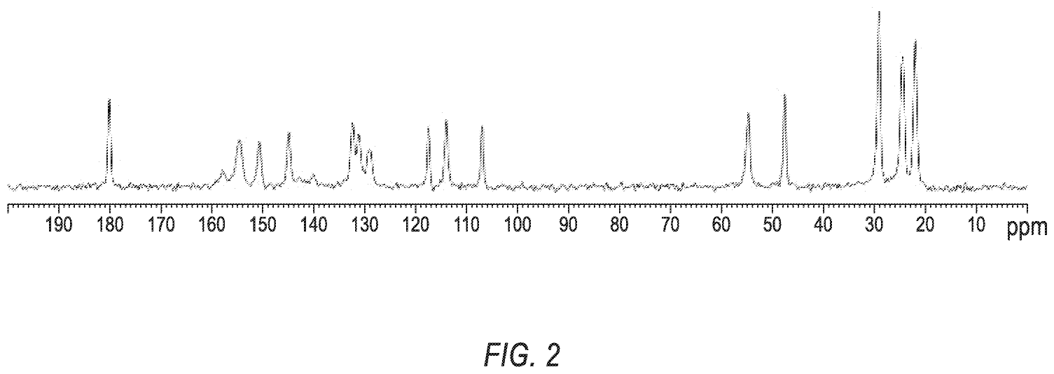

In one embodiment, the present invention employs a crystalline HCl salt of Compound (1).½H₂O (e.g., Compound (1) HCl hemihydrate crystalline salt Form A). This form is a polymorphic form of an HCl salt of Compound (1) that includes water as a solvate in a half equivalent per Compound (1). In one embodiment, Compound (1) HCl hemihydrate crystalline salt Form A is characterized as having an XRPD pattern with characteristic peaks measured 2-theta (degrees) at 10.5±0.2, 5.2±0.2, 7.4±0.2, and 12.8±0.2. In another embodiment, Compound (1) HCl hemihydrate crystalline salt Form A is characterized as having an XRPD pattern with characteristic peaks expressed in 2-theta (degrees) at the following positions listed in Table 3 of the Examples. In yet another embodiment, Compound (1) HCl hemihydrate crystalline salt Form A is characterized as having an XRPD pattern substantially the same as that shown in FIG. 1. The XRPD patterns are obtained at room temperature using Cu K alpha radiation. In yet another embodiment, Compound (1) HCl hemihydrate crystalline salt Form A is characterized as having peaks at 29.2, 107.0, 114.0, and 150.7 (0.3 ppm) in a ¹³C SSNMR spectrum. In yet another embodiment, Compound (1) HCl hemihydrate crystalline salt Form A is characterized as having ¹³C SSNMR peaks listed in Table 4 of the Examples. In yet another embodiment, Compound (1) HCl hemihydrate crystalline salt Form A is characterized as having a solid state ¹³C SSNMR spectrum substantially the same as that shown in FIG. 2.

Compound (1) HCl hemihydrate crystalline salt Form A described above can be in isolated, pure form, or in a mixture as a solid composition when admixed with other materials, for example the other solid forms (e.g., amorphous form, Form A of Compound (1), or the like) of Compound (1) or any other materials.

In some embodiments, Compound (1) HCl hemihydrate crystalline salt Form A in an isolated solid form is employed in the invention. In other embodiments, Compound (1) HCl hemihydrate crystalline salt Form A in pure form is employed in the invention. The pure form means that, for example, Compound (1) HCl hemihydrate crystalline salt Form A is over 95% (w/w), for example, over 98% (w/w), over 99% (w/w %), over 99.5% (w/w), over 99.9% (w/w), from 95% (w/w) to about 99.9% (w/w), from about 96% (w/w) to about 99.9%, or 97% (w/w) to about 99.9% (w/w) pure. In some embodiments, Compound (1) HCl hemihydrate crystalline salt Form A is in the form of a composition or a mixture of the polymorphic form with one or more other crystalline, solvate, amorphous, or other polymorphic forms or their combinations thereof.

In some embodiments, the pharmaceutical composition may comprise trace amounts up to 100% Compound (1) HCl hemihydrate crystalline salt Form A, or any amount in between, for example, 0.1%-0.5%, 0.1%-1%, 0.1%-2%, 0.1%-5%, 0.1%-10%, 0.1%-20%, 0.1%-30%, 0.1%-40%, or 0.1%-50% by weight based on the total amount of Compound (1) in the pharmaceutical composition. In yet another specific embodiment, the composition may comprise at least 50%, 60%, 70%, 80%, 90%, 95%, 97%, 98%, 99%, 99.5% or 99.9% by weight of Compound (1) HCl hemihydrate crystalline salt Form A based on the total amount of Compound (1) HCl hemihydrate in the pharmaceutical composition.

Compound (1) HCl hemihydrate crystalline salt Form A can be prepared by employing mixing (e.g., stirring) hydrogen chloride (HCl) with Compound (1). Compound (1) can be solvated, non-solvated, amorphous, or crystalline. A solution, slurry, or suspension of Compound (1) can be mixed with HCl in a solvent system that includes water and one or more organic solvents, wherein the solvent system has a water activity of equal to, or greater than, 0.05 and equal to, or less than, 0.85, i.e., 0.05-0.85. The term “water activity” (a_{w}) is used herein as known in the art and means a measure of the energy status of water in a solvent system. It is defined as the vapor pressure of a liquid divided by that of pure water at the same temperature. Specifically, water activity is defined as

$${a}_{w} = \frac{p}{{p}_{o}} ,$$

where p is the vapor pressure of water in the substance, and pₒ is the vapor pressure of pure water at the same temperature, or as a_{w}=l_{w}×x_{w}, where l_{w} is the activity coefficient of water and xo is the mole fraction of water in the aqueous fraction. For example, pure water has a water activity value of 1.0. Water activity values can typically be obtained by either a capacitance hygrometer or a dew point hygrometer. Various types of water activity measuring instruments are also commercially available. Alternatively, water activity values of mixtures of two or more solvents can be calculated based on the amounts of the solvents and the known water activity values of the solvents.

The solvent systems suitable for the preparation of Form A of the HCl salt of Compound (1).½H₂O can be comprised of a large variety of combinations of water and organic solvents where the water activity of the solvent systems is equal to, or greater than, 0.05 and equal to, or less than, 0.85 (e.g., 0.05-0.85). In one embodiment, the value of the water activity is 0.4-0.6. Suitable organic solvents include Class II or Class III organic solvents listed in the International Conference on Harmonization Guidelines. Examples of suitable Class II organic solvents include chlorobenzene, cyclohexane, 1,2-dichloroethene, dichloromethane (DCM), 1,2-dimethoxyethane, N,N-dimethylacetamide, N,N-dimethylformamide, 1,4-dioxane, 2-ethoxyethanol, formamide, hexane, 2-methoxyethanol, methylbutyl ketone, methylcyclohexane, N-methylpyrrolidone, nitromethane, pyridine, sulfolane, tetrahydrofuran (THF), tetralin, toluene, 1,1,2-trichloroethene and xylene. Examples of suitable Class III organic solvents include: acetic acid, acetone, anisole, 1-butanol, 2-butanol, butyl acetate, tert-butylmethyl ether, cumene, heptane, isobutyl acetate, isopropyl acetate, methyl acetate, 3-methyl-1-butanol, methylethyl ketone, methylisobutyl ketone, 2-methyl-1-propanol, ethyl acetate, ethyl ether, ethyl formate, pentane, 1-pentanol, 1-propanol, 2-propanol and propyl acetate. In one embodiment, the organic solvents of the solvent system are selected from the group consisting of chlorobenzene, cyclohexane, 1,2-dichloroethane, dichloromethane, 1,2-dimethoxyethane, hexane, 2-methoxyethanol, methylbutyl ketone, methylcyclohexane, nitromethane, tetralin, xylene, toluene, 1,1,2-trichloroethane, acetone, anisole, 1-butanol, 2-butanol, butyl acetate, t-butylmethylether, cumene, ethanol, ethyl acetate, ethyl ether, ethyl formate, heptane, isobutyl acetate, isopropyl acetate, methyl acetate, 3-methyl-1-butanol, methylethyl ketone, 2-methy-1-propanol, pentane, 1-propanol, 1-pentanol, 2-propanol, propyl acetate, tetrahydrofuran, and methyl tetrahydrofuran. In another embodiment, the organic solvents of the solvent system are selected from the group consisting of 2-ethoxyethanol, ethyleneglycol, methanol, 2-methoxyethanol, 1-butanol, 2-butanol, 3-methyl-1-butanol, 2-methyl-1-propanol, ethanol, 1-pentanol, 1-propanol, 2-propanol, methylbutyl ketone, acetone, methylethyl ketone, methylisobutyl ketone, butyl acetate, isobutyl acetate, isopropyl acetate, methyl acetate, ethyl acetate, propyl acetate, pyridine, toluene, and xylene. In yet another embodiment, the organic solvents are selected from the group consisting of acetone, n-propanol, isopropanol, iso-butylacetate, and acetic acid. In yet another embodiment, the organic solvents are selected from the group consisting of acetone and isopropanol. In yet another embodiment, the solvent system includes water an acetone. In yet another embodiment, the solvent system includes water an isopropanol.

The preparation of Compound (1) HCl hemihydrate salt Form A can be performed at any suitable temperature. Typically, it is performed at a temperature of 5° C.-75° C. In one embodiment, it is performed at a temperature of 15° C.-75° C. In another embodiment, it is performed at a temperature of 15° C.-60° C. In yet another embodiment, it is performed at a temperature of 15° C.-35° C. In yet another embodiment, the preparation is performed at 5° C.-75° C. in a solvent system having a water activity value of 0.4-0.6. In yet another embodiment, the preparation is performed at a temperature of 15° C.-75° C. in a solvent system having a water activity value of 0.4-0.6. In yet another embodiment, the preparation is performed at a temperature of 15° C.-60° C. in a solvent system having a water activity value of 0.4-0.6. In yet another embodiment, the preparation is performed at 15° C.-35° C. in a solvent system having a water activity value of 0.4-0.6.

The hydrogen chloride can be introduced as a solution or gas. One example of suitable hydrogen chloride source is a solution of hydrogen chloride of 30-40 weight percent (e.g., 34 wt %-38 wt %), by weight of the solution, in water.

Pharmaceutical compositions of the present invention comprise 5 wt % to 95 wt % of crystalline HCl salt of Compound (1).xH₂O (x=0, 0.5, 1, 2, or 3) (e.g., Compound (1) HCl hemihydrate salt (e.g., Compound (1) HCl hemihydrate salt Form A)) by the weight of the pharmaceutical composition, and 5 wt % to 95 wt % of one or more excipients selected from a filler or diluent, a disintegrant, a binder, a wetting agent, a lubricant, a glidant, and a coating, by the weight of the pharmaceutical composition. In some embodiments, the pharmaceutical composition comprises 40 wt % to 60 wt % (e.g., 45 wt % to 55 wt % or 47.5 wt % to 52.5 wt %) of crystalline HCl salt of Compound (1)₂H₂O (e.g., Compound (1) HCl hemihydrate crystalline salt Form A).

Unless stated otherwise, the terms “wt %” and “weight percent” are used interchangeably to refer to the concentration of an ingredient (e.g., excipient or active pharmaceutical ingredient) by weight of the pharmaceutical composition.

The wetting agents, binders, glidants, disintegrants, lubricants, and diluents suitable for the invention are compatible with the ingredients of the pharmaceutical compositions of the invention—for example, they do not substantially reduce the chemical stability of crystalline Compound (1) HCl hemihydrate.

B. Diluents/Fillers.

Diluents (or fillers) useful in the present invention include microcrystalline celluloses (e.g., Avicel® PH 101, Ceolus UF, Ceolus KG, or Ceolus PH), silicified microcrystalline celluloses, lactoses, sorbitols, celluloses, calcium phosphates, starches (e.g., partially or fully pregelatinized maize starch), sugars (e.g., mannitol, sucrose, or the like), or any combination thereof. Examples of microcrystalline celluloses include commercially available Avicel® series, such as microcrystalline celluloses having a particle size of 200 mesh over 70% and a particle size of 65 mesh less than 10% (e.g., Avicel® PH 101). Microcrystalline celluloses also include commercially available Ceolus in UF, KG, or PH grade. Other examples of diluents include silicified microcrystalline celluloses, such as commercially available Prosolv® series (e.g., Prosolv® SMCC 50 and SMCC HD90). And, lactoses suitable for the invention includes lactose monohydrate. Amounts of the diluents relative to the total weight of the pharmaceutical composition may be 5 wt % to 95 wt %, 20 wt % to 80 wt %, 25 wt % to 50 wt %, 30 wt % to 48 wt %, 35 wt % to 52 wt %, or 50 wt % to 52 wt %. For example, the diluent in the pharmaceutical composition may comprise microcrystalline cellulose, silicified microcrystalline cellulose, and partially or fully pregelatinized maize starch having a combined (or total) concentration of 5 wt % to 95 wt %, 20 wt % to 80 wt %, 25 wt % to 50 wt %, 30 wt % to 48 wt %, 35 wt % to 52 wt %, 50 wt % to 52 wt %, 30 wt % to 35 wt % or 32.5 wt % to 35 wt % by weight of the pharmaceutical composition.

C. Disintegrants.

Disintegrants enhance the dispersal of pharmaceutical compositions. Non-limiting examples of disintegrants that are useful in the present invention include croscarmelloses (e.g., croscarmellose sodium), crospovidone, metal starch glycolates (e.g., sodium starch glycolate), and any combination thereof. Other examples of disintegrants include croscarmellose sodium (e.g., Ac-Di-Sol®) and sodium starch glycolate. Pharmaceutical compositions of the present invention may comprise one or more disintegrants giving a combined (or total) concentration of 1 wt % to 10 wt %, 6 wt % to 8 wt %, 6.5 wt % to 7.5 wt %, 6.75 wt % to 7.25 wt %, 3 wt % to 7 wt %, 1 wt % to 5 wt %, or 1.2 wt % to 2.2 wt % of the pharmaceutical composition. In some embodiments, the pharmaceutical composition comprises 6 wt % to 8 wt % (e.g., 6.5 wt % to 7.5 wt %) of disintegrant (e.g., crospovidone) by weight of the pharmaceutical composition.

D. Binders.

Binders may include agents used while making granules of the active pharmaceutical ingredient by mixing the binder(s) with diluent and the active pharmaceutical ingredient. Non-limiting examples of binders useful in the present invention include polyvinyl pyrrolidones, sugar, modified celluloses (e.g., hydroxypropyl methylcelluloses (HPMC), hydroxy propyl celluloses (HPC), and hydroxy ethyl celluloses (HEC)), and any combination thereof. Other examples of the binders include polyvinyl pyrrolidones (PVP). An example of HPC includes a low viscosity polymer, HPC-SL. PVP may be characterized by its “K-value”, which is a useful measure of the polymeric composition's viscosity. PVP can be commercially purchased (e.g., Tokyo Chemical Industry Co., Ltd.) under the trade name of Povidone® K12, Povidone® K17, Povidone® K25, Povidone® K30, Povidone® K60, and Povidone® K90. Specific examples of PVP include soluble spray dried PVP. Another example includes PVP having an average molecular weight of 3,000 to 4,000, such as Povidone® K12 having an average molecular weight of 4,000. PVP can be used in either wet or dry state. Pharmaceutical compositions of the present invention may comprise one or more binders giving a combined (or total) concentration of 0.1 wt % to 5 wt %, or 0.5 wt % to 2 wt % of the pharmaceutical composition. In some embodiments, the pharmaceutical composition comprises 0.5 wt % to 2 wt % (e.g., 1.5 wt % to 2.0 wt % or 1.75 wt % to 2.25 wt %) of binder (e.g., hydroxypropyl methylcellulose) by weight of the pharmaceutical composition.

E. Lubricants.

Lubricants function to improve the compression and ejection of pharmaceutical compositions from, e.g., a die press. Non-limiting examples of lubricants useful in the present invention include magnesium stearate, stearic acid (stearin), hydrogenated oil, sodium stearyl fumarate, and any combination thereof. In one example, the lubricant includes sodium stearyl fumarate. In another example, the lubricant includes magnesium stearate. Pharmaceutical compositions of the present invention may comprise one or more lubricants giving a combined (or total) concentration of 1 wt % to 10 wt %, 0.5 wt % to 6 wt %, 0.5 wt % to 3 wt %, 1 wt % to 3 wt %, 4 wt % to 6 wt %, 4.5 wt % to 5.5 wt %, or 4.75 wt % to 5.25 wt % by weight of the pharmaceutical composition. In some embodiments, the pharmaceutical composition comprises 4.5 wt % to 5.5 wt % of lubricant (e.g., sodium stearyl fumarate).

F. Wetting Agents/Surfactants.

One or more wetting agents can be employed in the pharmaceutical compositions of the invention. Wetting agents suitable for the present invention generally enhance the solubility of pharmaceutical compositions. Wetting agents include surfactants, such as non-ionic surfactants and anionic surfactants. Non-limiting examples of surfactants useful in the invention include sodium lauryl sulfate (SLS), polyoxyethylene sorbitan fatty acids (e.g., polysorbate 20 (e.g., TWEEN 20™)), sorbitan fatty acid esters (e.g., Spans®), sodium dodecylbenzene sulfonate (SDBS), dioctyl sodium sulfosuccinate (Docusate), dioxycholic acid sodium salt (DOSS), sorbitan monostearate, sorbitan tristearate, sodium N-lauroylsarcosine, sodium oleate, sodium myristate, sodium stearate, sodium palmitate, gelucire 44/14, ethylenediamine tetraacetic acid (EDTA), vitamin E d-alpha tocopheryl polyethylene glycol 1000 succinate (TPGS), lecithin, MW 677-692, glutanic acid monosodium monohydrate, labrasol, PEG 8 caprylic/capric glycerides, transcutol, diethylene glycol monoethyl ether, solutol HS-15, polyethylene glycol/hydroxystearate, taurocholic acid, copolymers of polyoxypropylene and polyoxyethylene (e.g., poloxamers also known and commercially available under Pluronics®, such as, Pluronic® L61, Pluronic® F68, Pluronic® F108, and Pluronic® F127), saturated polyglycolized glycerides (Gelucirs®), and any combination thereof. Other examples include sodium lauryl sulfate, which is an anionic surfactant; and copolymers of polyoxypropylene and polyoxyethylene which are non-ionic surfactants. Examples of the copolymers of polyoxypropylene and polyoxyethylene include poloxamers, such as poloxamer with a polyoxypropylene molecular mass of 1,800 g/mol and an 80% polyoxyethylene content (e.g., poloxamer 188). Pharmaceutical compositions of the present invention may comprise one or more wetting agents giving a combined (or total) concentration of 0.25 wt % to 10 wt %, 1 wt % to 5 wt %, or 0.25 wt % to 0.75 wt % by weight of the pharmaceutical composition. In some embodiments, the pharmaceutical composition comprises 0.25 wt % to 0.75 wt % (e.g., 0.35 wt % to 0.65 wt %) of a wetting agent (e.g., polysorbate 20).

G. Glidants.

Glidants enhance the flow properties of formulations during processing into final drug product form. Non-limiting examples of glidants useful in the present invention include silicon dioxide (e.g., colloidal anhydrous silica) and/or talc. Pharmaceutical compositions of the present invention may comprise one or more wetting agents giving a combined (or total) concentration of 1 wt % to 10 wt % by the weight of the pharmaceutical composition. In some embodiments, the pharmaceutical composition comprises colloidal anhydrous silica in an amount of 0.10 wt % to 2 wt % (e.g., 0.75 wt % to 1.25 wt %) by weight of the pharmaceutical composition. In other embodiments, the pharmaceutical composition comprises talc in an amount of 0.10 wt % to 2 wt % (e.g., 0.75 wt % to 1.25 wt %) by weight of the pharmaceutical composition.

H. Pharmaceutical Compositions.

In one aspect, the present invention provides a pharmaceutical composition comprising (a) 40 wt % to 60 wt % (e.g., 45 wt % to 55 wt % or 47.5 wt % to 52.5 wt %) of crystalline HCl salt of Compound (1)₂H₂O (e.g., Compound (1) HCl hemihydrate salt Form A), (b) 20 wt % to 80 wt % (e.g., 30 wt % to 48 wt %, 35 wt % to 52 wt %, 50 wt % to 52 wt %, 30 wt % to 35 wt % or 32.5 wt % to 35 wt %) of diluent (e.g., microcrystalline cellulose, silicified microcrystalline cellulose, partially or fully gelatinized maize starch, or any combination thereof), (c) 6 wt % to 8 wt % (e.g., 6.5 wt % to 7.5 wt %) of disintegrant (e.g., crospovidone), (d) 0.5 wt % to 2 wt % (e.g., 1.5 wt % to 2.0 wt % or 1.75 wt % to 2.25 wt %) of binder (e.g., hydroxypropyl methylcellulose), (e) 1 wt % to 10 wt %, 0.5 wt % to 6 wt %, 0.5 wt % to 3 wt %, 1 wt % to 3 wt %, 4 wt % to 6 wt %, 4.5 wt % to 5.5 wt %, or 4.75 wt % to 5.25 wt % of lubricant (e.g., sodium stearyl fumarate), (f) 0.25 wt % to 0.75 wt % (e.g., 0.35 wt % to 0.65 wt %) of a wetting agent (e.g., polysorbate 20), and (g) 0.10 wt % to 2 wt % (e.g., 0.75 wt % to 1.25 wt %) of glidant (e.g., colloidal anhydrous silica).

In one aspect, the present invention provides a pharmaceutical composition comprising (a) a plurality of granules forming an intragranular phase of the composition comprising (i) a crystalline HCl salt of Compound (1).xH₂O (x=0, 0.5 (or ½), 1, 2, or 3) (e.g., a crystalline HCl salt of Compound (1).½H₂O (e.g., Compound (1) crystalline HCl hemihydrate salt Form A)) wherein Compound (1) is represented by the following structural formula:

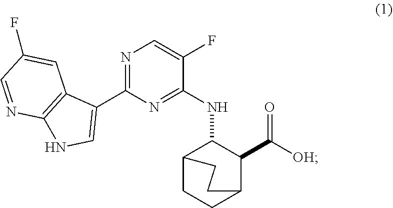

and (ii) one or more excipients selected from a disintegrant, a binder, and a wetting agent; and (b) an extragranular phase of the composition comprising a diluent, a disintegrant, a glidant, and a lubricant, wherein the HCl salt of Compound (1).xH₂O has a concentration of 5 wt % to 95 wt % (e.g., 45 wt % to 55 wt % or 47.5 wt % to 52.5 wt %) by weight of the composition, and the combined concentration of the disintegrant, binder, wetting agent, diluent, glidant, and a lubricant is 5 wt % to 95 wt % (e.g., 45 wt % to 55 wt % or 47.5 wt % to 52.5 wt %) by weight of the composition.

In some embodiments, the crystalline HCl salt of Compound (1).xH₂O is crystalline HCl salt of Compound (1).½H₂O (i.e., the crystalline HCl salt of Compound (1) hemihydrate). For instance, the crystalline HCl salt of Compound (1).½H₂O is Compound (1) crystalline HCl hemihydrate salt Form A.

In some embodiments, the composition comprises 10 wt % to 80 wt % of a diluent by weight of the pharmaceutical composition. In some examples, the diluent comprises microcrystalline cellulose, starch, silica, or any combination thereof. For example, the diluent comprises 4 wt % to 6 wt % (e.g., 4.5 wt % to 6.5 wt %, 4.75 wt % to 6.25 wt %, 4.85 wt % to 5.25 wt %, or about 5 wt %, about 5.25 wt %, or about 4.3 wt %) by weight of the pharmaceutical composition of microcrystalline cellulose. In other examples, the diluent comprises 15 wt % to 30 wt % (e.g., 17.5 wt % to 25 wt %, 20 wt % to 25 wt %, 22 wt % to 24 wt %, about 23.18 wt %, about 26.5 wt %, or about 27 wt %) by weight of the pharmaceutical composition of silicified microcrystalline cellulose. In other examples, the diluent comprises 4 wt % to 6 wt % (e.g., 4.5 wt % to 6.5 wt %, 4.75 wt % to 6.25 wt %, 4.85 wt % to 5.25 wt %, or about 5.25 wt %, about 5 wt %, or about 6.15 wt %) by weight of the pharmaceutical composition of partially or fully gelatinized starch. And, in some examples, the diluent comprises 4 wt % to 6 wt % (e.g., 4.5 wt % to 6.5 wt %, 4.75 wt % to 6.25 wt %, 4.85 wt % to 5.25 wt %, or about 5 wt %, about 5.25 wt %, or about 4.3 wt %) by weight of the pharmaceutical composition of microcrystalline cellulose, 15 wt % to 30 wt % (e.g., 17.5 wt % to 25 wt %, 20 wt % to 25 wt %, 22 wt % to 24 wt %, about 23.18 wt %, about 26.5 wt %, or about 27 wt %) by weight of the pharmaceutical composition of silicified microcrystalline cellulose, and 4 wt % to 6 wt % (e.g., 4.5 wt % to 6.5 wt %, 4.75 wt % to 6.25 wt %, 4.85 wt % to 5.25 wt %, or about 5.25 wt %, about 5 wt %, or about 6.15 wt %) by weight of the pharmaceutical composition of partially or fully pregelatinized maize starch giving a combined concentration of 23 wt % to 42 wt % (e.g., 30 wt % to 35 wt % or 32.5 wt % to 35 wt %) by weight of the pharmaceutical composition of diluent in the pharmaceutical composition.

In other embodiments, the pharmaceutical composition comprises 20 wt % to 27.5 wt % (e.g., 22 wt % to 24 wt %, or from 22.5 wt % to 23.5 wt %) of silicified microcrystalline cellulose and/or 2.5 wt % to 7.5 wt % (e.g., 4 wt % to 6 wt % or 4.5 wt % to 5.5 wt %) of microcrystalline cellulose (i.e., non-silicified microcrystalline cellulose) by weight of the pharmaceutical composition.

In some embodiments, the composition comprises 1 wt % to 10 wt % of a disintegrant by the weight of the pharmaceutical composition. In some examples, the disintegrant comprises croscarmellose sodium, crospovidone, or any combination thereof. For instance, the pharmaceutical composition comprises crospovidone in an amount of 1 wt % to 2 wt % (e.g., 1.25 wt % to 1.75 wt %) or 4 wt % to 10 wt % (e.g., 6 wt % to 8 wt %, 6.5 wt % to 7.5 wt %, 6.25 wt % to 7.25 wt %, about 7 wt %, about 6.75 wt %, or about 7.15 wt %) by weight of the pharmaceutical composition.

In some embodiments, the composition comprises 0.1 wt % to 10 wt % of a binder by weight of the pharmaceutical composition. In some embodiments, the composition comprises 0.1 wt % to 5 wt % of a binder by the weight of the pharmaceutical composition. In some examples, the binder comprises hydroxypropyl methylcellulose. For instance, the pharmaceutical composition comprises hydroxypropyl methylcellulose in an amount of 1.5 wt % to 2.5 wt % (e.g., 1.75 wt % to 2.25 wt %, 1.85 wt % to 2.0 wt %, about 1.8 wt %, about 1.9 wt %, or about 2.2 wt %) by weight of the pharmaceutical composition.

In some embodiments, the composition comprises 0.5 wt % to 10 wt % of a lubricant by weight of the pharmaceutical composition. In some embodiments, the composition comprises 0.5 wt % to 6 wt % of a lubricant by the weight of the pharmaceutical composition. In some examples, the lubricant comprises sodium stearyl fumarate, magnesium stearate, or any combination thereof. For instance, the pharmaceutical composition comprises sodium stearyl fumarate in an amount of 4 wt % to 6 wt % (e.g., 4.5 wt % to 5.5 wt %, 4.6 wt % to 5.2 wt %, about 5 wt %, about 4.8 wt %, or about 5.7 wt %) by weight of the pharmaceutical composition.

In some embodiments, the composition comprises 0.1 wt % to 1.0 wt % of a wetting agent by the weight of the pharmaceutical composition. In some examples, the wetting agent comprises polysorbate 20 (e.g., Tween 20). For instance, the pharmaceutical composition comprises polysorbate 20 in an amount of 0.10 wt % to 1 wt % (e.g., 0.25 wt % to 0.75 wt %, 0.35 wt % to 0.65 wt %, about 0.45 wt %, about 0.55 wt % or about 0.65 wt %) by weight of the pharmaceutical composition.

In some embodiments, the composition comprises 0.1 wt % to 10 wt % of a glidant by the weight of the pharmaceutical composition. In some embodiments, the composition comprises 1 wt % to 10 wt % of a glidant by the weight of the pharmaceutical composition. In some examples, the glidant comprises silicon dioxide (e.g., colloidal anhydrous silica). For instance, the pharmaceutical composition comprises colloidal anhydrous silica in an amount of 0.10 wt % to 2 wt % (e.g., 0.75 wt % to 1.75 wt %, 0.75 wt % to 1.25 wt %, about 1 wt %, about 0.9 wt %, or about 1.4 wt %) by weight of the pharmaceutical composition.

In some embodiments, the pharmaceutical composition comprises (a) 20 wt % to 80 wt % (e.g., 30 wt % to 70 wt %, 40 wt % to 70 wt %, or 49 wt % to 54 wt %) of Compound (1) crystalline HCl hemihydrate crystalline salt Form A by weight of the pharmaceutical composition; (b) 1 wt % to 10 wt % (e.g., 1 wt % to 2 wt %, 1.25 wt % to 1.75 wt %, 1.4 wt % to 1.6 wt %, 6 wt % to 8 wt %, or 6.5 wt % to 7.5 wt %) of the disintegrant (e.g., crospovidone) by weight of the pharmaceutical composition; (c) 1 wt % to 10 wt % (e.g., 1 wt % to 5 wt %, 1 wt % to 3 wt %, 1.54 wt % to 2.00 wt %, or 1.85 wt % to 1.95 wt %) of the binder (e.g., hydroxypropyl methylcellulose) by weight of the pharmaceutical composition; (d) 0.1 wt % to 1.0 wt % (e.g., 0.1 wt % to 0.6 wt %, 0.3 wt % to 0.6 wt %, 0.15 wt % to 0.55 wt %, 0.20 wt % to 0.3 wt %, or 0.45 wt % to 0.55 wt %) of the wetting agent (e.g., polysorbate 20) by the weight of the pharmaceutical composition; (e) 0.1 wt % to 5.0 wt % (e.g., 0.5 wt % to 2.0 wt %, 0.5 wt % to 1.5 wt %, or 0.95 wt % to 1.05 wt %) of the glidant (e.g., colloidal anhydrous silica) by weight of the pharmaceutical composition; (f) 1 wt % to 10 wt % (e.g., 1.5 wt % to 6 wt %, 1.5 wt % to 5.5 wt %, 2.85 wt % to 3.15 wt %, 1.85 wt % to 2.15 wt %, or 4.85 wt % to 5.15 wt %) of the lubricant (e.g., sodium stearyl fumarate) by weight of the pharmaceutical composition; and (g) 20 wt % to 80 wt % (e.g., 20 wt % to 45 wt %, 25 wt % to 40 wt %, 34 wt % to 38 wt %, or 25.0 wt % to 27.0 wt %) of the diluent (e.g., microcrystalline cellulose, silicified microcrystalline cellulose, starch (e.g., partially or fully pregelatinized maize starch), or any combination thereof) by weight of the pharmaceutical composition.

In some embodiments, the pharmaceutical composition comprises (a) an intragranular phase comprising (or consisting essentially of) (i) 49 wt % to 54 wt % (e.g., 49 wt % to 52 wt %, 49.5 wt % to 51.5 wt %, about 49.5 wt %, about 50.25 wt %, or about 51.5 wt %) of crystalline HCl sale of Compound (1) hemihydrate (e.g., Compound (1) HCl hemihydrate crystalline salt Form A) by weight of the pharmaceutical composition; (ii) 1.45 wt % to 1.62 wt % of the disintegrant (e.g., crospovidone) by weight of the pharmaceutical composition; (iii) 1.54 wt % to 1.70 wt % or 1.85 wt % to 1.95 wt % of the binder (e.g., hydroxypropyl methylcellulose) by weight of the pharmaceutical composition; and (d) 0.21 wt % to 0.25 wt % or 0.45 wt % to 0.55 wt % of the wetting agent (e.g., polysorbate 20) by weight of the pharmaceutical composition.

Some embodiments further comprise (b) an extragranular phase comprising (or consisting essentially of) (i) 5.0 wt % to 6.0 wt % of disintegrant by weight of the pharmaceutical composition; (b) 0.95 wt % to 1.05 wt % of the glidant by weight of the pharmaceutical composition; (c) 2.85 wt % to 3.15 wt % of the lubricant by weight of the pharmaceutical composition; and (d) 34 wt % to 38 wt % of the diluent by weight of the pharmaceutical composition.

In some alternative embodiments, the extragranular phase of the composition comprises (i) 27.0 wt % to 35.0 wt % (e.g., 30 wt % to 35 wt %, 32.5 wt % to 35 wt %, about 33 wt %, about 35 wt %, or about 34 wt %) of the diluent (e.g., the diluent comprises microcrystalline cellulose, silicified microcrystalline cellulose, and partially or fully pregelatinized maize starch) by weight of the pharmaceutical composition; (ii) 0.95 wt % to 1.05 wt % (e.g., about 1 wt %) of the glidant (e.g., colloidal anhydrous silica) by weight of the pharmaceutical composition; (iii) 3 wt % to 6 wt % (e.g., 4.75 wt % to 5.25 wt %, about 4.9 wt %, about 5 wt % or about 5.2 wt %) of the lubricant (e.g., sodium stearyl fumarate) by weight of the pharmaceutical composition; and (iv) 2.5 wt % to 7.5 wt % (e.g., 3.0 wt % to 7.25 wt %, 4.5 wt % to 6.5 wt %, 5 wt % to 6 wt %, 5.25 wt % to 5.75 wt %, about 5 wt %, about 6 wt %, or about 5.5 wt %) of disintegrant (e.g., crospovidone) by weight of the pharmaceutical composition.

In some embodiments, the crystalline Compound (1) HCl hemihydrate salt (e.g., Compound (1) HCl hemihydrate crystalline salt Form A)) is in a micronized state in the pharmaceutical composition.

In some embodiments, the crystalline Compound (1) HCl hemihydrate salt (e.g., Compound (1) HCl hemihydrate crystalline salt Form A)) is present in an amount of about 51.42 wt % by weight of the pharmaceutical composition.

In some embodiments, the disintegrant in the intragranular phase is crospovidone. In some examples, the disintegrant in the intragranular phase is present in an amount of about 1.54 wt % or about 1.50 wt % by weight of the pharmaceutical composition.

In some embodiments, the binder is hydroxypropyl methylcellulose. In some examples, the binder is hydroxypropyl methylcellulose having a viscosity of about 15 mPa·s. And, in some examples, the binder is present in the intragranular phase in an amount of about 1.54 wt % or about 1.90 wt % by weight of the pharmaceutical composition.

In some embodiments, the wetting agent is a polysorbate. In some examples, the wetting agent is polysorbate 20. In some examples, the wetting agent is present in the intragranular phase in an amount of about 0.23 wt % or about 0.50 wt % by weight of the pharmaceutical composition.

In some embodiments, the glidant is colloidal anhydrous silica. In some examples, the glidant is present in the extragranular phase in an amount of about 1.00 wt %, about 0.85 wt %, or about 1.2 wt % by weight of the pharmaceutical composition.

In some embodiments, the disintegrant in the extragranular phase is crospovidone. In some examples, the disintegrant in the extragranular phase is present in an amount of about 5.46 wt % or about 5.5 wt % by weight of the pharmaceutical composition.

In some embodiments, the lubricant is sodium stearyl fumarate. In some examples, the lubricant is present in the extragranular phase in an amount of about 3.00 wt % or about 5 wt % by weight of the pharmaceutical composition.

In some embodiments, the diluent comprises silicified microcrystalline cellulose, microcrystalline cellulose, starch, or any combination thereof. In some examples, the starch is partially or fully pregelatinized maize starch (e.g., Starch 1500@). In some examples, the diluent is present in an amount of about 35.74 wt % or about 33.18 wt % by weight of the pharmaceutical composition. In some examples, the diluent comprises silicified microcrystalline cellulose. For instance, the silicified microcrystalline cellulose is present in an amount of about 25.74 wt % or about 23.18 wt % by weight of the pharmaceutical composition. In other examples, the diluent comprises silicified microcrystalline cellulose and starch (e.g., partially or fully pregelatinized maize starch). For instance, the silicified microcrystalline cellulose is present in an amount of about 25.74 wt % or about 23.18 wt % by weight of the pharmaceutical composition and the starch is present in an amount of about 10.00 wt % or about 5 wt % by weight of the pharmaceutical composition. And, in other examples, the diluent comprises silicified microcrystalline cellulose and microcrystalline cellulose. For instance, the silicified microcrystalline cellulose is present in an amount of about 25.74 wt % or about 23.18 wt % by weight of the pharmaceutical composition, and the microcrystalline cellulose is present in an amount of about 10.00 wt % or 5 wt % by weight of the pharmaceutical composition. In other examples, the diluent comprises silicified microcrystalline cellulose, microcrystalline cellulose, and starch. For instance, the silicified microcrystalline cellulose is present in an amount of about 25.74 wt % or about 23.18 wt % by weight of the pharmaceutical composition, the microcrystalline cellulose is present in an amount of about 5.00 wt % by weight of the pharmaceutical composition, and the starch (e.g., partially or fully pregelatinized maize starch) is present in an amount of about 5.00 wt % by weight of the pharmaceutical composition.

In some embodiments, the pharmaceutical composition comprises (a) 57.50 wt % to 64.00 wt % of crystalline Compound (1) HCl hemihydrate salt (e.g., Compound (1) HCl hemihydrate crystalline salt Form A)) by weight of the pharmaceutical composition; (b) 6.5 wt % to 7.5 wt % (e.g., 6.75 wt % to 7.25 wt % or 6.9 wt % to 7.2 wt %) of disintegrant (e.g., crospovidone) by weight of the pharmaceutical composition; (c) 1.80 wt % to 2.10 wt % of binder (e.g., hydroxypropyl methylcellulose) by weight of the pharmaceutical composition; (d) 0.25 wt % to 0.75 wt % (e.g., 0.35 wt % to 0.65 wt % or 0.45 wt % to 0.55 wt %) of wetting agent (e.g., polysorbate 20) by weight of the pharmaceutical composition; (e) 0.95 wt % to 1.05 wt % of glidant (e.g., colloidal anhydrous silica) by the weight of the pharmaceutical composition; (f) 2.50 wt % to 7.50 wt % (e.g., 4.5 wt % to 6.5 wt %, 4.75 wt % to 6.25 wt %, 4.75 wt % to 6 wt %, or 4.8 wt % to 5.2 wt %) of lubricant (e.g., sodium stearyl fumarate) by weight of the pharmaceutical composition; and (g) 20 wt % to 40 wt % (e.g., 25 wt % to 35 wt %, 30 wt % to 35 wt %, or 32 wt % to 34 wt %) of diluent by weight of the pharmaceutical composition.

In some embodiments, the pharmaceutical composition comprises (a) an intragranular phase comprising (a) 57.50 wt % to 64.00 wt % (e.g., 50 wt % to 53 wt %) of crystalline Compound (1) HCl hemihydrate salt (e.g., Compound (1) HCl hemihydrate crystalline salt Form A) by weight of the pharmaceutical composition; (b) 1.25 wt % to 1.95 wt % (e.g., 1.42 wt % to 1.58 wt %) of disintegrant by weight of the pharmaceutical composition; (c) 1.80 wt % to 2.10 wt % (e.g., 1.80 wt % to 2.00 wt %) of binder by weight of the pharmaceutical composition; and (d) 0.25 wt % to 0.75 wt % (e.g., 0.47 wt % to 0.53 wt %) of wetting agent by weight of the pharmaceutical composition.

In some embodiments, the crystalline Compound (1) HCl hemihydrate salt (e.g., Compound (1) HCl hemihydrate crystalline salt Form A) in the intragranular phase is in a micronized state (Microfine API) in the pharmaceutical composition. In other examples, the crystalline Compound (1) HCl hemihydrate salt (e.g., Compound (1) HCl hemihydrate crystalline salt Form A) is present in an amount of about 60.76 wt % by weight of the pharmaceutical composition.

In some embodiments, the disintegrant in the intragranular phase is crospovidone. For example, the disintegrant in the intragranular phase is present in an amount of about 1.82 wt % by weight of the pharmaceutical composition.

In some embodiments, the binder in the intragranular phase is hydroxypropyl methylcellulose. For example, the binder is hydroxypropyl methylcellulose having a viscosity of about 15 mPa·s. In other examples, the binder is present in the intragranular phase in an amount of about 1.91 wt % by weight of the pharmaceutical composition.

In some embodiments, the wetting agent is a polysorbate. For example, the wetting agent is polysorbate 20. In other examples, the wetting agent is present in an amount of about 0.27 wt % by weight of the pharmaceutical composition.

In some embodiments, the pharmaceutical composition comprises an intragranual phase (such as any intragranular phase described herein) and an extragranular phase comprising (a) 4.5 wt % to 5.7 wt % (e.g., 5.25 wt % to 5.75 wt %) of the disintegrant by weight of the pharmaceutical composition; (b) 0.95 wt % to 1.05 wt % (e.g., 0.95 wt % to 1.05 wt %) of the glidant by weight of the pharmaceutical composition; (c) 2.9 wt % to 3.1 wt % (e.g., 4.75 wt % to 5.25 wt %) of the lubricant by weight of the pharmaceutical composition; and (d) 24.5 wt % to 27.5 wt % (e.g., 31.50 wt % to 35.00 wt %) of the diluent by weight of the pharmaceutical composition.

In some embodiments, the glidant in the extragranular phase is colloidal anhydrous silica. For example, the glidant is present in the extragranular phase in an amount of about 1.00 wt % by weight of the pharmaceutical composition.

In some embodiments, the disintegrant in the extragranular phase is crospovidone. In some examples, the disintegrant in the extragranular phase is present in an amount of about 5.18 wt % by weight of the pharmaceutical composition.

In some embodiments, the lubricant in the extragranular phase is sodium stearyl fumarate. In some examples, the lubricant is present in an amount of about 3.00 wt % by weight of the pharmaceutical composition.

In some embodiments, the diluent of the extragranular phase comprises silicified microcrystalline cellulose, microcrystalline cellulose, starch, or any combination thereof. In some examples, wherein the starch is partially or fully pregelatinized maize starch. In some examples, the diluent is present in an amount of about 26.05 wt % by weight of the pharmaceutical composition. In some examples, the diluent comprises silicified microcrystalline cellulose. In some examples, the silicified microcrystalline cellulose is present in an amount of about 18.55 wt % by weight of the pharmaceutical composition. In some examples, the diluent comprises silicified microcrystalline cellulose and starch. In some examples, the silicified microcrystalline cellulose is present in an amount of about 18.55 wt % by weight of the pharmaceutical composition, and the starch is present in an amount of about 7.50 wt % by weight of the pharmaceutical composition. In some examples, the diluent comprises silicified microcrystalline cellulose and microcrystalline cellulose. In some examples, the silicified microcrystalline cellulose is present in an amount of about 18.55 wt % by weight of the pharmaceutical composition, and the microcrystalline cellulose is present in an amount of about 7.5 wt % by weight of the pharmaceutical composition. In other examples, the diluent comprises silicified microcrystalline cellulose, microcrystalline cellulose, and starch. In some examples, the silicified microcrystalline cellulose is present in an amount of about 18.55 wt % by weight of the pharmaceutical composition, the microcrystalline cellulose is present in an amount of about 3.75 wt % by weight of the pharmaceutical composition, and the starch is present in an amount of about 3.75 wt % by weight of the pharmaceutical composition.

In some embodiments, the crystalline Compound (1) HCl hemihydrate salt (e.g., Compound (1) HCl hemihydrate crystalline salt Form A) is in a micronized state in the pharmaceutical composition. In some examples, the crystalline Compound (1) HCl hemihydrate salt (e.g., Compound (1) HCl hemihydrate crystalline salt Form A) is present in an amount of about 51.42 wt % by weight of the pharmaceutical composition.

In some embodiments, the disintegrant in the intragranular phase is crospovidone. In some examples, the disintegrant in the intragranular phase is present in an amount of about 1.50 wt % by weight of the pharmaceutical composition.

In some embodiments, the binder is hydroxypropyl methylcellulose. In some examples, the binder is hydroxypropyl methylcellulose having a viscosity of about 15 mPa·s. In some examples, the binder is present in an amount of about 1.90 wt % by weight of the pharmaceutical composition.

In some embodiments, the wetting agent is a polysorbate. For example, the wetting agent is polysorbate 20. In some examples, the wetting agent is present in an amount of about 0.50 wt % by weight of the pharmaceutical composition.

In some embodiments, the glidant is colloidal anhydrous silica. In some examples, the glidant is present in an amount of about 1.00 wt % by weight of the pharmaceutical composition.

In some embodiments, the disintegrant in the extragranular phase is crospovidone. In some examples, the disintegrant in the extragranular phase is present in an amount of about 5.50 wt % by weight of the pharmaceutical composition.

In some embodiments, the lubricant is sodium stearyl fumarate. In some examples, the lubricant is present in an amount of about 5.00 wt % by weight of the pharmaceutical composition.

In some embodiments, the diluent comprises silicified microcrystalline cellulose, microcrystalline cellulose, starch, or any combination thereof. In some examples, the starch is partially or fully pregelatinized maize starch. In some examples, the diluent is present in an amount of about 33.18 wt % by weight of the pharmaceutical composition. In some examples, the diluent comprises silicified microcrystalline cellulose. In some examples, the silicified microcrystalline cellulose is present in an amount of about 23.18 wt % by weight of the pharmaceutical composition. In some examples, the diluent comprises silicified microcrystalline cellulose, microcrystalline cellulose, and starch. In some examples, the silicified microcrystalline cellulose is present in an amount of about 23.18 wt % by weight of the pharmaceutical composition, the microcrystalline cellulose is present in an amount of about 5.00 wt % by weight of the pharmaceutical composition, and the starch is present in an amount of about 5.00 wt % by weight of the pharmaceutical composition.

In another embodiment, the intragranular phase of the composition comprises: a) 50 wt % to 53 wt % of crystalline Compound (1) HCl hemihydrate salt (e.g., Compound (1) HCl hemihydrate crystalline salt Form A) by weight of the pharmaceutical composition; b) 1.42 wt % to 1.58 wt % of the disintegrant by weight of the pharmaceutical composition; c) 1.80 wt % to 2.00 wt % of the binder by weight of the pharmaceutical composition; and d) 0.47 wt % to 0.53 wt % of the wetting agent by weight of the pharmaceutical composition; and the extragranular phase of the composition comprises: a) 5.25 wt % to 5.75 wt % of the disintegrant by weight of the pharmaceutical composition; b) 0.95 wt % to 1.05 wt % of the glidant by weight of the pharmaceutical composition; c) 1.75 wt % to 2.25 wt % of the lubricant by weight of the pharmaceutical composition; and d) 34.00 wt % to 38.00 wt % of the diluent by weight of the pharmaceutical composition.

In one embodiment, the crystalline Compound (1) HCl hemihydrate salt (e.g., Compound (1) HCl hemihydrate crystalline salt Form A) is in a micronized state in the pharmaceutical composition. In another embodiment, the crystalline Compound (1) HCl hemihydrate salt (e.g., Compound (1) HCl hemihydrate crystalline salt Form A) is present in an amount of about 51.42 wt % by weight of the pharmaceutical composition.

In one embodiment, the disintegrant in the intragranular phase is crospovidone. In another embodiment, the disintegrant in the intragranular phase is present in an amount of about 1.50 wt % by weight of the pharmaceutical composition.

In one embodiment, the binder is hydroxypropyl methylcellulose. In another embodiment, the binder is hydroxypropyl methylcellulose having a viscosity of about 15 mPa·s. In another embodiment, the binder is present in an amount of about 1.90 wt % by weight of the pharmaceutical composition.

In one embodiment, the wetting agent is a polysorbate. In another embodiment, the wetting agent is polysorbate 20. In another embodiment, the wetting agent is present in an amount of about 0.50 wt % by weight of the pharmaceutical composition.

In one embodiment, the glidant is colloidal anhydrous silica. In another embodiment, the glidant is present in an amount of about 1.00 wt % by weight of the pharmaceutical composition.

In one embodiment, the disintegrant in the extragranular phase is crospovidone. In another embodiment, the disintegrant in the extragranular phase is present in an amount of about 5.50 wt % by weight of the pharmaceutical composition.

In one embodiment, the lubricant is sodium stearyl fumarate. In another embodiment, the lubricant is present in an amount of about 2.00 wt % by weight of the pharmaceutical composition.

In one embodiment, the diluent comprises silicified microcrystalline cellulose, microcrystalline cellulose, starch, or any combination thereof. In a further embodiment, the starch is partially or fully pregelatinized maize starch. In one embodiment, the diluent is present in an amount of about 36.18 wt % by weight of the pharmaceutical composition. In a further embodiment, the diluent comprises silicified microcrystalline cellulose. In still a further embodiment, the silicified microcrystalline cellulose is present in an amount of about 26.18 wt % by weight of the pharmaceutical composition. In another further embodiment, the diluent comprises silicified microcrystalline cellulose, microcrystalline cellulose, and starch. In still a further embodiment, the silicified microcrystalline cellulose is present in an amount of about 26.18 wt % by weight of the pharmaceutical composition, the microcrystalline cellulose is present in an amount of about 5.00 wt % by weight of the pharmaceutical composition, and the starch is present in an amount of about 5.00 wt % by weight of the pharmaceutical composition.

In some embodiments, the pharmaceutical composition is in the form of a tablet. In some examples, the tablet further comprises a film coating. And, in some examples, the film coating comprises a polymer, plasticizer and pigment. For instance, the film coating comprises a white pigment. In some examples, the film coating comprises a polymer, plasticizer, an anti-tacking agent, and pigment. In one embodiment, the anti-tacking agent is talc. In one embodiment, the pigment is titanium dioxide. In other instances, the film coating Opadry II White 85F18422. And, in some instances, the film coating is Opadry II Yellow 85F92450.

The present invention also provides a pharmaceutical composition comprising tablet, wherein the tablet comprises (a) a plurality of granules that form an intragranular phase of the composition, wherein the granules are produced by fluid bed granulation and comprise (i) about 51.42 wt % of crystalline Compound (1) HCl hemihydrate salt (e.g., Compound (1) HCl hemihydrate crystalline salt Form A) by weight of the pharmaceutical composition, wherein Compound (1) is represented by the following structural formula:

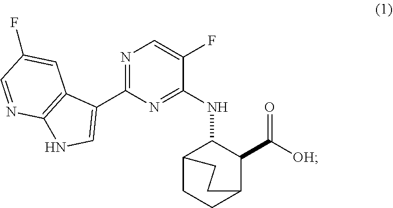

(ii) about 1.54 wt % or about 1.50 wt % of crospovidone by weight of the pharmaceutical composition; (iii) about 1.62 wt % or about 1.90 wt % of hydroxypropyl methylcellulose having a viscosity of about 15 mPa·s, by weight of the pharmaceutical composition; and (iv) about 0.23 wt % or about 0.5 wt % of polysorbate 20 by weight of the pharmaceutical composition; and (b) one or more excipients that form an extragranular phase of the composition, wherein the extragranular phase of the composition comprises (i) about 5.46 wt % or about 5.5 wt % of crospovidone by weight of the pharmaceutical composition; (ii) about 1.00 wt % of colloidal anhydrous silica by weight of the pharmaceutical composition; (iii) about 3 wt % or about 5 wt % of sodium stearyl fumarate by weight of the pharmaceutical composition; (iv) about 25.74 wt % or about 23.18 wt % of silicified microcrystalline cellulose by weight of the pharmaceutical composition; (v) about 5.00 wt % of microcrystalline cellulose by weight of the pharmaceutical composition; and (vi) about 5.00 wt % of starch by weight of the pharmaceutical composition.

In some embodiments, each tablet comprises an intragranular phase and an extragranular phase, the intragranular phase of the composition comprising (a) about 668.40 mg of crystalline Compound (1) HCl hemihydrate salt (e.g., Compound (1) HCl hemihydrate crystalline salt Form A); (b) about 20.00 mg of crospovidone; (c) about 21.00 mg of hydroxypropyl methylcellulose having a viscosity of about 15 mPa·s; and (d) about 3.00 mg of polysorbate 20; and the extragranular phase of the composition comprising (a) about 71.00 mg of crospovidone; (b) about 13.00 mg of colloidal anhydrous silica; (c) about 39.00 mg of sodium stearyl fumarate; (d) about 334.60 mg of silicified microcrystalline cellulose; (e) about 65.00 mg of microcrystalline cellulose; and (f) about 65.00 mg of starch.

In some embodiments, the pharmaceutical composition comprises a tablet, wherein the tablet comprises 300 mg to 350 mg (e.g., 330 mg to 340 mg, 332 mg to 335 mg, about 334 mg, about 335 mg, or about 336 mg) of crystalline Compound (1) HCl hemihydrate salt (e.g., Compound (1) HCl hemihydrate crystalline salt Form A) by weight of the tablet. In some of these embodiments, the tablet comprises 40 mg to 50 mg (e.g., 42.5 mg to 47.5 mg, 44 mg to 46 mg, about 44 mg, about 45 mg, or about 46 mg) of crospovidone. In some of these embodiments, the tablet comprises 10 mg to 15 mg (e.g., 12 mg to 14 mg, 12 mg to 13 mg, about 12.25 mg, about 12.35 mg, or about 12.45 mg) of hydroxypropyl methylcellulose. In some of these embodiments, the tablet comprises 1 mg to 5 mg (e.g., 2 mg to 4 mg, 2.5 mg to 3.5 mg, 2.75 mg to 3.5 mg, about 3 mg, about 3.25 mg, or about 3.3 mg) of polysorbate 20. In some of these embodiments, the tablet comprises 140 mg to 160 mg (e.g., 145 mg to 155 mg, 147 mg to 152 mg, about 148 mg, about 150 mg, or about 151 mg) of silicified microcrystalline cellulose. In some of these embodiments, the tablet comprises 2.5 mg to 8.5 mg (e.g., 4 mg to 8 mg, 5 mg to 7 mg, 6.25 mg to 6.75 mg, about 6.3 mg, about 6.4 mg, about or about 6.5 mg) of colloidal anhydrous silica. In some of these embodiments, the tablet comprises 25 mg to 40 mg (e.g., 27 mg to 37 mg, 30 mg to 35 mg, about 30 mg, about 32 mg, or about 33 mg) of microcrystalline cellulose. In some of these embodiments, the tablet comprises 25 mg to 40 mg (e.g., 27 mg to 37 mg, 30 mg to 35 mg, about 30 mg, about 32 mg, or about 33 mg) of partially or fully pregelatinized maize starch. In some of these embodiments, the tablet comprises 25 mg to 40 mg (e.g., 27 mg to 37 mg, 30 mg to 35 mg, about 30 mg, about 32 mg, or about 33 mg) of sodium stearyl fumarate.

In some embodiments, each tablet comprises an intragranular phase and an extragranular phase, the intragranular phase of the composition comprising (a) about 334 mg or about 335 mg (e.g., about 334.2 mg) of crystalline Compound (1) HCl hemihydrate salt (e.g., Compound (1) HCl hemihydrate crystalline salt Form A); (b) about 9 or about 10 mg (e.g., about 9.75 mg) of crospovidone; (c) about 12 mg or about 13 mg (e.g., about 12.35 mg) of hydroxypropyl methylcellulose having a viscosity of about 15 mPa·s; and (d) about 3.00 mg or about 4 mg (e.g., about 3.25 mg) of polysorbate 20; and the extragranular phase of the composition comprising (a) about 35.00 mg or about 36 mg (e.g., about 35.75 mg) of crospovidone; (b) about 6 mg or about 7 mg (e.g., about 6.5 mg) of colloidal anhydrous silica; (c) about 32 mg or about 33 mg (e.g., about 32.5 mg) of sodium stearyl fumarate; (d) about 150 mg or about 151 mg (e.g., about 150.70 mg) of silicified microcrystalline cellulose; (e) about 32 mg or about 33 mg (e.g., about 32.5 mg) of microcrystalline cellulose; and (f) about 32 mg or about 33 mg (e.g., about 32.5 mg) of starch.

The present invention also provides a pharmaceutical composition comprising a tablet, wherein the tablet comprises (a) a plurality of granules forming an intragranular phase of the composition, wherein the granules are produced by fluid bed granulation and comprise (i) about 51 wt % or about 52 wt % (e.g., about 51.42 wt %) of crystalline Compound (1) HCl hemihydrate salt (e.g., Compound (1) HCl hemihydrate crystalline salt Form A) by weight of the pharmaceutical composition, wherein Compound (1) is represented by the following structural formula:

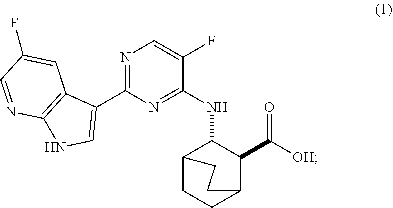

(ii) about 1 wt % or about 2 wt % (e.g., about 1.50 wt %) of crospovidone by weight of the pharmaceutical composition; (iii) about 1 wt % or about 2 wt % (e.g., about 1.90 wt %) of hydroxypropyl methylcellulose having a viscosity of about 15 mPa·s, by weight of the pharmaceutical composition; and (iv) about 0.50 wt % of polysorbate 20 by weight of the pharmaceutical composition; and (b) one or more excipients forming an extragranular phase of the composition, wherein the extragranular phase of the composition comprises (i) about 5 wt % or about 6 wt % (e.g., about 5.50 wt %) of crospovidone by weight of the pharmaceutical composition; (ii) about 1.00 wt % of colloidal anhydrous silica by weight of the pharmaceutical composition; (iii) about 5.00 wt % of sodium stearyl fumarate by weight of the pharmaceutical composition; (iv) about 23.18 wt % of silicified microcrystalline cellulose by weight of the pharmaceutical composition; (v) about 5.00 wt % of microcrystalline cellulose by weight of the pharmaceutical composition; and (vi) about 5.00 wt % of starch by weight of the pharmaceutical composition.

In some embodiments, each tablet comprises an intragranular phase and an extragranular phase, (a) the intragranular phase of the composition comprising (i) about 334.20 mg of crystalline Compound (1) HCl hemihydrate salt (e.g., Compound (1) HCl hemihydrate crystalline salt Form A); (ii) about 9.75 mg of crospovidone; (iii) about 12.35 mg of hydroxypropyl methylcellulose having a viscosity of about 15 mPa·s; and (iv) about 3.25 mg of polysorbate 20; (b) and the extragranular phase of the composition comprising (i) about 35.75 mg of crospovidone; (ii) about 6.5 mg of colloidal anhydrous silica; (iii) about 32.50 mg of sodium stearyl fumarate; (iv) about 150.70 mg of silicified microcrystalline cellulose; (v) about 32.50 mg of microcrystalline cellulose; and (vi) about 32.50 mg of starch.

The present invention also provides a pharmaceutical composition comprising a tablet, wherein the tablet comprises: a) a plurality of granules forming an intragranular phase of the composition, wherein the granules are produced by fluid bed granulation and comprise: (i) about 51.42 wt % of crystalline Compound (1) HCl hemihydrate salt (e.g., Compound (1) HCl hemihydrate crystalline salt Form A) by weight of the pharmaceutical composition, wherein Compound (1) is represented by the following structural formula:

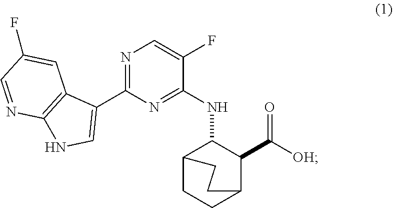

(ii) about 1.50 wt % of crospovidone by weight of the pharmaceutical composition; (iii) about 1.90 wt % of hydroxypropyl methylcellulose having a viscosity of about 15 mPa·s, by weight of the pharmaceutical composition; and (iv) about 0.50 wt % of polysorbate 20 by weight of the pharmaceutical composition; and b) one or more excipients forming an extragranular phase of the composition, wherein the extragranular phase of the composition comprises: (i) about 5.50 wt % of crospovidone by weight of the pharmaceutical composition; (ii) about 1.00 wt % of colloidal anhydrous silica by weight of the pharmaceutical composition; (iii) about 2.00 wt % of sodium stearyl fumarate by weight of the pharmaceutical composition; (iv) about 26.18 wt % of silicified microcrystalline cellulose by weight of the pharmaceutical composition; (v) about 5.00 wt % of microcrystalline cellulose by weight of the pharmaceutical composition; and (vi) about 5.00 wt % of starch by weight of the pharmaceutical composition.

In some embodiments, each tablet comprises an intragranular phase and an extragranular phase, the intragranular phase of the composition comprising: a) about 334.20 mg of crystalline Compound (1) HCl hemihydrate salt (e.g., Compound (1) HCl hemihydrate crystalline salt Form A); b) about 9.75 mg of crospovidone; c) about 12.35 mg of hydroxypropyl methylcellulose having a viscosity of about 15 mPa·s; and d) about 3.25 mg of polysorbate 20; and the extragranular phase of the composition comprising: a) about 35.75 mg of crospovidone; b) about 6.5 mg of colloidal anhydrous silica; c) about 13.00 mg of sodium stearyl fumarate; d) about 170.20 mg of silicified microcrystalline cellulose; e) about 32.50 mg of microcrystalline cellulose; and e) about 32.50 mg of starch.

The present invention also provides a process for producing a pharmaceutical composition comprising (a) mixing a binder and a wetting agent in water to form a substantially clear binder solution; (b) mixing crystalline Compound (1) HCl hemihydrate salt (e.g., Compound (1) HCl hemihydrate crystalline salt Form A) and a first disintegrant under heating conditions in a fluid bed granulizer to form a substantially homogenous mixture; (c) spraying the binder solution onto the homogenous mixture under fluidizing conditions to form a plurality wet granules; (d) drying the wet granules under fluidizing conditions to form dry granules; (e) mixing the dry granules and a glidant to form a substantially homogenous second mixture; (f) mixing a diluent, a second disintegrant, and the homogenous second mixture to form a substantially homogenous third mixture; (g) mixing a lubricant and the homogenous third mixture to form a substantially homogenous fourth mixture; and (h) compressing the homogenous fourth mixture into tablets using a tablet press.

In some implementations, the binder is hydroxypropyl methylcellulose (HPMC), and/or the wetting agent is polysorbate 20.

In some implementations, the first disintegrant is crospovidone. In some implementations, the second disintegrant is crospovidone. And, in some implementations, the first disintegrant and the second disintegrant is crospovidone.

In some implementations, the glidant is colloidal anhydrous silica.

In some implementations, the diluent comprises silicified microcrystalline cellulose, microcrystalline cellulose, pregelatinized starch, or any combination thereof. For example, the diluent comprises silicified microcrystalline cellulose and pregelatinized starch. In other examples, the diluent comprises silicified microcrystalline cellulose and microcrystalline cellulose.

In some implementations, the lubricant is sodium stearyl fumarate.

In some embodiments, the tablet press comprises one or more punches and one or more dies, and wherein the punches and dies of the tablet press are sprayed with a lubricant using an external lubrication system. In a further embodiment, the lubricant that is sprayed onto the punches and dies is sodium stearyl fumarate.

In one embodiment, the amount of lubricant sprayed onto the punches and dies is about 0.02 to about 0.25 wt % of the pharmaceutical composition. In a further embodiment, the amount of lubricant sprayed onto the punches and dies is about 0.05 wt % of the pharmaceutical composition.

Some implementations further comprise coating the tablet with a coating material, wherein the coating material comprises a polymer, plasticizer and pigment. In some examples, the film coating comprises a polymer, plasticizer, an anti-tacking agent, and pigment. In one embodiment, the anti-tacking agent is talc. In one embodiment, the pigment is titanium dioxide.

For tablet compositions of the invention, the methods further comprise film coating the tablet compositions. Typical film coating materials include one or more colorants, such as Opadry® II white or Opadry® II yellow.

Examples, including specific examples, of crystalline Compound (1) HCl hemihydrate salt (e.g., Compound (1) HCl hemihydrate crystalline salt Form A), diluents, disintegrant agents, binders, and lubricants, and modifiers which can be employed for the methods of preparing pharmaceutical compositions are each independently as described above for the pharmaceutical compositions of the invention.

The pharmaceutical compositions of the invention are pharmaceutically acceptable. As used herein, “pharmaceutically acceptable” means being inert without unduly inhibiting the biological activity of the active compound(s) (e.g., crystalline Compound (1) HCl hemihydrate salt (e.g., Compound (1) HCl hemihydrate crystalline salt Form A)), and biocompatible (e.g., non-toxic, non-inflammatory, non-immunogenic or devoid of other undesired reactions or side-effects upon the administration to a subject).

The pharmaceutical compositions of the invention may further include one or more pharmaceutically acceptable carriers other than those described above. The pharmaceutically acceptable carriers should be biocompatible. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as phosphates or glycine), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

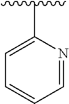 [SMILES: *c1ccccn1]

also represents

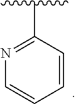 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium (D) analogs, can also be therapeutically useful.

The compounds described herein are defined herein by their chemical structures and/or chemical names. Where a compound is referred to by both a chemical structure and a chemical name, and the chemical structure and chemical name conflict, the chemical structure is determinative of the compound's identity.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds in accordance with the present invention are provided in the form of a single enantiomer at least 95%, at least 97% and at least 99% free of the corresponding enantiomer.

In a further embodiment, the compounds in accordance with the present invention are in the form of the (+) enantiomer at least 95% free of the corresponding (−) enantiomer.

In a further embodiment, the compounds in accordance with the present invention are in the form of the (+) enantiomer at least 97% free of the corresponding (−) enantiomer.

In a further embodiment, the compounds in accordance with the present invention are in the form of the (+) enantiomer at least 99% free of the corresponding (−) enantiomer.

Ina further embodiment, the compounds in accordance with the present invention are in the form of the (−) enantiomer at least 95% free of the corresponding (+) enantiomer.

In a further embodiment, the compounds in accordance with the present invention are in the form of the (−) enantiomer at least 97% free of the corresponding (+) enantiomer.

In a further embodiment the compounds in accordance with the present invention are in the form of the (−) enantiomer at least 99% free of the corresponding (+) enantiomer.

Another aspect of the present invention provides a packaged pharmaceutical composition, wherein the pharmaceutical composition is any composition described herein (e.g., a tablet) sealed in a blister pack, wherein the blister pack comprises a composition retaining layer configured to hold one or more pharmaceutical compositions (e.g., tablets) and a sealing layer configured to overlay the retaining layer to seal the pharmaceutical composition(s) within the retaining layer, wherein the sealing layer comprises aluminium foil and a desiccant material. As used herein, the term “desiccant material” refers to any hygroscopic substance useful as a drying agent. Examples of desiccant materials include without limitation silica (e.g., silica gel), activated charcoal, calcium sulfate, calcium chloride, and zeolite materials.

In some embodiments, the retaining layer comprises one or more chambers, wherein each chamber is configured to hold one or more pharmaceutical compositions (such as any pharmaceutical composition described herein (e.g., one or more tablets), and each chamber is sealed by the sealing layer. In some embodiments, the retaining layer comprises a clear or opaque material (e.g., a clear or opaque polyethylene material). In some embodiments, the sealing layer entirely overlaps the retaining layer and any chambers provided in the retaining layer.

Examples of commercially available blister packs useful for the present invention include Dessiflex Plus and Dessiflex Ultra available from Amcor plc. In some embodiments, the packaged pharmaceutical composition consists of 1 or more (e.g., 1, 2, 3, 4, 5, 6, 7, 8, 9, 10, 1 to 4, 2 to 10, or 1 to 10) tablets sealed in the blister pack, wherein the blister pack comprises one card. The stability and shelf life of the pharmaceutical compositions of the instant invention are improved using the blister pack packaging of the instant invention, wherein the sealing layer comprises a desiccant material, as compared to packaging the pharmaceutical composition in a blister pack having a sealing layer that lacks a desiccant material (e.g., Aclar 400 blister pack).

Another aspect of the present invention provides a kit comprising a packaged pharmaceutical composition, such as any packaged pharmaceutical composition described herein, and instructions for the administration of the packaged pharmaceutical composition.

### III. USE OF THE PHARMACEUTICAL COMPOSITION

One aspect of the present invention is generally related to the use of the pharmaceutically acceptable compositions described above, for inhibiting the replication of influenza viruses in a biological sample or in a patient, for reducing the amount of influenza viruses (reducing viral titer) in a biological sample or in a patient, and for treating influenza in a patient. Hereinafter unless specifically indicated otherwise, the various solid forms (e.g., polymorphs of HCl salts of Compound (1) or pharmaceutically acceptable salts thereof) described above are also referred to generally compounds.

In one embodiment, the present invention is generally related to the use of the compounds or pharmaceutical compositions disclosed herein (e.g., in pharmaceutically acceptable compositions) for any of the uses specified above.

In yet another embodiment, the pharmaceutical compositions disclosed herein can be used to reduce viral titre in a biological sample (e.g. an infected cell culture) or in humans (e.g. lung viral titre in a patient).

The terms “influenza virus mediated condition”, “influenza infection”, or “Influenza”, as used herein, are used interchangeable to mean the disease caused by an infection with an influenza virus.

Influenza is an infectious disease that affects birds and mammals caused by influenza viruses. Influenza viruses are RNA viruses of the family Orthomyxoviridae, which comprises five genera: Influenza virus A, Influenza virus B, Influenza virus C, ISA virus and Thogoto virus. Influenza virus A genus has one species, influenza A virus which can be subdivided into different serotypes based on the antibody response to these viruses: H1N1, H2N2, H3N2, H5N1, H7N7, H1N2, H9N2, H7N2, H7N3 and H10N7. Additional examples of influenza A virus include H3N8 and H7N9. Influenza virus B genus has one species, influenza B virus. Influenza B almost exclusively infects humans and is less common than influenza A. Influenza virus C genus has one species, Influenza virus C virus, which infects humans and pigs and can cause severe illness and local epidemics. However, Influenza virus C is less common than the other types and usually seems to cause mild disease in children.

In some embodiments of the invention, influenza or influenza viruses are associated with Influenza virus A or B. In some embodiments of the invention, influenza or influenza viruses are associated with Influenza virus A. In some specific embodiments of the invention, Influenza virus A is H1N1, H2N2, H3N2 or H5N1. In some specific embodiments of the invention, Influenza virus A is H1N1, H3N2, H3N8, H5N1, and H7N9. In some specific embodiments of the invention, Influenza virus A is H1N1, H3N2, H3N8, and H5N1.

In humans, common symptoms of influenza are chills, fever, pharyngitis, muscle pains, severe headache, coughing, weakness, and general discomfort. In more serious cases, influenza causes pneumonia, which can be fatal, particularly in young children and the elderly. Although it is often confused with the common cold, influenza is a much more severe disease and is caused by a different type of virus. Influenza can produce nausea and vomiting, especially in children, but these symptoms are more characteristic of the unrelated gastroenteritis, which is sometimes called “stomach flu” or “24-hour flu”.

Symptoms of influenza can start quite suddenly one to two days after infection. Usually the first symptoms are chills or a chilly sensation, but fever is also common early in the infection, with body temperatures ranging from 38-39° C. (approximately 100-103° F.). Many people are so ill that they are confined to bed for several days, with aches and pains throughout their bodies, which are worse in their backs and legs. Symptoms of influenza may include: body aches, especially joints and throat, extreme coldness and fever, fatigue, headache, irritated watering eyes, reddened eyes, skin (especially face), mouth, throat and nose, abdominal pain (in children with influenza B). Symptoms of influenza are non-specific, overlapping with many pathogens (“influenza-like illness). Usually, laboratory data is needed to confirm the diagnosis.

The terms, “disease”, “disorder”, and “condition” may be used interchangeably here to refer to an influenza virus mediated medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, cell cultures or extracts thereof; biopsied material obtained from a mammal or extracts thereof, blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the term “inhibition of the replication of influenza viruses” includes both the reduction in the amount of virus replication (e.g. the reduction by at least 10%) and the complete arrest of virus replication (i.e., 100% reduction in the amount of virus replication). In some embodiments, the replication of influenza viruses is inhibited by at least 50%, at least 65%, at least 75%, at least 85%, at least 90%, or at least 95%.

Influenza virus replication can be measured by any suitable method known in the art. For example, influenza viral titre in a biological sample (e.g. an infected cell culture) or in humans (e.g. lung viral titre in a patient) can be measured. More specifically, for cell based assays, in each case cells are cultured in vitro, virus is added to the culture in the presence or absence of a test agent, and after a suitable length of time a virus-dependent endpoint is evaluated. For typical assays, the Madin-Darby canine kidney cells (MDCK) and the standard tissue culture adapted influenza strain, A/Puerto Rico/8/34 can be used. A first type of cell assay that can be used in the invention depends on death of the infected target cells, a process called cytopathic effect (CPE), where virus infection causes exhaustion of the cell resources and eventual lysis of the cell. In the first type of cell assay, a low fraction of cells in the wells of a microtiter plate are infected (typically 1/10 to 1/1000), the virus is allowed to go through several rounds of replication over 48-72 hours, then the amount of cell death is measured using a decrease in cellular ATP content compared to uninfected controls. A second type of cell assay that can be employed in the invention depends on the multiplication of virus-specific RNA molecules in the infected cells, with RNA levels being directly measured using the branched-chain DNA hybridization method (bDNA). In the second type of cell assay, a low number of cells are initially infected in wells of a microtiter plate, the virus is allowed to replicate in the infected cells and spread to additional rounds of cells, then the cells are lysed and viral RNA content is measured. This assay is stopped early, usually after 18-36 hours, while all the target cells are still viable. Viral RNA is quantitated by hybridization to specific oligonucleotide probes fixed to wells of an assay plate, then amplification of the signal by hybridization with additional probes linked to a reporter enzyme.

As used herein a “viral titer (or titre)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. A specific example is viral titer. To determine the titer, several dilutions will be prepared, such as 10⁻¹, 10⁻², 10⁻³, 10⁻⁴, 10⁻⁵, 10⁻⁶, 10⁻⁷, 10⁻⁸, or the like. The lowest concentration of virus that still infects cells is the viral titer.

As used herein, the terms “treat”, “treatment” and “treating” refer to both therapeutic and prophylactic treatments. For example, therapeutic treatments include the reduction or amelioration of the progression, severity and/or duration of influenza viruses mediated conditions, or the amelioration of one or more symptoms (specifically, one or more discernible symptoms) of influenza viruses mediated conditions, resulting from the administration of one or more therapies (e.g., one or more therapeutic agents such as a compound or composition of the invention). In specific embodiments, the therapeutic treatment includes the amelioration of at least one measurable physical parameter of an influenza virus mediated condition. In other embodiments the therapeutic treatment includes the inhibition of the progression of an influenza virus mediated condition, either physically by, e.g., stabilization of a discernible symptom, physiologically by, e.g., stabilization of a physical parameter, or both. In other embodiments the therapeutic treatment includes the reduction or stabilization of influenza viruses mediated infections. Antiviral drugs can be used in the community setting to treat people who already have influenza to reduce the severity of symptoms and reduce the number of days that they are sick.

The term “chemotherapy” refers to the use of medications, e.g. small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious influenza complications live in close contact with each other (e.g. in a hospital ward, daycare center, prison, nursing home, etc.). It also includes the use among populations who require protection from the influenza but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the influenza or not considered at high risk for complications, in order to reduce the chances of getting infected with the influenza and passing it on to a high-risk person in close contact with him (for instance, healthcare workers, nursing home workers, etc.).

According to the US CDC, an influenza “outbreak” is defined as a sudden increase of acute febrile respiratory illness (AFRI) occurring within a 48 to 72 hour period, in a group of people who are in close proximity to each other (e.g. in the same area of an assisted living facility, in the same household, etc.) over the normal background rate or when any subject in the population being analyzed tests positive for influenza. One case of confirmed influenza by any testing method is considered an outbreak.

A “cluster” is defined as a group of three or more cases of AFRI occurring within a 48 to 72 hour period, in a group of people who are in close proximity to each other (e.g. in the same area of an assisted living facility, in the same household, etc.).

As used herein, the “index case”, “primary case” or “patient zero” is the initial patient in the population sample of an epidemiological investigation. When used in general to refer to such patients in epidemiological investigations, the term is not capitalized. When the term is used to refer to a specific person in place of that person's name within a report on a specific investigation, the term is capitalized as Patient Zero. Often scientists search for the index case to determine how the disease spread and what reservoir holds the disease in between outbreaks. Note that the index case is the first patient that indicates the existence of an outbreak. Earlier cases may be found and are labeled primary, secondary, tertiary, etc.

In one embodiment, the methods of the invention are a preventative or “pre-emptive” measure to a patient, specifically a human, having a predisposition to complications resulting from infection by an influenza virus. The term “pre-emptive” as used herein as for example in pre-emptive use, “pre-emptively”, etc., is the prophylactic use in situations in which an “index case” or an “outbreak” has been confirmed, in order to prevent the spread of infection in the rest of the community or population group.

In another embodiment, the methods of the invention are applied as a “pre-emptive” measure to members of a community or population group, specifically humans, in order to prevent the spread of infection.

As used herein, an “effective amount” refers to an amount sufficient to elicit the desired biological response. In the present invention the desired biological response is to inhibit the replication of influenza virus, to reduce the amount of influenza viruses or to reduce or ameliorate the severity, duration, progression, or onset of a influenza virus infection, prevent the advancement of an influenza viruses infection, prevent the recurrence, development, onset or progression of a symptom associated with an influenza virus infection, or enhance or improve the prophylactic or therapeutic effect(s) of another therapy used against influenza infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other antiviral agents, e.g., when co-administered with an anti-influenza medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, the compounds disclosed herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds described herein can range from 0.01 to 100 mg/kg body weight/day, 0.01 to 50 mg/kg body weight/day, 0.1 to 50 mg/kg body weight/day, or 1 to 25 mg/kg body weight/day. It is understood that the total amount per day can be administered in a single dose or can be administered in multiple dosing, such as twice a day (e.g., every 12 hours), three times a day (e.g., every 8 hours), or four times a day (e.g., every 6 hours).

In some embodiments, dosages of the compounds described herein (e.g., Compound (1) and its pharmaceutically acceptable salts and hydrates thereof, including Compound (1) HCl hemihydrate salt Form A) are in a range of 100 mg to 1,600 mg, such as 400 mg to 1,600 mg or 400 mg to 1,200 mg. Each dose can be taken once a day (QD), twice per day (e.g., every 12 hours (BID)), or three times per day (e.g., q8h (TID)). It is noted that any combinations of QD, BID, and TID can be employed, as desired, such as BID on day 1, followed by QD thereafter, or, when a loading dosage is employed on day 1, BID on day 2, followed by QD thereafter.

In one specific embodiment, dosages of the compounds described herein are 400 mg to 1,600 mg, 400 mg to 1,200 mg, or 600 mg to 1,200 mg once a day. In another specific embodiment, dosages of the compounds described herein are 400 mg to 1,600 mg, 400 mg to 1,200 mg, or 300 mg to 900 mg twice a day. In yet another specific embodiment, dosages of the compounds described herein are 400 mg to 1,000 mg once a day. In yet another specific embodiment, dosages of the compounds described herein are 600 mg to 1,000 mg once a day. In yet another specific embodiment, dosages of the compounds described herein are 600 mg to 800 mg once a day. In yet another specific embodiment, dosages of the compounds described herein are 400 mg to 800 mg twice a day (e.g., 400 mg to 800 mg every 12 hours). In yet another specific embodiment, dosages of the compounds described herein are 400 mg to 600 mg twice a day.

In one embodiment, dosages of the compounds described herein are 250 mg to 350 mg (e.g. 300 mg) or 550 mg to 650 mg (e.g. 600 mg) once a day. In another specific embodiment, dosages of the compounds described herein are 250 mg to 350 mg (e.g. 300 mg) or 550 mg to 650 mg (e.g. 600 mg) twice a day. In another embodiment, the dosage of the compounds described herein is 600 mg twice a day. In a further embodiment, the dosage of the compounds described herein is the administration of two 300 mg tablets twice per day (bid), i.e., 600 mg twice per day, for a total of 1200 mg per day. In some embodiments, the dose weight refers to the dose weight of the free base of Compound (1) or the free base equivalent weight of Compound (1) where a salt and/or hydrate of Compound (1) (e.g., Compound (1) HCl hemihydrate (e.g., Compound (1) HCl hemihydrate crystalline salt Form A)) is used to prepare the dosage form.

In some embodiments, a loading dosage regimen is employed. In one specific embodiment, a loading dose of 400 mg to 1,600 mg is employed on day 1 of treatment. In another specific embodiment, a loading dose of 600 mg to 1,600 mg is employed on day 1 of treatment. In another specific embodiment, a loading dose of 800 mg to 1,600 mg is employed on day 1 of treatment. In yet another specific embodiment, a loading dose of 900 mg to 1,600 mg is employed on day 1 of treatment. In yet another specific embodiment, a loading dose of 900 mg to 1,200 mg is employed on day 1 of treatment. In yet another specific embodiment, a loading dose of 900 mg is employed on day 1 of treatment. In yet another specific embodiment, a loading dose of 1,000 mg is employed on day 1 of treatment. In yet another specific embodiment, a loading dose of 1,200 mg is employed on day 1 of treatment.

In one specific embodiment, the dosage regimen of the compounds described herein employs a loading dosage of 600 mg to 1,600 mg on day 1 and with a regular dosage of 300 mg to 1,200 mg for the rest of the treatment duration. Each regular dose can be taken once a day, twice a day, or three times a day, or any combination thereof. In a further specific embodiment, a loading dosage of 900 mg to 1,600 mg, such as 900 mg, 1,200 mg, or 1,600 mg, is employed. In another further specific embodiment, a loading dosage of 900 mg to 1,200 mg, such as 900 mg or 1,200 mg, is employed. In yet another further specific embodiment, a regular dosage of 400 mg to 1,200 mg, such as 400 mg, 600 mg, or 800 mg, is employed for the rest of the treatment duration. In yet another further specific embodiment, a regular dosage of 400 mg to 1,000 mg for the rest of the treatment duration. In yet another further specific embodiment, a regular dosage of 400 mg to 800 mg is employed for the rest of the treatment duration. In yet another further specific embodiment, a regular dosage of 300 mg to 900 mg twice a day is employed. In yet another further specific embodiment, a regular dosage of 600 mg to 1,200 mg once a day is employed. In yet another further specific embodiment, a regular dosage of 600 mg twice a day on day 2, followed by 600 mg once a day for the rest of the treatment duration.

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). Alternatively, for therapeutic treatment, the compounds described herein can be administered to a patient within, for example, 96 hours of onset of symptoms. The therapeutic treatment can last for any suitable duration, for example, for 3 days, 4 days, 5 days, 7 days, 10 days, 14 days, etc. For prophylactic treatment during a community outbreak, the compounds described herein can be administered to a patient within, for example, 2 days of onset of symptoms in the index case, and can be continued for any suitable duration, for example, for 7 days, 10 days, 14 days, 20 days, 28 days, 35 days, 42 days, etc., up to the entire flu season. A flu season is an annually-recurring time period characterized by the prevalence of outbreaks of influenza. Influenza activity can sometimes be predicted and even tracked geographically. While the beginning of major flu activity in each season varies by location, in any specific location these minor epidemics usually take 3-4 weeks to peak and another 3-4 weeks to significantly diminish. Typically, Centers for Disease Control (CDC) collects, compiles and analyzes information on influenza activity year round in the United States and produces a weekly report from October through mid-May.

In one embodiment, the therapeutic treatment lasts for 1 day to an entire flu season. In one specific embodiment, the therapeutic treatment lasts for 3 days to 14 days. In another specific embodiment, the therapeutic treatment lasts for 5 days to 14 days. In another specific embodiment, the therapeutic treatment lasts for 3 days to 10 days. In yet another specific embodiment, the therapeutic treatment lasts for 4 days to 10 days. In yet another specific embodiment, the therapeutic treatment lasts for 5 days to 10 days. In yet another specific embodiment, the therapeutic treatment lasts for 4 days to 7 days (e.g., 4 days, 5 days, 6 days, or 7 days). In yet another specific embodiment, the therapeutic treatment lasts for 5 days to 7 days (e.g., 5 days, 6 days, or 7 days). In one specific embodiment, the prophylactic treatment lasts up to the entire flu season.

In one specific embodiment, the compounds described herein are administered to a patient for 3 days to 14 days (e.g., 5 days to 14 days) with a loading dosage of 900 mg to 1,600 mg on day 1 and with a regular dosage of 300 mg to 1,200 mg for the rest of the treatment duration. In another specific embodiment, the compounds described herein are administered to a patient for 3 days to 14 days (e.g., 5 days to 14 days) with a loading dosage of 900 mg to 1,200 mg on day 1 and with a regular dosage of 400 mg to 1,000 mg for the rest of the treatment duration. In yet another specific embodiment, the compounds described herein are administered to a patient for 3 days to 14 days (e.g., 5 days to 14 days) with a loading dosage of 900 mg to 1,200 mg on day 1 and with a regular dosage of 400 mg to 800 mg for the rest of the treatment duration. In yet another specific embodiment, the compounds described herein are administered to a patient for 3 days to 14 days (e.g., 5 days to 14 days) with a loading dosage of 900 mg to 1,200 mg on day 1 and with a regular dosage of 400 mg to 800 mg for the rest of the treatment duration. Each dose can be taken once a day, twice a day, or three times a day, or any combination thereof.

In one specific embodiment, the compounds described herein are administered to a patient for 3 days to 14 days with a loading dosage of 900 mg to 1,600 mg on day 1 and with a regular dosage of 600 mg to 1,000 mg once a day for the rest of the treatment duration. In another specific embodiment, the compounds described herein are administered to a patient for 3 days to 14 days with a loading dosage of 900 mg to 1,200 mg on day 1 and with a regular dosage of 600 mg to 800 mg (e.g., 600 mg, 650 mg, 700 mg, 750 mg, or 800 mg) once a day for the rest of the treatment duration. In some embodiments, the treatment duration is for 4 days to 10 days, 5 days to 10 days, or 5 days to 7 days.

In one specific embodiment, the compounds described herein are administered to a patient for 3 days to 14 days with a loading dosage of 900 mg to 1,600 mg on day 1 and with a regular dosage of 400 mg to 800 mg twice a day for the rest of the treatment duration. In another specific embodiment, the compounds described herein are administered to a patient for 3 days to 14 days with a loading dosage of 900 mg to 1,200 mg on day 1 and with a regular dosage of 400 mg to 600 mg (e.g., 400 mg, 450 mg, 500 mg, 550 mg, or 600 mg) twice a day for the rest of the treatment duration. In some embodiments, the duration is for 4 days to 10 days, 5 days to 10 days, or 5 days to 7 days.

In one specific embodiment, the compounds described herein are administered to a patient for 4 days or 5 days with a loading dosage of 900 mg to 1,200 mg (e.g., 900 mg or 1,200 mg) on day 1 and with a regular dosage of 400 mg to 600 mg (e.g., 400 mg or 600 mg) twice a day for the rest of the treatment duration (e.g., days 2 through 4, or days 2 through 5). In another specific embodiment, the compounds described herein are administered to a patient for 4 days or 5 days with a loading dosage of 900 mg to 1,200 mg (e.g., 900 mg or 1,200 mg) on day 1 and with a regular dosage of 600 mg to 800 mg (e.g., 600 mg or 800 mg) once a day for the rest of the treatment duration.

Various types of administration methods can be employed in the invention, and are described in detail below under the section entitled “Administration Methods”.

### IV. COMBINATION THERAPY

An effective amount can be achieved in the method or pharmaceutical composition of the invention employing a compound of the invention (including a pharmaceutically acceptable salt or solvate (e.g., hydrate)) alone or in combination with an additional suitable therapeutic agent, for example, an antiviral agent or a vaccine. When “combination therapy” is employed, an effective amount can be achieved using a first amount of a compound of the invention and a second amount of an additional suitable therapeutic agent (e.g., an antiviral agent or vaccine) administered in any order or concurrently.

In another embodiment of this invention, a compound of the invention and the additional therapeutic agent, are each administered in an effective amount (i.e., each in an amount which would be therapeutically effective if administered alone). In another embodiment, a compound of the invention and the additional therapeutic agent, are each administered in an amount which alone does not provide a therapeutic effect (a sub-therapeutic dose). In yet another embodiment, a compound of the invention can be administered in an effective amount, while the additional therapeutic agent is administered in a sub-therapeutic dose. In still another embodiment, a compound of the invention can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

In one embodiment, the present invention is directed to methods of combination therapy for inhibiting Flu viruses replication in biological samples or patients, or for treating or preventing Influenza virus infections in patients using the compounds described herein. Accordingly, pharmaceutical compositions of the invention also include those comprising an inhibitor of Flu virus replication of this invention in combination with an anti-viral compound exhibiting anti-Influenza virus activity.

Methods of use of the compounds described herein and compositions of the invention also include combination of chemotherapy with a compound or composition of the invention, or with a combination of a compound or composition of this invention with another anti-viral agent and vaccination with a Flu vaccine.

When co-administration involves the separate administration of the first amount of a compound of the invention and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of the invention and the second therapeutic agent can be administered in any order within 24 hours of each other, within 16 hours of each other, within 8 hours of each other, within 4 hours of each other, within 1 hour of each other or within 30 minutes of each other.

More, specifically, a first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

It is understood that the method of co-administration of a first amount of a compound of the invention and a second amount of an additional therapeutic agent can result in an enhanced or synergistic therapeutic effect, wherein the combined effect is greater than the additive effect that would result from separate administration of the first amount of a compound of the invention and the second amount of an additional therapeutic agent.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using the compounds of the present invention is in combination with a Flu vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

Specific examples that can be co-administered with a compound described herein include neuraminidase inhibitors, such as oseltamivir (Tamiflu®) and Zanamivir (Rlenza®), viral ion channel (M2 protein) blockers, such as amantadine (Symmetrel®) and rimantadine (Flumadine®), and antiviral drugs described in WO 2003/015798, including T-705 under development by Toyama Chemical of Japan. (See also Ruruta et al., Antiviral Research, 82: 95-102 (2009), “T-705 (favipiravir) and related compounds: Novel broad-spectrum inhibitors of RNA viral infections”). In some embodiments, the compounds described herein can be co-administered with a traditional influenza vaccine. In some embodiments, the compounds described herein can be co-administered with zanamivir. In some embodiments, the compounds described herein can be co-administered with oseltamivir. In some embodiments, the compounds described herein can be co-administered with favipiravir (T-705). In some embodiments, the compounds described herein can be co-administered with amantadine or rimantadine. Oseltamivir can be administered in a dosage regimen according to its label. In some specific embodiments, it is administered 75 mg twice a day, or 150 mg once a day.

Administration Methods

The compounds and pharmaceutically acceptable compositions described above can be administered to humans and other animals orally, rectally, intracisternally, intravaginally, intraperitoneally, topically (as by powders, ointments, or drops), buccally, as an oral or nasal spray, or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) diluents or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as diluents in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as diluents in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions described herein may be administered orally, by inhalation spray, topically, rectally, nasally, buccally, vaginally or via an implanted reservoir.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., 1 to 4 or more times per day). When multiple daily doses are used, the unit dosage form can be the same or different for each dose.

### V. EXAMPLES

General Methods of XRPD, ¹³C Solid State NMR, DSC, and TGA Measurements

Analytical Method 1A: Thermogravimetric Analysis (TGA)

Thermogravimetric analysis (TGA) was performed on the TA Instruments TGA model Q500 Asset Tag V014840. The solid sample was placed in a platinum sample pan and heated at 10° C./min to 300° C. from room temperature.

Analytical Method 1B: DSC Measurements

DSC was conducted on a TA Instruments DSC Q200 Asset Tag V015553. Approximately 1-2 mg of solid sample was placed in an aluminum hermetic DSC pan with a crimped lid with a pinhole. The sample cell was generally heated under nitrogen purge.

Analytical Method 1C: SSNMR experimental:

Solid state nuclear magnetic spectroscopy (SSNMR) spectra were acquired on the Bruker-Biospin 400 MHz Advance III wide-bore spectrometer equipped with Bruker-Biospin 4 mm HFX probe. Samples were packed into 4 mm ZrO₂ rotors (approximately 70 mg or less, depending on sample availability). Magic angle spinning (MAS) speed of typically 12.5 kHz was applied. The temperature of the probe head was set to 275K to minimize the effect of frictional heating during spinning. The proton relaxation time was measured using ¹H MAS Ti saturation recovery relaxation experiment in order to set up proper recycle delay of the ¹³C cross-polarization (CP) MAS experiment. The recycle delay of ¹³C CPMAS experiment was adjusted to be at least 1.2 times longer than the measured ¹H Ti relaxation time in order to maximize the carbon spectrum signal-to-noise ratio. The CP contact time of ¹³C CPMAS experiment was set to 2 ms. A CP proton pulse with linear ramp (from 50% to 100%) was employed. The Hartmann-Hahn match was optimized on external reference sample (glycine). Fluorine spectra were acquired using proton decoupled MAS setup with recycled delay set to approximately 5 times of the measured ¹⁹F Ti relaxation time. The fluorine relaxation time was measured using proton decoupled ¹⁹F MAS Ti saturation recovery relaxation experiment. Both carbon and fluorine spectra were acquired with SPINAL 64 decoupling was used with the field strength of approximately 100 kHz. The chemical shift was referenced against external standard of adamantane with its upfield resonance set to 29.5 ppm.

Analytical Method 1D: Bruker D8 Discover XRPD Experimental Details.

The XRPD patterns were acquired at room temperature in reflection mode using a Bruker D8 Discover diffractometer (Asset Tag V012842) equipped with a sealed tube source and a Hi-Star area detector (Bruker AXS, Madison, Wis.). The X-Ray generator was operating at a voltage of 40 kV and a current of 35 mA. The powder sample was placed in an aluminum holder. Two frames were registered with an exposure time of 120 s each. The data were subsequently integrated over the range of 4.5°-39° 2° with a step size of 0.02° and merged into one continuous pattern.

### Example 1: Preparation of Compound (1) and 2-MeTHF Solvate of Compound (1)

Compound (1) can be prepared as described in WO 2010/148197. For example, an amorphous free base of Compound (1) was prepared according to WO 2010/148197, followed by usual chiral separation and purification: SCF chiral chromatography with a modifier that included Et₂NH (which generated Et₂NH salt of Compound (1)) and then ion-exchange resin treatment. Alternatively, Compound (1) can be made by the following procedures as a 2-MeTHF solvate:

### Preparation of Compound 2a (2-Amino-3-bromo-5-fluoropyridine)

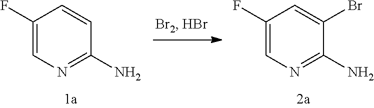 [SMILES: Nc1ccc(F)cn1 ; Nc1ncc(F)cc1Br]

To a slurry of 2-amino-5-fluoropyridine (6 kg, 53.6 mol) in water (24 L) at 14° C. was added over 10 minutes 48% hydrobromic acid (18.5 kg, 110 mol). The reaction was exothermic and the temperature went up to 24° C. The mixture was re-cooled to 12° C. then bromine (9 kg, 56.3 mol) was added in nine portions over 50 minutes (exothermic, kept at 20° C.). The mixture was stirred at 22° C. overnight, and monitored by ¹H NMR of a quenched aliquot (quenched 5 drops in to mix of 1 mL 20% K₂CO₃, 0.3 mL 10% Na₂S₂O₃ and 0.7 mL DCM. Organic layer evaporated and assayed). The mixture was cooled to 10° C. then quenched by addition of sodium bisulfite (560 g, 5.4 mol) in water (2 L), and further cooled to 0° C. This mixture was added to a cold (−4° C.) mixture of DCM (18 L) and 5.4 M sodium hydroxide (35 L, 189 mol). The bottom ˜35 L was filtered through a pad of Celite and then the phase break was made. The aqueous layer was re-extracted with DCM (10 L). The organics were filtered through a pad of 3 kg magnesol, washing with DCM (8 L). The filtrate was evaporated, triturated with hexane and filtered.

Despite the in-process assay indicating 97% completion, this initial product from all four runs typically contained 10% SM. These were combined and triturated in hexane (2 L per kg material) at 50° C., then cooled to 15° C. and filtered to afford Compound 2a (30.0 kg, 95% purity, 149 mol, 67%). Mother liquors from the initial trituration and the re-purification were chromatographed (20 kg silica, eluent 25-50% EtOAc in hexane) to afford additional Compound 2a (4.7 kg, 99% purity, 24.4 mol, 11%).

### Preparation of Compound 3a

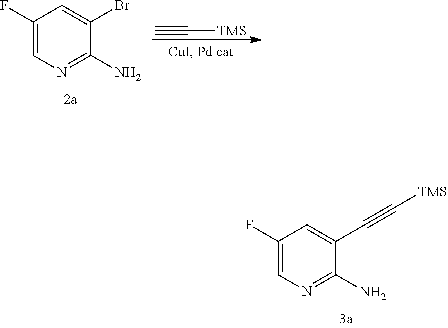

To an inert 400-L reactor was charged 2a (27.5 kg, 96% purity, 138 mol), Pd(PPh₃)₄ (1044 g, 0.90 mol) and CuI (165 g, 0.87 mol), followed by toluene (90 kg). The mixture was de-oxygenated with three vacuum-nitrogen cycles, then triethylamine (19.0 kg, 188 mol) was added. The mixture was de-oxygenated with one more vacuum-nitrogen cycle, then TMS-acetylene (16.5 kg, 168 mol) was added. The mixture was heated to 48° C. for 23 hours (the initial exotherm took the temperature to 53° C. maximum), then cooled to 18° C. The slurry was filtered through a pad of Celite and washed with toluene (80 kg). The filtrate was washed with 12% Na₂HPO₄ (75 L), then filtered through a pad of silica (25 kg), washing with 1:1 hexane:MTBE (120 L). This filtrate was evaporated to a brown oil and then dissolved in NMP for the next step. Weight of a solution of Compound 3a—58 kg, ˜50 wt %, 138 mol, 100%. ¹H NMR (CDCl₃, 300 MHz): δ 7.90 (s, 1H); 7.33-7.27 (m, 1H); 4.92 (s, NH₂), 0.28 (s, 9H) ppm.

### Preparation of Compound 4a

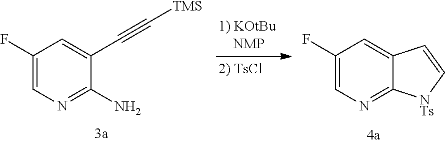 [SMILES: C[Si](C)(C)C#Cc1cc(F)cnc1N ; Cc1ccc(S(=O)(=O)n2ccc3cc(F)cnc32)cc1]

To an inert 400-L reactor was charged potassium t-butoxide (17.5 kg, 156 mol) and NMP (45 kg). The mixture was heated to 54° C. then a solution of Compound 3a (29 kg, 138 mol) in NMP (38 kg) was added over 2.75 hours and rinsed in with NMP (6 kg) (exothermic, maintained at 70-77° C.). The reaction was stirred at 74° C. for 2 hours then cooled to 30° C. and a solution of tosyl chloride (28.5 kg, 150 mol) in NMP (30 kg) added over 1.5 hours and rinsed in with NMP (4 kg). The reaction was exothermic and maintained at 30-43° C. The reaction was stirred for 1 hour while cooling to 20° C. then water (220 L) was added over 35 minutes (exothermic, maintained at 18-23° C.). The mixture was stirred at 20° C. for 30 minutes then filtered and washed with water (100 L). The solids were dissolved off the filter with DCM (250 kg), separated from residual water and the organics filtered through a pad of magnesol (15 kg, top) and silica (15 kg, bottom), washing with extra DCM (280 kg). The filtrate was concentrated to a thick slurry (˜50 L volume) then MTBE (30 kg) was added while continuing the distillation at constant volume (final distillate temperature of 51° C.). Additional MTBE (10 kg) was added and the slurry cooled to 15° C., filtered and washed with MTBE (40 L) to afford Compound 4a (19.13 kg, 95% purity, 62.6 mol, 45%). Partial concentration of the filtrate afforded a second crop (2.55 kg, 91% purity, 8.0 mol, 6%). ¹H NMR (CDCl₃, 300 MHz): δ 8.28-8.27 (m, 1H); 8.06-8.02 (m, 2H); 7.77 (d, J=4.0 Hz, 1H); 7.54-7.50 (m, 1H); 7.28-7.26 (m, 2H); 6.56 (d, J=4.0 Hz, 1H); 2.37 (s, 3H) ppm.

### Preparation of Compound 5a

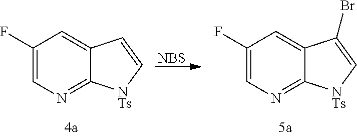

To a slurry of N-bromosuccinimide (14.16 kg, 79.6 mol) in DCM (30 kg) at 15° C. was charged a solution of Compound 4a (19.13 kg, 95% purity, and 2.86 kg, 91% purity, 71.6 mol) in DCM (115 kg), rinsing in with DCM (20 kg). The mixture was stirred at 25° C. for 18 hours, and then cooled to 9° C. and quenched by addition of a solution of sodium thiosulfate (400 g) and 50% sodium hydroxide (9.1 kg) in water (130 L). The mixture was warmed to 20° C. and the layers were separated and the organics were washed with 12% brine (40 L). The aqueous layers were sequentially re-extracted with DCM (4×50 kg). The organics were combined and 40 L distilled to azeotrope water, then the solution was filtered through a pad of silica (15 kg, bottom) and magensol (15 kg, top), washing with DCM (180 kg). The filtrate was concentrated to a thick slurry (˜32 L volume) then hexane (15 kg) was added. Additional hexane (15 kg) was added while continuing the distillation at constant volume (final distillate temperature 52° C.). The slurry was cooled to 16° C., filtered and washed with hexane (25 kg) to afford Compound 5a (25.6 kg, 69.3 mol, 97%). ¹H NMR (CDCl₃, 300 MHz): δ 8.34-8.33 (m, 1H); 8.07 (d, J=8.2 Hz, 2H); 7.85 (s, 1H); 7.52-7.49 (m, 1H); 7.32-7.28 (m, 2H); 2.40 (s, 3H) ppm.

### Preparation of Compound 6a: BEFTA1 Reaction

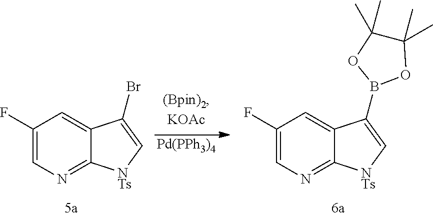 [SMILES: Cc1ccc(S(=O)(=O)n2cc(Br)c3cc(F)cnc32)cc1 ; Cc1ccc(S(=O)(=O)n2cc(B3OC(C)(C)C(C)(C)O3)c3cc(F)cnc32)cc1 ; "Pd(PPh3)4"]

To an inert 400-L reactor was charged Compound 5a (25.6 kg, 69.3 mol), bis(pinacolato)diboron (19 kg, 74.8 mol), potassium acetate (19 kg, 194 mol), palladium acetate (156 g, 0.69 mol) and triphenylphosphine (564 g, 2.15 mol), followed by dioxane (172 kg), that had been separately de-oxygenated using vacuum-nitrogen cycles (×3). The mixture was stirred and de-oxygenated using vacuum-nitrogen cycles (×2), then heated to 100° C. for 15 hours. The mixture was cooled to 35° C. then filtered, washing with 30° C. THF (75 kg). The filtrate was evaporated and the residue dissolved in DCM (˜90 L). The solution was stirred with 1 kg carbon and 2 kg magnesol for 45 minutes then filtered through a pad of silica (22 kg, bottom) and magensol (10 kg, top), washing with DCM (160 kg). The filtrate was concentrated to a thick slurry (˜40 L volume) then triturated at 35° C. and hexane (26 kg) was added. The slurry was cooled to 20° C., filtered and washed with a mix of DCM (5.3 kg) and hexane (15 kg), then hexane (15 kg) and dried under nitrogen on the filter to afford Compound 6a (23.31 kg, 56.0 mol, 81%) as a white solid. ¹H-NMR consistent with desired product, HPLC 99.5%, palladium assay 2 ppm. ¹H NMR (CDCl₃, 300 MHz): δ 8.25 (s, 1H); 8.18 (s, 1H); 8.09-8.02 (m, 2H); 7.91-7.83 (m, 1H); 7.30-7.23 (m, 2H); 2.39 (s, 3H); 1.38 (s, 12H) ppm.

### Preparation of Compounds 8a and 9a

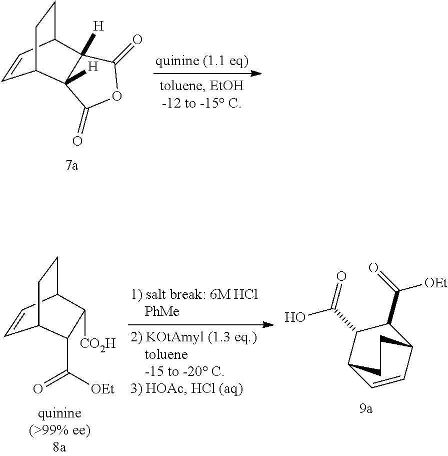

Compound 8a:

Anhydride 7a (24.6 kgs, Apex) and quinine (49.2 kgs, Buchler) were added to a reactor followed by the addition of anhydrous PhMe (795.1 kgs). The reactor was then cooled to −16° C. and EtOH (anhydrous, 41.4 kgs) was added at such a rate to maintain the internal reactor temperature <−12° C. The maximum reaction temp recorded for this experiment was −16° C. The reaction mixture was then stirred for 16 h at −16° C. A sample was removed and filtered. The solid was dried and evaluated by H-NMR which showed that no anhydride remained. The contents of the reactor were filtered. The reactor and subsequent wet cake were washed with PhMe (anhydrous, 20 kgs). The resulting solid was placed in a tray dryer at <45° C. with a N₂ sweep for at least 48 h. In this experiment, the actual temperature was 44° C. and the vacuum was −30 inHG. Material was sampled after 2.5 d drying and showed 3% PhMe by NMR. After an additional 8 hrs, the amt of PhMe analyzed showed the same 3% PhMe present and the drying was stopped. The weight of the white solid was 57.7 kgs, 76% yield. ¹H NMR showed consistent with structure and Chiral SFC analysis showed material >99% ee.

Compound 9a:

The reactor was charged with quinine salt 8a (57.7 kgs) and PhMe (250.5 kgs, Aldrich ACS grade, >99.5%) and the agitator was started. The contents were cooled to <15° C. and was treated with 6N HCl (18 kgs H₂O were treated with 21.4 kgs of conc. HCl) while keeping the temperature <25° C. The mixture was stirred for 40 min and visually inspected to verify that no solids were present. Stirring was stopped and the phases were allowed to settle and phases were separated. The aqueous phases were extracted again with PhMe (160 kgs; the amount typically used was much less, calc. 43 kgs. However, for efficient stirring due to minimal volume, additional PhMe was added. The organic phases were combined.

To the organic phases were cooled to <5° C. (0-5° C.) and was added sodium sulfate (anhydrous, 53.1 kgs) with agitation for 8 hrs (in this instance 12 hrs). The contents of the reactor containing the organic phase were passed through a filter containing sodium sulfate (31 kgs, anhydrous) and into a cleaned and dried reactor. The reactor was rinsed with PhMe (57.4 kgs), passed through the filter into reactor 201. The agitator was started and an additional amount of PhMe (44 kgs) was added and the reaction mixture cooled to −20° C. At that temperature PhMe solution of potassium tert-pentoxide was added over 2 h while keeping the temperature between −15 and −22° C. The reaction mixture was held at −20° C. for an additional 30 min before being sampled. Sampling occurred by removing an aliquot with immediate quenching into 6N HCl. The target ratio here is 96:4 (trans:cis).

Having achieved the target ratio, the reactor was charged with acetic acid (2.8 kgs) over 6 min. The temperature stayed at −20° C. The temperature was then adjusted to −5° C. and aqueous 2N HCl (65.7 kgs water treated with 15.4 kgs of conc. HCl) was added. The contents were warmed to 5° C.+/−5° C., agitated for 45 min before warming to 20° C.+/−5° C. with stirring for 15 min. The agitator was stopped and the phases allowed to settle. The aqueous layer was removed (temporary hold). The organic phase was washed with water (48 kgs, potable), agitated for 15 min and phases allowed to settle (at least 15 min) and the aqueous layer was removed and added to the aqueous layer. ⅓ of a buffer solution (50 L) that was prepared (7.9 kgs NaH₂PO₄, 1.3 kgs of Na₂HIPO₄ and 143.6 kgs water) was added to the organic phase and stirred for at least 15 min. Agitation was stopped and phases were allowed to separate for at least 15 min. The lower layer was discarded. Another portion of the buffered solution (50 L) was used to wash the organic layer as previously described. The wash was done a third time as described above.

Vacuum distillation of the PhMe phase (150 L) was started at 42° C./−13.9 psig and distilled to an oil of 20 L volume. After substantial reduction in volume the mixture was transferred to a smaller vessel to complete the distillation. Heptanes (13.7 kgs) was added and the mixture warmed to 40+/−5° C. for 30 min then the contents were cooled to 0-5° C. over 1.5 h. The solids were filtered and the reactor washed with approximately 14 kgs of cooled (0-5° C.) heptanes. The solids were allowed to dry under vacuum before placing in the oven at <40° C. under house vac (˜28 psig) until LOD is <1%. 15.3 kgs, 64%, 96% HPLC purity. ¹H NMR (400 MHz, CDCl₃) δ 11.45 (br. s, 1H), 6.41 (t, J=7.2 Hz, 1H), 6.25 (t, J=7.2 Hz, 1H), 4.18 (m, 2H), 3.27 (m, 1H), 3.03 (m, 1H), 2.95 (m, 1H), 2.77 (m, 1H), 1.68 (m, 1H), 1.49 (m, 1H), 1.25 (t, J=7.2 Hz), 1.12 (m, 1H).

### Preparation of Compound 10a

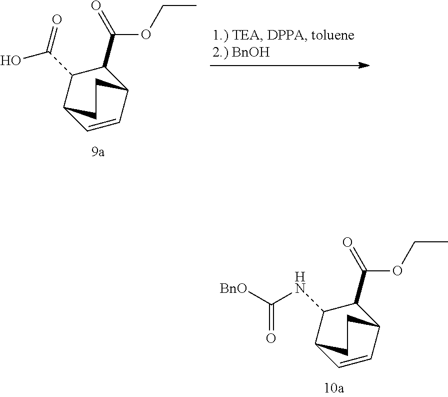 [SMILES: CCOC(=O)[C@@H]1[C@@H](C(=O)O)[C@H]2C=C[C@@H]1CC2 ; CCOC(=O)[C@@H]1[C@@H](NC(=O)OCc2ccccc2)[C@H]2C=C[C@@H]1CC2]

A three neck flask equipped with a mechanical stirrer, temperature probe, reflux condenser, addition funnel and nitrogen inlet was charged with Compound 9a (145.0 g, 1 equiv.) and anhydrous toluene (Aldrich, cat #244511) (1408 g, 1655 mL) under an atmosphere of nitrogen. Then triethylamine (Aldrich, cat #471283) (140 g, 193 mL, 2.14 equiv.) was added in portions over 5 minutes to the stirred solution during which an exotherm to a maximum temperature of 27° C. was observed. Data acquisition by ReactIR was started. The reaction mixture was then heated to 95° C. over 70 minutes. Then diphenyl phosphoryl azide (Aldrich, cat #178756) (176.2 g; 138.0 mL, 0.99 equiv.) was added by addition funnel in portions over a total time of 2.25 hours.

Following completion of the addition of diphenyl phosphoryl azide (addition funnel rinsed with a small amount of toluene), the resulting mixture was heated at 96° C. for an additional 50 minutes. A sample of the reaction mixture diluted in toluene was analyzed by GC/MS which indicated consumption of diphenyl phosphoryl azide. Then benzyl alcohol (Aldrich, cat #108006) (69.9 g, 67.0 mL, 1.0 equiv.) was added by addition funnel over 5-10 minutes. The resulting mixture was then heated at 97° C. overnight (for approximately 19 hours). A sample of the reaction mixture diluted in toluene by GC/MS indicated formation of product (m/e=330). The reaction mixture was then cooled to 21° C. after which water (870 g, 870 mL) was added in portions (observed slight exotherm to maximum temperature of 22° C.). The reaction mixture was first quenched by addition of 500 g of water and mechanically stirred for 10 minutes. The mixture was then transferred to the separatory funnel containing the remaining 370 g of water and then manually agitated. After agitation and phase separation, the organic and aqueous layers were separated (aqueous cut at pH of ˜10). The organic layer was then washed with an additional portion of water (870 g; 1×870 mL). The organic and aqueous layers were separated (aqueous cut at pH of ˜10). The collected organic phase was then concentrated to dryness under reduced pressure (water bath at 45-50° C.) affording 215 g of crude Compound 10a (approximate volume of 190 mL). The ¹H NMR and GC/MS conformed to compound 10a (with residual toluene and benzyl alcohol).

### Preparation of Compound Ha

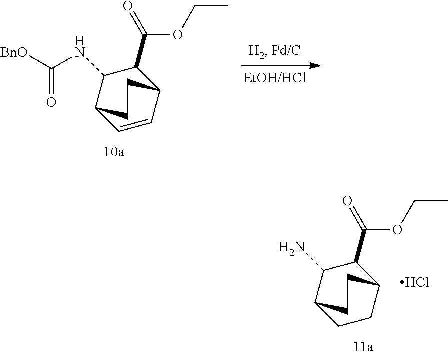 [SMILES: CCOC(=O)[C@@H]1[C@@H](NC(=O)OCc2ccccc2)[C@H]2C=C[C@@H]1CC2 ; CCOC(=O)[C@H]1[C@H]2CC[C@H](CC2)[C@@H]1N ; Cl]

HCl in Ethanol Preparation:

A three neck flask equipped with a temperature probe, nitrogen inlet and magnetic stirrer was charged with ethanol (1000 mL, 773 g) under a nitrogen atmosphere. The solution was stirred and cooled in a dry ice/acetone bath until an internal temperature of −12° C. was reached. Then anhydrous HCl (˜80 g, 2.19 moles) was slowly bubbled in the cooled solution (observed temperature of ˜24 to −6° C. during addition) over 2 hours. Following the addition, the solution was transferred to a glass bottle and allowed to warm to ambient temperature. A sample of the solution was submitted for titration giving a concentration of 2.6 M. The solution was then stored in the cold room (approximately 5° C.) overnight.

Hydrogenation HCl Salt Formation:

A glass insert to a 2 gallon Parr autoclave was charged with palladium on carbon (Pd/C (Aldrich, cat #330108), 10% dry basis; (50% wet), 13.11 g, 0.01 equiv. on the basis of Compound 10a) under a nitrogen atmosphere and then moistened with ethanol (93 g; 120 mL). Then a solution of crude Compound 10a (212 g, 1 eq.) in ethanol (1246 g; 1600 mL) was added to the glass insert (small rinse with ethanol to aid with transfer). The glass insert was placed in the autoclave after which HCl in ethanol (prepared as described above; 2.6 M; 1.04 equiv. based on Compound 10a; 223 g; 259 mL) was added. The autoclave was sealed and then purged with hydrogen (3× at 20 psi). The hydrogenation was then started under an applied pressure of hydrogen gas (15 psi) for 3 hours at which time the pressure of hydrogen appeared constant. Analysis of an aliquot of the reaction mixture by ¹H NMR and GC/MS indicated consumption of starting material/formation of product. The resulting mixture was then filtered over a bed of Celite (192 g) after which the Celite bed was washed with additional ethanol (3×; a total of 1176 g of ethanol was used during the washes). The filtrate (green in color) was then concentrated under reduced pressure (water bath at 45° C.) to 382 g ((˜435 mL; 2.9 volumes based on theoretical yield of Compound 11a. Then isopropyl acetate (1539 g; 1813 mL (12 volumes based on theoretical yield of Compound 11a was added to the remainder. The resulting solution was distilled under vacuum with gradual increase in temperature.

The distillation was stopped after which the remaining solution (370 g, −365 mL total volume; brownish in color) was allowed to stand at ambient temperature over the weekend. The mixture was filtered (isopropyl acetate used to aid with filtration) and the collected solids were washed with additional isopropyl acetate (2×116 mL; each wash was approximately 100 g). The solid was then dried under vacuum at 40° C. (maximum observed temperature of 42° C.) overnight to afford 118 g (78.1% over two steps) of Compound 11a. The ¹H NMR of the material conformed to the structure of Compound 11a, and GC/MS indicated 99% purity.

### Preparation of Compound 13a

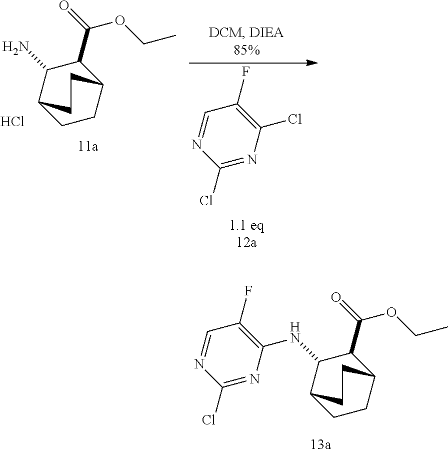 [SMILES: Cl ; CCOC(=O)[C@H]1[C@H]2CC[C@H](CC2)[C@@H]1N ; Fc1cnc(Cl)nc1Cl ; CCOC(=O)[C@H]1[C@H]2CC[C@H](CC2)[C@@H]1Nc1nc(Cl)ncc1F]

Procedure A:

A mixture of 5-fluoro-2,4-dichloropyrimidine (12a, 39.3 g, 235 mmol, 1.1 equiv.), and HCl amine salt (11a, 50 g, 214 mmol) was treated with CH₂Cl₂ (169 mL) and the mixture was warmed to 30° C. The mixture was then treated slowly with DIEA (60.8 g, 82 mL, 471 mmol, 2.2 equiv.) via syringe pump over 3 h. Peak temp was up to 32° C. The reaction was stirred for 20 h, the reaction mixture was judged complete by HPLC and cooled to room temperature. The resulting reaction mixture was washed sequentially with water (211 mL, pH=8-9), 5% NaHSO₄ (211 mL, pH=1-2) then 5% aq. NaCl (211 mL, pH=5-6).

The organic phase was then distilled under reduced pressure to 190 mL. PhMe was charged (422 mL) and temperature set at 70-80° C. and internal temp at 60-65° C. until vol. back down to 190 mL. The mixture was allowed to cool to approximately 37° C. with stirring—after approximately 10 min, crystallization began to occur and the temperature was observed to increase to approximately 41° C. After equilibrating at 37° C., the suspension was charged with n-heptane (421 mL) over 3.5 h followed by cooling to 22° C. over 1 h. The mixture was allowed to stir overnight at that temperature before filtering. The resulting solid on the filter was washed with a 10% PhMe in n-heptane solution (2×210 mL). The solid was then dried in the oven under vacuum with an N₂ purge at 50° C. overnight. The resulting solid weighed 62 g (88% yield).

Procedure B:

A three neck flask equipped with a mechanical stirrer, temperature probe, reflux condenser, nitrogen inlet and addition funnel was charged with Compound 11a (51.2 g) and Compound 12a (40.2 g) under an atmosphere of nitrogen. Dichloromethane (173 mL, 230 g) was added and the resulting mixture was stirred while warming to an internal temperature of 30° C. Then, N,N-diisopropylethylamine (85 mL, 63.09 g) was slowly added by addition funnel over 2.5-3 hours during which time an exotherm to a maximum observed temperature of 33.5° C. was observed. After complete addition, the resulting solution was stirred at 30-31° C. overnight under a nitrogen atmosphere (for approximately 19 hours).

A 100 μL sample of the reaction mixture was diluted with dichloromethane up to a total volume of 10 ml and the solution mixed well. A sample of the diluted aliquot was analyzed by GC/MS which indicated the reaction to be complete by GC/MS; observed formation of product (m/e=328)). The reaction mixture was cooled to 26° C. and transferred to a separatory funnel (aided with dichloromethane). The mixture was then sequentially washed with water (211 mL, 211 g; pH of aqueous cut was ˜8; small rag layer was transferred with aqueous cut), 5% aqueous NaHSO₄ ((prepared using 50 g of sodium bisulfate monohydrate (Aldrich cat. #233714) and 950 g water) 211 mL, 216 g; pH of aqueous cut was ˜2) and then 5% aqueous NaCl ((prepared using 50 g of sodium chloride (Aldrich cat. #S9888) and 950 g water) 211 mL, 215 g; pH of aqueous cut was ˜4-5). The collected organic phase was then concentrated under reduced pressure (water bath at 35° C.) to ˜190 mL (2.7 volumes based on theoretical yield of Compound 13a after which toluene (Aldrich cat. #179418, 422 mL, 361 g) was added. The resulting mixture was concentrated under reduced pressure (water bath at 55-65° C.) to ˜190 mL (2.7 volumes based on theoretical yield of Compound 13a. Analysis of a sample of the solution at this stage by ¹H NMR indicated the absence of dichloromethane. The remaining mixture was allowed to cool to 37° C. (using water bath at 37° C. on rotovap with agitation). During this time pronounced crystallization was observed. The mixture was then mechanically stirred and heated to approximately 37° C. (external heat source set to 38° C.) after which n-heptane (430 mL, 288 g; Aldrich cat. #H2198) was slowly added by addition funnel over 3 hours. Following the addition, heating was stopped and the resulting slurry mechanically stirred while cooling to ambient temperature overnight. The resulting mixture was then filtered and the collected solids were washed with 10% toluene in n-heptane (2×210 mL; each wash was prepared by mixing 21 mL (16 g) of toluene and 189 mL (132 g) of n-heptane). Vacuum was applied until very little filtrate was observed. The solids were then further dried under vacuum at 50° C. under a nitrogen bleed to constant weight (3.5 hours) giving 64.7 g (90%) of Compound 13a. Analysis of a sample of the solid by ¹H NMR showed the material to conform to structure and LC analysis indicated 99.8% purity using the supplied LC method.

### Preparation of Compound 14a

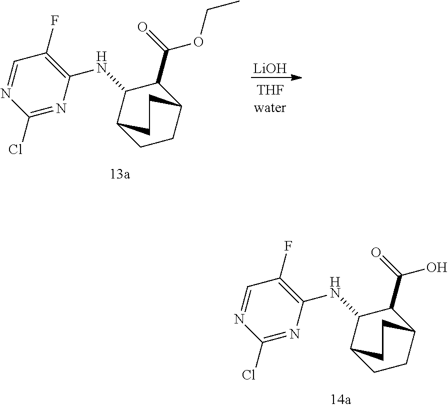 [SMILES: CCOC(=O)[C@H]1[C@H]2CC[C@H](CC2)[C@@H]1Nc1nc(Cl)ncc1F ; "LiOH" ; O=C(O)[C@H]1[C@H]2CC[C@H](CC2)[C@@H]1Nc1nc(Cl)ncc1F]

The ethyl ester 13a (85 g, 259 mmol) was dissolved in THF (340 mL) and treated with a solution of LiOH (2M, 389 mL, 778 mmol) over 10 min (temp from 21 to 24° C.). The mixture was warmed to 45° C. with stirring for 17 h at which time the reaction was judged complete by HPLC (no starting material observed). The reaction mixture was cooled to rt and CH₂Cl₂ was added (425 mL). A solution of citric acid (2 M, 400 mL) was then added slowly over 45 min (temp up to 26° C.). It was noted that during the charge some white solids were formed but quickly dissolved with stirring. The reaction mixture was stirred for an additional 15 min before phases were allowed to separate. After the phases were split, the aqueous phase pH was measured pH=4.0. The organic phase was washed (15 min stir) with water (255 mL)—phases were allowed to separate. The lower layer (organic) containing the desired product was then stored in the fridge overnight.

The organic phase was concentrated under reduced pressure (pot set to 65° C.) to 150 mL (est. 1.76 vol. with respect to starting material). IPA (510 mL) was charged and distilled under reduced pressure (85° C. chiller temp setting) to 255 mL (3 vol.). The level of solvent was brought to approximately 553 mL (6.5 vol.) by the addition of IPA (298 mL). Water (16 mL) was then added and the reaction mixture warmed to reflux (77° C.) with good agitation which dissolved solids precipitated on the walls of the vessel. Reaction mixture was then cooled slowly to 65° C. (over 60 min) and held there—all material still in solution (sample pulled for residual solvent analysis). The reaction was further cooled to 60° C. and the reaction mixture appeared slightly opaque. After stirring for 15 min further cooled to 55° C. While more product precipitates, the mixture is still thin and easily stirred. Water (808 mL) was added very slowly (2.5-3 hrs) while maintaining the temperature around 55 C. The mixture was then cooled to 22° C. over 2 h and allowed to stir overnight. Material was then filtered and washed with a mixture of water:IPA (75:25, 2×255 mL). The acid was dried in a vac oven at 55° C. overnight. Obtained 69 g of acid 14a, 88% yield of a white solid. The material analyzed >99% purity by HPLC.

### Preparation of Compound 15a: Suzuki Coupling

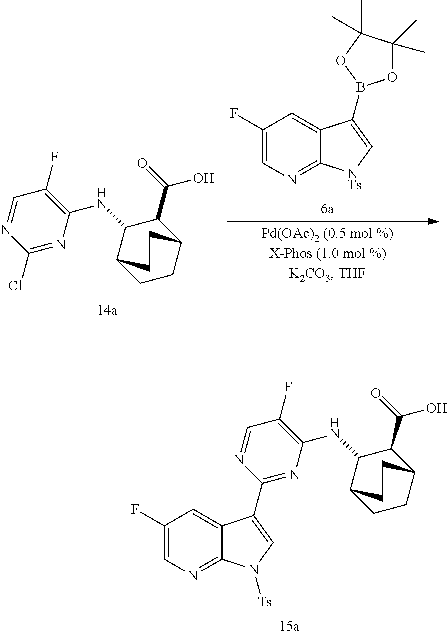 [SMILES: O=C(O)[C@H]1[C@H]2CC[C@H](CC2)[C@@H]1Nc1nc(Cl)ncc1F ; Cc1ccc(S(=O)(=O)n2cc(B3OC(C)(C)C(C)(C)O3)c3cc(F)cnc32)cc1 ; Cc1ccc(S(=O)(=O)n2cc(-c3ncc(F)c(N[C@H]4[C@H]5CC[C@H](CC5)[C@@H]4C(=O)O)n3)c3cc(F)cnc32)cc1]

To 14a (91.4 g, 305 mmol), 6a (158.6 g, 381 mmol, 1.25 equiv.), Pd(OAc)₂ (0.34 g, 1.5 mmol, 0.5 mol %), X-Phos (1.45 g, 3.0 mmol, 1.0 mol %), and K₂CO₃ (168.6 g, 1220 mmol, 4 equiv.) was added THF (731 mL, 8 volumes) and water (29 mL, 0.32 vol.). The reaction mixture was sparged with N₂ for 30 min, then warmed to 65-70° C. and stirred for 5 h. HPLC analysis of the reaction mixture showed 99.3% conversion. The reaction mixture was cooled to 22-25° C. and water was added. The mixture was stirred, the phases were allowed to separate, and the aqueous phase was decanted. A solution of 18 wt % NaCl in water (half-saturated aqueous NaCl) was added to the organic phase and the pH of the mixture was adjusted to 6.0-6.5 using 2N HCl. The phases were allowed to separate and the aqueous phase was decanted. The organic phase was concentrated to a minimum volume and acetonitrile was added. The process was repeated one more time and acetonitrile was added to bring the final volume to 910 mL (10 vol.). The slurry was warmed to 80-85° C. for 6 h, then cooled to 20-25° C. The slurry was stirred for 2 h, then filtered. The solids were rinsed with acetonitrile to give 15a (161 g, 89% yield).

### Preparation of Compound (1): Detosylation Step

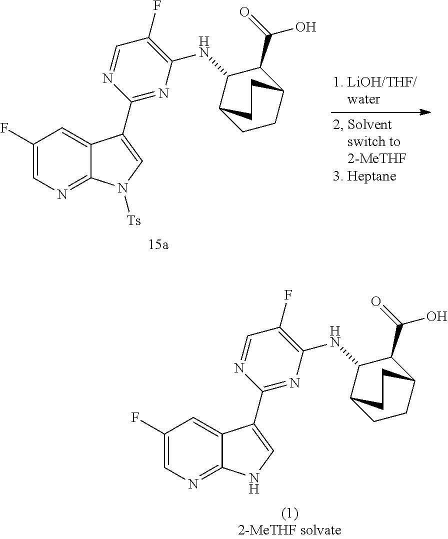 [SMILES: Cc1ccc(S(=O)(=O)n2cc(-c3ncc(F)c(N[C@H]4[C@H]5CC[C@H](CC5)[C@@H]4C(=O)O)n3)c3cc(F)cnc32)cc1 ; O=C(O)[C@H]1[C@H]2CC[C@H](CC2)[C@@H]1Nc1nc(-c2c[nH]c3ncc(F)cc23)ncc1F]

To 15a (25 g, 45.2 mmol) was added THF (125 mL, 5 vol.), then MP-TMT resin (6.25 g, 25 wt %). The mixture was stirred at 20-25° C. for 16 h and filtered, rinsing with 1 vol. THF. The resin treatment process and filtration were repeated. The THF solution was concentrated to 5 vol. To the mixture at 22-25° C. was added an aqueous solution of 2M LiOH (90.3 mL, 4 equiv.). The reaction mixture was warmed to 40-45° C. and stirred for 5 h. HPLC analysis showed 99.7% conversion. The reaction mixture was cooled to 22-25° C. and MTBE (50 mL, 2 vol.) was added. Phase separation occurred. The lower aqueous phase was collected. The aqueous phase was extracted with MTBE. The lower aqueous phase was collected. To the aqueous phase was added 2-MeTHF and the mixture was stirred. The pH of the mixture was adjusted to 6.0-6.5, and the lower aq. phase was decanted. The organic phase was washed with pH 6.5 buffer. The organic phase was concentrated to 85 mL, diluted with 2-MeTHF (150 mL), and concentrated to a final volume of 180 mL. The resultant slurry was warmed to 70-75° C. and stirred until complete dissolution, then cooled to 45-50° C. to give slurry. The slurry was stirred for 1 h, then heptane (180 mL) was added. The slurry was cooled to 20-25° C. over 1 h and stirred for 16 h. The batch was filtered, rinsing the solids with heptane. The solids were dried to give crude Compound (1).1(2-MeTHF) solvate, 79% yield.

The peaks from the XRPD pattern of Compound (1).1(2-MeTHF) are summarized in Table 1 below.

TABLE 1
XRPD peaks for Compound (1)•1(2-MeTHF).

| XRPD Peaks | Angle (2-Theta ± 0.2) | Intensity % |
|---|---|---|
| 1 | 6.4 | 9.78 |
| 2 | 8.4 | 38.07 |
| 3 | 9.7 | 43.96 |
| 4 | 12.9 | 15.57 |
| 5 | 16.7 | 100 |
| 6 | 16.9 | 46.55 |
| 7 | 17.4 | 18.67 |
| 8 | 19.4 | 16.54 |
| 9 | 20.0 | 14.62 |
| 10 | 21.0 | 20.4 |
| 11 | 21.3 | 13.58 |
| 12 | 22.3 | 37.59 |
| 13 | 24.3 | 15.36 |
| 14 | 25.7 | 16.34 |
| 15 | 25.9 | 10.06 |

### Example 2: Preparation of Compound (1) HCl Hemihydrate Salt Form A

Form A of the HCl salt of Compound (1).½H₂O was prepared by mixing Compound (1).1(2-MeTHF) with hydrogen chloride in a mixture of water and an organic solvent(s), wherein the mixture of water and an organic solvent(s) had a water activity of 0.05-0.85. Particular reaction conditions employed are summarized in Table 2 below.

TABLE 2
Reaction Conditions Employed for the Preparation
of Compound (1) HCl hemihydrate salt Form A.

| Comp. (1) | | | | 6N aqueous | | Eq | |
|---|---|---|---|---|---|---|---|
| (mg) 1 (2- | | Solvent | Water | HCl | T | (HCl:Compound | Water |
| MeTHF) | Solvent | (mL) | (mL) | (mL) | (° C.) | (1)) | (wt %) |
| 40 | Acetone | 640 | 40 | 15.70 | 35 | 1.1332 | 8.84% |
| 25 | Acetone | 400 | 25 | 9.80 | 46 | 1.1318 | 8.84% |
| 10.09 | Acetone | 160 | 64 | 3.98 | 35 | 1.1389 | 32.71% |
| 5 | n-propanol | 186 | 10 | 1.29 | 20 | 0.7449 | 6.87% |
| 6.01 | iso-propanol | 88 | 2 | 2.31 | 35 | 1.1097 | 5.10% |
| 6.6 | iPrOH/Acetic | 100/1.0 | 4 | 3.10 | 45 | 1.3561 | 7.25% |
| | Acid => Acetone* | | | | | | |
| 18 | Acetone | 180 | 6 | 3.60 | 30 | 0.5774 | 5.33% |
| 18 | Acetone | 180 | 8 | 6.40 | 35 | 1.0266 | 7.73% |
| 6 | Acetone | 66 | 11 | 2.82 | 30 | 1.3561 | 18.57% |
| 0.101 | iBuOAc | 5 | 0.1 | 0.10 | ~20 | 2.8586 | 4.36% |
| 6 | Acetic Acid | 50 | 8.7 | 2.18 | 35 | 1.0499 | 15.37% |

*two steps: iPrOH/AcOH and then re-slurry in acetone/water

Alternatively, Compound (1) HCl hemihydrate salt Form A was also prepared by the following procedures:

Procedure A:

Compound (1).1(2-MeTHF) (953 g, 2.39 mol) was placed in a 30 L jacketed reactor and treated with IPA (15 L) and water (0.57 L). The stirrer was started and the reaction mixture was warmed to 73° C. to get everything into solution then cooled to 50-55° C. At 50-55° C. the reaction mixture was treated with freshly prepared HCl in IPA (0.83 M, 4.34 L) via slow addition over 4 h. The reaction was sampled, to check for the correct form by XRPD. After the addition, the chiller was programmed to ramp to 0° C. over 480 min with stirring. After form confirmation by XRPD analysis, the slurry was filtered into two filters. The reactor was washed with 3 L of IPA and each filter cake was washed with ˜1.5 L of the rinse IPA from the reactor. The cakes were allowed to air dry with suction overnight. The cakes were then placed in a tray dryer with no heating under vacuum with N₂ purge (22″ Hg) for 24 h. Residual solvent and water analysis showed 505 ppm IPA, 8 ppm 2-Me-TH and approximately 2.15% H₂O. The material was pulled from the oven and co-milled to de-lump to provide 805 g of Compound (1) HCl hemihydrate salt Form A.

Procedure B:

Alternatively, acetone was used instead of IPA, but in a similar manner as described above in Procedure A to form Compound (1) HCl hemihydrate salt Form A.

The XRPD and ¹³C SSNMR data of Form A of Compound (1) HCl hemihydrate salt Form A are shown in FIGS. 1 and 2, respectively. Certain observed XRPD peaks and ¹³C SSNMR peaks are summarized in Tables 3 and 4, respectively.

TABLE 3
XRPD Peaks of Form A of Compound
(1) HCl hemihydrate salt Form A.

| XRPD Peaks | Angle (2-Theta ± 0.2) | Intensity % |
|---|---|---|
| 1 | 10.5 | 100.0 |
| 2 | 5.2 | 71.6 |
| 3 | 7.4 | 46.8 |
| 4 | 18.9 | 42.0 |
| 5 | 25.2 | 41.7 |
| 6 | 16.5 | 39.5 |
| 7 | 18.1 | 28.1 |
| 8 | 23.0 | 27.5 |
| 9 | 24.1 | 25.3 |
| 10 | 20.2 | 21.6 |
| 11 | 26.4 | 21.3 |
| 12 | 15.8 | 19.8 |
| 13 | 21.8 | 18.3 |
| 14 | 13.8 | 17.6 |
| 15 | 27.4 | 17.3 |
| 16 | 29.0 | 16.7 |
| 17 | 14.8 | 15.0 |
| 18 | 32.0 | 15.0 |
| 19 | 25.7 | 13.8 |
| 20 | 28.6 | 13.4 |
| 21 | 33.8 | 13.0 |
| 22 | 12.8 | 12.0 |
| 23 | 30.8 | 11.7 |
| 24 | 32.4 | 11.6 |
| 25 | 24.5 | 11.5 |
| 26 | 23.4 | 11.1 |
| 27 | 21.0 | 10.4 |

TABLE 4
¹³C SSNMR Peaks of Compound (1)
HCl hemihydrate salt Form A.

| Peak | Chem Shift | Intensity |
|---|---|---|
| # | [±3 ppm] | [rel] |
| 1 | 180.1 | 50.4 |
| 2 | 157.9 | 9.1 |
| 3 | 154.6 | 26.4 |
| 4 | 150.7 | 25.3 |
| 5 | 144.9 | 31.0 |
| 6 | 140.1 | 6.7 |
| 7 | 132.4 | 36.3 |
| 8 | 131.2 | 30.0 |
| 9 | 129.0 | 21.0 |
| 10 | 117.5 | 33.6 |
| 11 | 114.0 | 38.0 |
| 12 | 107.0 | 34.4 |
| 13 | 54.8 | 42.0 |
| 14 | 47.7 | 52.7 |
| 15 | 29.2 | 100.0 |
| 16 | 24.6 | 74.0 |
| 17 | 22.1 | 83.6 |

The prepared Compound (1) HCl hemihydrate salt Form A was found to be stable in the following solvent systems (but not limited to): chlorobenzene, cyclohexane, 1,2-dichloroethane, dichloromethane, 1,2-dimethoxyethane, hexane, 2-methoxyethanol, methylbutyl ketone, methylcyclohexane, nitromethane, tetralin, xylene, toluene, 1,1,2-trichloroethane, acetone, anisole, 1-butanol, 2-butanol, butyl acetate, t-butylmethylether, cumene, ethanol, ethyl acetate, ethyl ether, ethyl formate, heptane, isobutyl acetate, isopropyl acetate, methyl acetate, 3-methyl-1-butanol, methylethyl ketone, 2-methy-1-propanol, pentane, 1-propanol, 1-pentanol, 2-propanol, propyl acetate, tetrahydrofuran, and methyl tetrahydrofuran.

Specifically, for the solubility and stability tests for Compound (1) HCl hemihydrate salt Form A, samples of the compound were loaded into 2 mL HPLC vials with 500 μL of solvent. The mixture was stirred at ambient temperature for 2 weeks and then filtered by centrifuge. The resulting solids were analyzed by XRPD, solutions were analyzed for solubility by quantitative NMR against hydroquinone standard. The results are summarized in Table 5.

TABLE 5
Summary of form and solubility data for Compound
(1) HCl hemihydrate salt Form A (A = Compound
(1) HCl hemihydrate salt Form A; D = different
polymorphic form, e.g. a form incorporating the solvent).

| Solvent | Solubility (mg/mL) | Resulting Form |
|---|---|---|
| Acetonitrile | 0.5 | D |
| Chlorobenzene | <0.1 | A |
| Chloroform | <0.1 | D |
| Cyclohexane | <0.1 | A |
| 1,2-Dichloroethane | 1.7 | A |
| Dichloromethane | 0.1 | A |
| 1,2-Dimethoxyethane | 0.5 | A |
| 1,4-Dioxane | 0.4 | A |
| Ethylene glycol | 108.1 | D |
| Hexane | <0.1 | A |
| Methanol | 46.4 | D |
| 2-Methoxyethanol | 34.1 | A |
| Methylbutyl ketone | 0.4 | A |
| Methylcyclohexane | <0.1 | A |
| Nitromethane | <0.1 | A |
| Tetralin | <0.1 | A |
| Toluene | <0.1 | A |
| 1,1,2-Trichloroethane | <0.1 | A |
| Xylene | <0.1 | A |
| Acetone | 1.5 | A |
| Anisole | <0.1 | A |
| 1-Butanol | 2.9 | A |
| 2-Butanol | 2.9 | A |
| Butyl acetate | 0.2 | A |
| t-Butylmethylether | 0.4 | A |
| Cumene | <0.1 | A |
| Dimethylsulfoxide | 346.5 | D |
| Ethanol | 19.9 | A |
| Ethyl acetate | 0.2 | A |
| Ethyl ether | 0.1 | A |
| Ethyl formate | 0.4 | A |
| Formic acid | 214.0 | D |
| Heptane | <0.1 | A |
| Isobutyl acetate | 0.2 | A |
| Isopropyl acetate | 0.4 | A |
| Methyl acetate | 0.6 | A |
| 3-Methyl-1-butanol | 3.2 | A |
| Methylethyl ketone | 0.5 | A |
| 2-Methy-1-propanol | 3.5 | A |
| Pentane | <0.1 | A |
| 1-Pentanol | 3.3 | A |
| 1-Propanol | 10.7 | A |
| 2-Propanol | 3.3 | A |
| Propyl acetate | 0.8 | A |
| Tetrahydrofuran | 0.7 | A |
| Methyl tetrahydrofuran | 0.7 | A |
| Water | 0.6 | D |

Thermogram data was obtained (the data not shown) for Compound (1) HCl hemihydrate salt Form A by placing the sample in a platinum sample pan and by heating at 10° C./min to 300° C. from room temperature. The thermogram data demonstrated a weight loss of 2.1% from 30° to 170° C. which was consistent with theoretical hemihydrate (2.0%).

DSC thermogram data was obtained (the data not shown) for Compound (1) HCl hemihydrate salt Form A by heating the sample at 10° C./min to 300° C. from room temperature. DSC thermogram showed a dehydration onset temperature of 50-100° C. followed by an onset melting/decomposition temperature of 200-260° C.

### Example 3: Process for Determining Tablet Dissolution

There are four specific quality control (QC) dissolution methods (QC-1, 2, 3, and 4) utilized in the present invention, as well as one physiology-based (PB) method that utilizes a simulated intestinal fluid as the medium. The details of these methods are provided in Tables 6 and 7 below. The QC-2 dissolution method is a method for release and stability testing in phase I-phase II clinical trials, and for concept screening. The QC-2 dissolution method was consistent with the dissolution testing method originally used to test the Phase 2b formulation; except, the QC-2 dissolution method employed a paddle speed of 75 rpm while the dissolution method for the Phase 2b formulation employed a paddle speed of 50 rpm.

The QC-3 dissolution method is a method for development and screening purposes, and more closely approximates a biorelevant testing method. The QC-4 dissolution method utilizes the cationic detergent CTAB (cetyl trimethylammonium bromide) in the dissolution medium.

TABLE 6
QC-2, QC-3, and QC-4 dissolution methods.

| | QC-2 | | | QC-4 |
|---|---|---|---|---|
| | method | QC-3 method | | method |
| Apparatus | Paddle | Paddle | | USP Type 2 |
| | (75 rpm) | (50 rpm) | | (75 RPM) |
| | | Phase 1 | Phase 2 | |
| Volume | 900 mL | 300 mL | 900 mL | 900 mL |
| Medium | 150 mM Na | 0.01M HCl | 100 mM Na | 1.0% w/v CTAB |
| | phosphate | pH 2 | phosphate | with 0.1M HCl |
| | buffer pH | | buffer pH | (37.0 ± 0.5° C.) |
| | 7.4 | | 7.4 | |

TABLE 7
QC method vs. PB method to determine dissolution.

| Parameter | QC-1 method | PB method |
|---|---|---|
| Dissolution apparatus | Paddle (USP type 2, Ph. Eur., JP) | |
| Dissolution medium | 37.0 ± 0.5° C. | |
| temp. | | |
| Dissolution medium | 900 mL | |
| volume | | |
| Dissolution medium | 150 mM | Phase 1 (15 minutes): |
| | Sodium | 300 mL of SGFsp* pH 3.0 |
| | Phosphate | Phase 2 (2 hours): |
| | Buffer, pH 7.4 | 900 mL of FaSSIF pH 6.7, |
| | | 600 mL of FaSSIF** |
| | | (preheated at 37° C.) |
| | | was added to the 300 mL |
| | | Phase 1 soltn. |
| | | .The total volume of |
| | | FaSSIF pH 6.7 is 900 mL |
| Paddle rotation speed | 50 rpm | 75 rpm |
| Sample filter | Vankel 10-μm | Whatman Spartan 0.2 μm RC |
| | pore size full | (regenerated cellulose) |
| | flow filter tips | membrane 30-mm diameter |
| | | filter, or equivalent. |

| | |
|---|---|
| Analytical finish | HPLC with UV detection at 274 nm |

*Simulated Gastric Fluid sine pepsine
**Fasting State Simulated Intestinal Fluid

### Example 4: Process for Producing Tablet Compositions 1-6, Having 600 mg Compound (1) (Molar Equivalent of the HCl Salt)

Step 1: Fluid Bed Granulation Process

Binder Solution:

Hydroxypropyl methylcellulose (TPMC) 2910 15 mPa·s (21.00 mg per unit) and polysorbate 20 (3.00 mg per unit; common commercial brand names include Scattics, Alkest TW 20, and Tween 20) were added to purified water (700.00 mg per unit) and mixed until a clear solution was obtained.

Granulation:

Compound (1) HCl hemihydrate salt Form A (668.40 mg per unit) and crospovidone (20.00 mg per unit) were transferred to a fluid bed granulator, and the resulting mixture was warmed while fluidizing. The binder solution was then sprayed upon the ingredients using standard wet granulation techniques.

The granulate was dried while fluidizing, the dried granules collected, and then packed in aluminum bags for later use.

Step 2: Blending and Tableting

Granules comprising Compound (1) HCl hemihydrate salt Form A from step 1 and colloidal anhydrous silica are passed through a sieve (0.950 mm sieve size; 0.4 mm wire diameter) and mixed until homogeneous using a high speed blender (10 rpm, 5 min).

To this mixture was added a second mixture of silicified microcrystalline cellulose, microcrystalline cellulose (if present), pregelatinized starch (if present), and crospovidone that was previously also passed through a sieve (0.950 mm sieve size; 0.4 mm wire) diameter. The resulting mixture was then also blended until homogenous (10 rpm, 10 min).

To this mixture was added sodium stearyl fumarate that was previously also passed through a sieve (0.950 mm sieve size; 0.4 mm wire diameter). The resulting mixture was then also blended until homogenous (10 rpm, 10 min).

The resulting blended mixture was then compressed into tablets using a tableting press. The resulting tablets were then collected into aluminum laminated bags in suitable containers.

Step 3: Film Coating

Coating powder, for example Opadry II White 85F18422, was mixed with purified water (amounts specified in a per unit basis in Tables 8A and 8B below) to create a coating suspension. The core tablets from steps 1 and 2 were transferred to a coating pan and sprayed with the coating suspension using the film coating technique, which comprises 1) loading the tablets into the coating pan and allowing them to pre-warm to the required temperature; 2) spraying the tablets with the coating suspension based on the parameters as set in Table 10 until the required weight film coating layer (weight gain) is achieved on the tablets (“the spraying phase”); and 3) drying the tablets at the set inlet and exhaust air temperature for 5 minutes. The dried, film coated tablets were then transferred to aluminum laminated bags in suitable containers.

The component compositions of Tablet Compositions 1-6 are presented in Tables 8A and 8B below.

TABLE 8A
Tablet Compositions 1-3.

| | Tablet Comp. 1 | | Tablet Comp. 2 | | Tablet Comp. 3 | |
|---|---|---|---|---|---|---|
| Component (Function) | Mg/unit | % w/w | Mg/unit | % w/w | Mg/unit | % w/w |

Core Tablet
Intragranular Phase

| | | | | | | |
|---|---|---|---|---|---|---|
| Compound (1) HCl | 668.40 | 51.42 | 668.40 | 51.42 | 668.40 | 51.42 |
| hemihydrate salt Form A | | | | | | |
| (API) | | | | | | |
| Crospovidone | 20.00 | 1.54 | 20.00 | 1.54 | 20.00 | 1.54 |
| (disintegrant) | | | | | | |
| Purified water (solvent) | 700.00^{a} | | 700.00^{a} | | 700.00^{a} | |
| Hypromellose (HPMC) | 21.00 | 1.62 | 21.00 | 1.62 | 21.00 | 1.62 |
| 2910 15 mPa · s (binder) | | | | | | |
| Tween 20 (wetting agent) | 3.00 | 0.23 | 3.00 | 0.23 | 3.00 | 0.23 |

Extragranular phase

| | | | | | | |
|---|---|---|---|---|---|---|
| SMCC HD90 (diluent) | 334.60 | 25.74 | 334.60 | 25.74 | 334.60 | 25.74 |
| Colloidal anhydrous silica | 13.00 | 1.00 | 13.00 | 1.00 | 13.00 | 1.00 |
| (glidant) | | | | | | |
| Microcrystalline cellulose | — | — | 130.0 | 10.0 | 65.00 | 5.00 |
| (Ceolus) (diluent) | | | | | | |
| Partially pregelatinized | 130.00 | 10.0 | — | — | 65.00 | 5.00 |
| maize starch (Starch | | | | | | |
| 1500) (diluent) | | | | | | |
| Crospovidone | 71.00 | 5.46 | 71.00 | 5.46 | 71.00 | 5.46 |
| (disintegrant) | | | | | | |
| Sodium stearyl fumarate | 39.00 | 3.00 | 39.00 | 3.00 | 39.00 | 3.00 |
| (lubricant) | | | | | | |
| Core tablet weight | 1300.0 | 100.0 | 1300.0 | 100.0 | 1300.0 | 100.0 |

Film coating

| | | | | | | |
|---|---|---|---|---|---|---|
| Opadry II white 85F18422 | 39.00 | +3 | 39.00 | +3 | 39.00 | +3 |
| (film-coat) | | | | | | |
| Purified water (solvent) | 156.0^{a} | | 156.0^{a} | | 156.0^{a} | |
| Total tablet weight | 1339.0 | | 1339.0 | | 1339.0 | |

^{a}Removed during processing

TABLE 8B
Tablet Compositions 4-6.

| | Tablet Comp. 4 | | Tablet Comp. 5 | | Tablet Comp. 6 | |
|---|---|---|---|---|---|---|
| Component (Function) | Mg/unit | % w/w | Mg/unit | % w/w | Mg/unit | % w/w |

Core Tablet
Intragranular Phase

| | | | | | | |
|---|---|---|---|---|---|---|
| Compound (1) HCl | 668.40 | 60.76 | 668.40 | 60.76 | 668.40 | 60.76 |
| hemihydrate salt Form A | | | | | | |
| (API) | | | | | | |
| Crospovidone | 20.00 | 1.82 | 20.00 | 1.82 | 20.00 | 1.82 |
| (disintegrant) | | | | | | |
| Purified water (solvent) | 700.00^{a} | | 700.00^{a} | | 700.00^{a} | |
| Hypromellose (HPMC) | 21.00 | 1.91 | 21.00 | 1.91 | 21.00 | 1.91 |
| 2910 15 mPa · s (binder) | | | | | | |
| Tween 20 (wetting agent) | 3.00 | 0.27 | 3.00 | 0.27 | 3.00 | 0.27 |

Extragranular phase

| | | | | | | |
|---|---|---|---|---|---|---|
| SMCC HD90 (diluent) | 204.10 | 18.55 | 204.10 | 18.55 | 204.10 | 18.55 |
| Colloidal anhydrous silica | 11.00 | 1.00 | 11.00 | 1.00 | 11.00 | 1.00 |
| (glidant) | | | | | | |
| Microcrystalline cellulose | — | — | 82.50 | 7.50 | 41.25 | 3.75 |
| (Ceolus) (diluent) | | | | | | |
| Partially pregelatinized | 82.50 | 7.50 | — | — | 41.25 | 3.75 |
| maize starch (Starch | | | | | | |
| 1500) (diluent) | | | | | | |
| Crospovidone | 57.00 | 5.18 | 57.00 | 5.18 | 57.00 | 5.18 |
| (disintegrant) | | | | | | |
| Sodium stearyl fumarate | 33.00 | 3.00 | 33.00 | 3.00 | 33.00 | 3.00 |
| (lubricant) | | | | | | |
| Core tablet weight | 1100.0 | 100.0 | 1100.0 | 100.0 | 1100.0 | 100.0 |

Film coating

| | | | | | | |
|---|---|---|---|---|---|---|
| Opadry II white 85F18422 | 33.00 | +3 | 33.00 | +3 | 33.00 | +3 |
| (film-coat) | | | | | | |
| Purified water (solvent) | 132.0^{a} | | 132.0^{a} | | 132.0^{a} | |
| Total tablet weight | 1133.0 | | 1133.0 | | 1133.0 | |

^{a}Removed during processing

The parameters and results of the tablet compression process for Compositions 1-6 are provided in Table 9 below.

TABLE 9
Tablet compression parameters for Compositions 1-6.

| Parameter or Result | Comp. 1 | Comp. 2 | Comp. 3 | Comp. 4 | Comp. 5 | Comp. 6 |
|---|---|---|---|---|---|---|
| Bulk density blend | 0.472 | 0.440 | 0.460 | 0.444 | 0.436 | 0.432 |
| (g/mL) | | | | | | |
| Punch size (mm) | 22 × 11 | 22 × 11 | 22 × 11 | 19.7 × 9.5 | 19.7 × 9.5 | 19.7 × 9.5 |
| Compression force | 2300 | 2000 | 2300 | 2200 | 2000 | 2100 |
| (daN) | | | | | | |
| Cracks | No | No | No | No | No | No |
| Filming | No | No | No | No | No | No |
| Compression process | + | + | + | + | + | + |
| (+) means that | | | | | | |
| filming and/or cracks | | | | | | |
| where not observed. | | | | | | |
| The weight variation | | | | | | |
| was acceptable | | | | | | |
| Yield (approximate | 613 | 609 | 613 | 596 | 598 | 576 |
| number of tablets) | | | | | | |
| Disintegration time | 2:42 | 1:53 | 2:15 | 6:26 | 4:24 | 5:28 |
| (minutes: seconds) | (n = 6) | (n = 6) | (n = 6) | (n = 6) | (n = 6) | (n = 6) |
| (av.) | | | | | | |
| Hardness (N) (av.) | 208 | 254 | 240 | 235 | 270 | 255 |
| | (n = 5) | (n = 5) | (n = 5) | (n = 5) | (n = 5) | (n = 5) |
| Thickness (mm) (av.) | 6.62 | 6.60 | 6.57 | 6.13 | 6.13 | 6.15 |
| | (n = 32) | (n = 32) | (n = 32) | (n = 32) | (n = 32) | (n = 32) |
| Weight (mg) (av.) | 1303.5 | 1301.8 | 1302.9 | 1103.6 | 1100.6 | 1104.7 |
| | (n = 32) | (n = 32) | (n = 32) | (n = 32) | (n = 32) | (n = 32) |
| Friability (%) | 0.058 | 0.073 | 0.053 | 0.191 | 0.126 | 0.189 |

The parameters and results of the film coating process are provided in Table 10 below.

TABLE 10
Parameters and results of the film coating process for Compositions 1-6.

| Parameter or Result | Comp. 1 | Comp. 2 | Comp. 3 | Comp. 4 | Comp. 5 | Comp. 6 |
|---|---|---|---|---|---|---|
| Pan coating | | | | | | |
| Amount of coating | 75 | 75 | 75 | 75 | 75 | 75 |
| suspension (g) | | | | | | |
| Spraying | | | | | | |
| Spray time (min) | 23 | 23.5 | 34 | 33 | 31 | 34 |
| Air flow (m³/h) | 90 | 90 | 90 | 90 | 90 | 90 |
| Inlet air temp (° C.) | 76.3-84.5 | 72.7-80.9 | 76.0-80.9 | 84.2-85.7 | 83.4-88.3 | 70.1-86.2 |
| Exhaust temp (° C.) | 42.5-44.0 | 41.3-43.2 | 41.7-44.5 | 43.6-44.7 | 40.1-43.3 | 40.2-45.0 |
| Spray rate (g/min) | 2.6-3.8 | 2.6-3.8 | 2.2-2.6 | 2.4-2.6 | 2.6 | 2.4-2.8 |
| Atomization air (bar) | 1.0-1.2 | 1.2 | 1.2 | 1.2 | 1.2 | 1.2 |
| Pan speed (rpm) | 20 | 20 | 20 | 20 | 20 | 20 |
| Drying | | | | | | |
| Drying time | 5 | 5 | 5 | 5 | 5 | 5 |
| Aesthetic Aspects | White, | White, | White, | White, | White, | White, |
| | smooth | smooth | smooth | smooth | smooth | smooth |
| Film coating layer (%) | 2.39 | 2.57 | 2.36 | 2.16 | 2.25 | 2.38 |
| Film coating process | Yes | Yes | Yes | Yes | Yes | Yes |
| was successful | | | | | | |

Dissolution Tests:

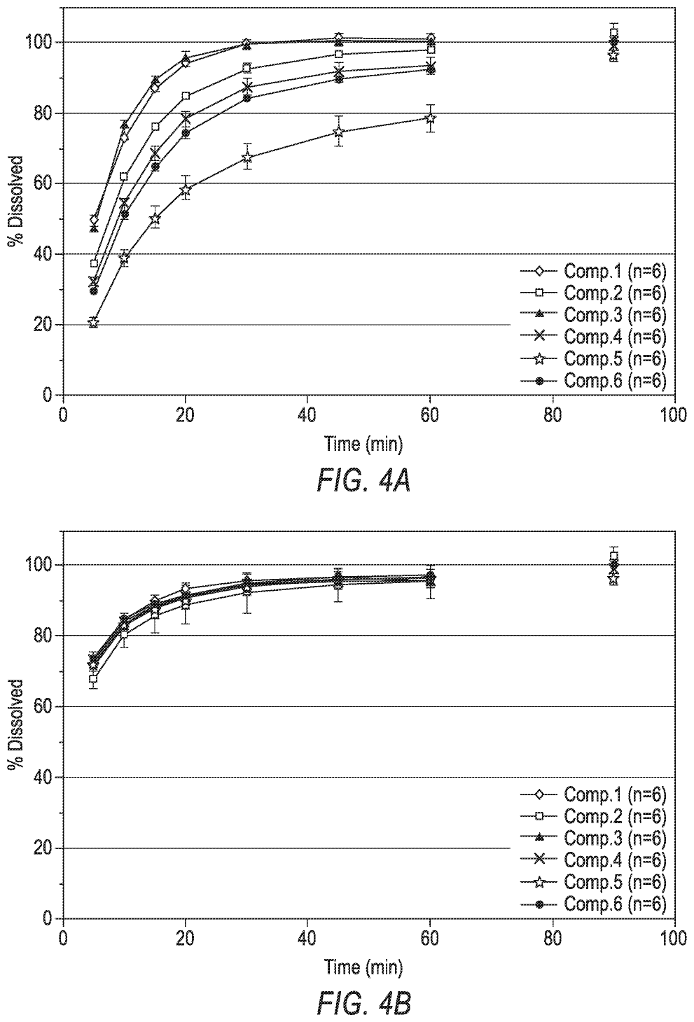

No changes were observed in appearance, assay or degradation of tablet compositions 1-6 at time=0 (T₀) and after stressing for 2 weeks at 50/70% RH open dish conditions. Dissolution profiles for Compositions 1-6 were generated using QC-2 dissolution method and QC-3 dissolution method, as described above and provided in FIGS. 4A and 4B. The data in FIG. 4A demonstrates that Composition 3 possesses superior dissolution properties over Compositions 1, 2, and 4-6.

### Example 5: Process for Producing Tablet Composition 7 Having 300 mg Compound (1) (Molar Equivalent of the HCl Salt Hemihydrate)

Additional process and formula development directed 300 mg tablet compositions by reducing the amounts of excipients from those present in the 600 mg tablet compositions. Composition 8 was developed by dose-proportionally reducing the excipients present in the equivalent 600 mg Composition 3 tablet by 50%. Composition 8 is a formulation that is in between Composition 3 and Composition 7, and serves as a bridging composition between the two.

It was demonstrated that the influence of the API properties was significant in the 300 mg formulations. Low-density APIs caused higher volumetric load in the fluid bed granulator resulting in flow challenges and this yielded granules with a significantly finer particle size, which was associated with flow problems during compression. API lots with higher bulk density and lower Specific Surface Area (SSA) were associated with better granulation outcomes. To overcome these issues, the composition of the intragranular phase was adjusted, increasing the binder and wetting agent concentration to improve binding properties of the granules and wetting of the API during granulation. The compositions of the eq. 300 mg Compound (1) final film coated tablets, is shown in Table 11 below.

TABLE 11
Tablet Composition 7 and 8.

| | Tablet Comp. 7 | | Tablet Comp. 8 | |
|---|---|---|---|---|
| Component (Function) | Mg/unit | % w/w | Mg/unit | % w/w |

Intragranular Phase

| | | | | |
|---|---|---|---|---|
| Compound (1) HCl hemihydrate | 334.20 | 51.42 | 334.20 | 51.42 |
| salt Form A (API) | | | | |
| Crospovidone (disintegrant) | 9.75 | 1.50 | 10.00 | 1.54 |
| Purified water (solvent) | 410.0^{a} | | 350.00^{a} | |
| Hypromellose (HPMC) 2910 15 | 12.35 | 1.90 | 10.50 | 1.62 |
| mPa · s (binder) | | | | |
| Polysorbate 20 (Tween 20) | 3.25 | 0.50 | 1.50 | 0.23 |
| (wetting agent) | | | | |
| Total Intragranular phase | 359.55 | | 356.20 | |

Extragranular phase

| | | | | |
|---|---|---|---|---|
| Silicified microcrystalline | 150.70 | 23.18 | 167.30 | 25.74 |
| cellulose (SMCC HD90) | | | | |
| (diluent) | | | | |
| Colloidal anhydrous silica | 6.50 | 1.00 | 6.50 | 1.00 |
| (glidant) | | | | |
| Microcrystalline cellulose | 32.50 | 5.00 | 32.50 | 5.00 |
| (Ceolus) (diluent) | | | | |
| Partially pregelatinized | 32.50 | 5.00 | 32.50 | 5.00 |
| maize starch (Starch 1500) | | | | |
| (diluent) | | | | |
| Crospovidone (disintegrant) | 35.75 | 5.50 | 35.50 | 5.46 |
| Sodium stearyl fumarate | 32.50 | 5.00 | 19.50 | 3.00 |
| (lubricant) | | | | |
| Total EF | 290.45 | | 293.80 | |
| Total Core Tablet^{b} | 650.00 | 100 | 650.00 | 100 |

Film Coating

| | | | | |
|---|---|---|---|---|
| Opadry II Yellow 85F92450 | 19.50 | +3.00 | 19.50 | +3.00 |
| (Coating powder) | | | | |
| Purified water (solvent) | 78.00^{a} | | 78.00^{a} | |

^{a}Removed during processing.
^{b}Tablets are coated to a target coating weight of 3%.

Parameters and results of the tablet compression process for Tablet Composition 7 are provided in Tables 12 and 13 below. The tables present results for a single batch of Tablet Composition 7 at 1) four different sampling intervals during compression (Table 12) and 2) five different sampling intervals during compression (Table 13).

TABLE 12
Process parameters and IPCs of the compression of Tablet Composition 7.

| | Comp. 7 | | Comp. 7 | | Comp. 7 | | Comp. 7 | |
|---|---|---|---|---|---|---|---|---|
| Parameter or Result | (Sample 1) | | (Sample 2) | | (Sample 3) | | (Sample 4) | |
| Compression speed | 650 | | 648 | | 648 | | N/A | |
| (tpm) | | | | | | | | |
| Ejection force (daN) | 27 | | 25 | | 24 | | N/A | |
| Compression force | 1892 | | 1872 | | 1929 | | N/A | |
| (daN) | | | | | | | | |
| Weight^{b} (mg) (av.) | 656.6 | (1.19) | 653.6 | (1.09) | 650.4 | (1.13) | 652.8 | (0.92) |
| | 643.8-664.6 | | 644.5-662.4 | | 640.8-660.8 | | 643.5-660.1 | |
| Thickness^{b} (mm) (av.) | 5.13 | (0.61) | 5.08 | (0.51) | 5.06 | (0.30) | 5.08 | (0.53) |
| | 5.08-5.16 | | 5.06-5.12 | | 5.04-5.08 | | 5.05-5.12 | |
| Hardness^{b} (N) (av.) | 212 | (3.67) | 208 | (1.26) | 212 | (4.82) | 215 | (2.93) |
| | 204-224 | | 204-210 | | 202-224 | | 206-222 | |
| Disintegration time^{b} | 6:32 | (15.8) | 5:31 | (16.4) | 5:51 | (9.0) | 7:47 | (14.1) |

| | | | | |
|---|---|---|---|---|
| (minutes:seconds) (av.) | 5:09-7:47 | 4:37-7:01 | 5:13-6:39 | 6:00-8:82 |

^{b}These IPCs are reported as mean (RSD) and min-max on the second line.

TABLE 13
Process parameters and IPCs of the compression of Tablet Composition 7.

| Parameter | Comp. 7 | | Comp. 7 | | Comp. 7 | | Comp. 7 | | Comp. 7 | |
|---|---|---|---|---|---|---|---|---|---|---|
| or Result | (Sample 1) | | (Sample 2) | | (Sample 3) | | (Sample 4) | | (Sample 5) | |
| Compression speed | 751 | | NA | | 751 | | NA | | 751 | |
| (tpm) | | | | | | | | | | |
| Ejection force (daN) | 19 | | NA | | 20 | | NA | | 19 | |
| Compression force | 1922 | | NA | | 1976 | | NA | | 1984 | |
| (daN) | | | | | | | | | | |
| Weight^{b} (mg) (av.) | 649.0 | (0.36) | 648.6 | (0.59) | 648.7 | (0.40) | 653.6 | (0.40) | 650.4 | (0.35) |
| | 645.4-651.5 | | 642.9-653.9 | | 643.3-653.1 | | 649.5-656.8 | | 645.8-653.1 | |
| Thickness^{b} (mm) (av.) | 5.06 | (0.26) | 5.06 | (0.82) | 5.05 | (0.22) | 5.05 | (0.30) | 5.05 | (0.22) |
| | 5.05-5.08 | | 5.04-5.08 | | 5.04-5.06 | | 5.03-5.07 | | 5.04-5.06 | |
| Hardness^{b} (N) (av.) | 206 | (1.68) | 208 | (1.78) | 209 | (2.32) | 208 | (2.11) | 205 | (1.52) |
| | 204-212 | | 204-214 | | 205-217 | | 204-215 | | 200-208 | |
| Disintegration time^{b} | 7:10 | (5.9) | 8:43 | (6.6) | 6:55 | (5.2) | 7:25 | (18.9) | 6:55 | (8.9) |

| | | | | | |
|---|---|---|---|---|---|
| (minutes:seconds) (av.) | 6:35-7:51 | 7:55-9:23 | 6:34-7:32 | 5:57-9:54 | 5:54-7:48 |

^{b}These IPCs are reported as mean (RSD) and min-max on the second line.

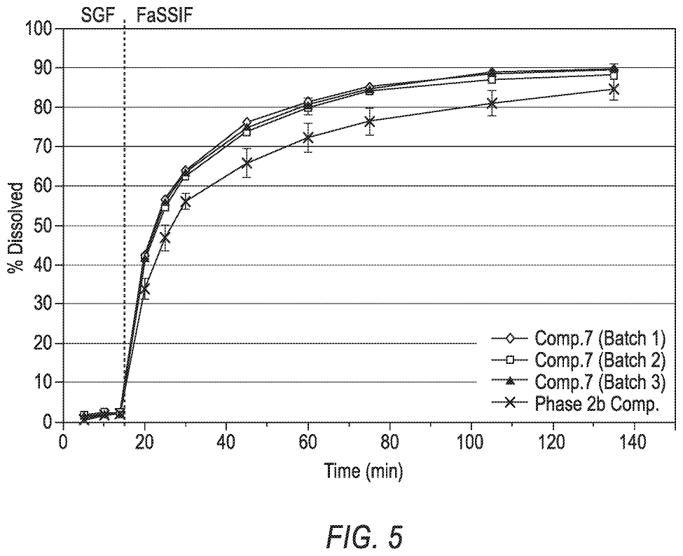

The drug release in vitro was tested using a physiological based dissolution testing method (PBDT) which uses simulated intestinal fluid as the dissolution medium. In vitro dissolution in biorelevant (PBDT) media can be used as a model for the in vivo performance of drugs (i.e. dissolution in the gastrointestinal tract), as the media is designed to mimic the physicochemical conditions of the gastrointestinal tract. Specifically, the physiology based dissolution method (PBDT) was used to compare the dissolution of Tablet Composition 7 against the Phase 2b formulation. As provided by FIG. 5, under the PBDT method, the Phase 2b formulation has a dissolution profile slower than the dissolution profile for Tablet Composition 7. Accordingly, Tablet Composition 7 had a dissolution profile which was superior to the Phase 2b formulation.

The conditions for granulation of Tablet Composition 8 are provided in Table 14 below.

TABLE 14
Granulation conditions for Tablet Composition 8.

| Element | Pre-warming | | Granulation | | Drying | |
|---|---|---|---|---|---|---|
| Spray Nozzle | — | | 1 | mm | — | |
| Air Flow | 600 | m³/h | 800-1200 | m³/h | 1000-1200 | m³/h |
| Spray rate | — | | 250 | g/min | — | |
| Atomizing air flow | 2 | bar | 2 | bar | 2 | bar |
| Inlet air temperature | 60° | C. (set) | 45° | C. (set) | 65-70° | C. (set) |
| Outlet air temperature | 40° | C. (end) | 23° | C. (end) | 43° | C. (end) |
| Product temperature | 40° | C. (end) | 23° | C. (end) | 31° | C. (end) |

| | | | | | |
|---|---|---|---|---|---|
| Spraying/drying time | 5 | min | 1 h 22 min | 33 | min |

| | | | |
|---|---|---|---|
| LOD end of phase | — | 24.19% | 1.06% |

Compression parameters and physical attributes of the tablet cores of Tablet Composition 8 are provided in Table 15 below.

TABLE 15
Compression parameters and physical attributes
of tablet cores for Tablet Composition 8.

| Parameter or Result | Sample 1 | Sample 2 | Sample 3 | Sample 4 |
|---|---|---|---|---|
| Compression speed (tpm) | 750 | 750 | 750 | N/A |
| Ejection force (daN) | 56 | 56 | 52 | N/A |
| Compression force (daN) | 1937 | 1625 | 1578 | N/A |
| Weight^{b} (mg) (av.) | 655.2 | 651.6 | 651.1 | 649.2 |
| Thickness^{b} (mm) (av.) | 5.07 | 5.10 | 5.07 | 5.07 |
| Hardness^{b} (N) (av.) | 244.0 | 229.8 | 239.4 | 231.0 |
| Disintegration time^{b} | 7:53 | 6:20 | 7:23 | 6:47 |
| (minutes:seconds) (av.) | | | | |
| Friability (%) | N/A | N/A | N/A | 0 |

The coating parameters (provided as ranges) for the spraying phase of the Opadry II coating are summarized in Table 16 below.

TABLE 16
Spray coating parameters for Opadry II coating.

| | |
|---|---|
| Air flow (m³/h) | 810-813 |
| Inlet air temperature (° C.) | 58-65 |
| Exhaust air temperature (° C.) | 43-48 |
| Spray rate (g/min) | 69-101 |
| Atomizing air (bar) | 2.5 |
| Pan speed (rpm) | 11.5-12.5 |
| Weight gain w/w (%) | 3.1 |
| Appearance (successful coating) | OK |

The experimental trials in small and scale up (30 Kg batch/45,000 tablets) batch sizes also confirm, robustness of the process. Generally, the physical attributes of the compressed tablets were acceptable, with no filming on the punches and acceptable final blend flowability, no rat-holding at larger scale, minimal batch to batch variability during dissolution.

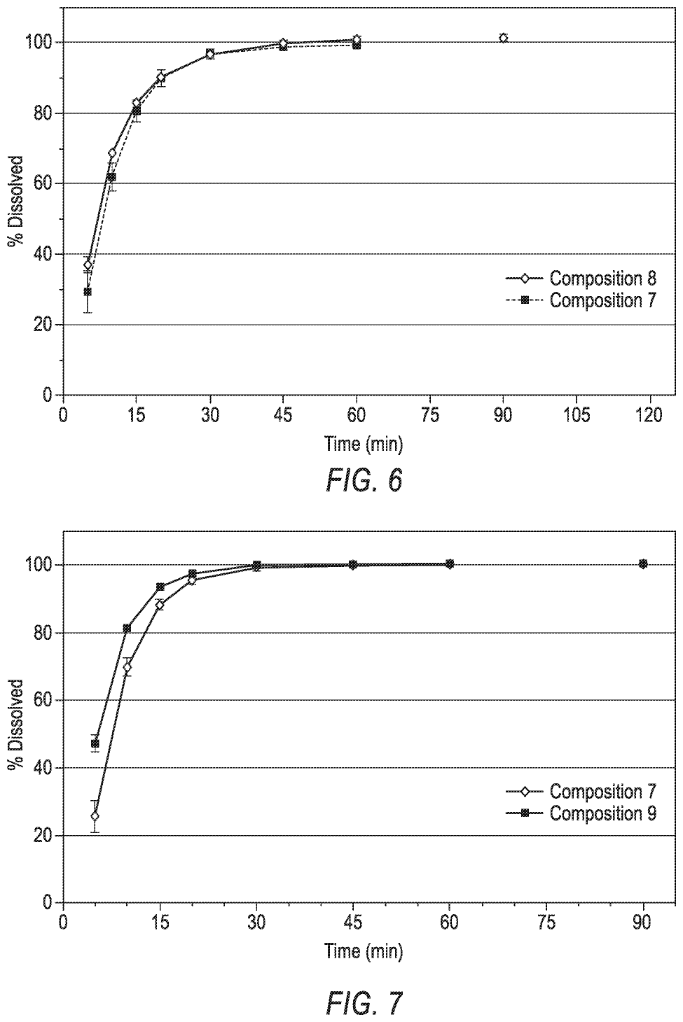

Dissolution experiments were also performed to compare the dissolution profiles of Tablet Compositions 7 and 8 using the QC methods described above. The dissolution profiles are presented in FIG. 6, which confirms that Tablet Composition 8 and Tablet Composition 7 possess similar dissolution profiles under the QC-4 dissolution method, which links to the CTAB—current QC lead method. The current QC lead method was developed due to the fact that at a certain time in development (between the development of the phase 2 formulation and phase 3 formulation) the QC-2 dissolution method was not well-suited for testing dissolution profiles of newly developed formulations that deviated from the Phase 2b formulation. The QC-2 dissolution method was developed for testing the dissolution properties of the Phase 2b formulation, which differs from other later developed formulations described herein, in terms of formulation and/or processing methods. Accordingly, it was desirable to further adapt the dissolution method to be useful for release and stability testing of later developed formulations which gave rise to QC-3 and QC-4 dissolution methods.

Improved Properties of Compositions 1-8

Compositions 1-8 constitute a vast improvement over the Phase 2b formulations described herein. The improved properties of Tablet Compositions 1-8 include 1) excellent flowability, 2) no detectable sticking or bridging during granulation process, and 3) no detectable filming during compression.

### Example 6: Process for Producing Tablet Composition 9 Having 300 mg Compound (1) (Molar Equivalent of the HCl Salt)

It was demonstrated that a tablet formulation having added external lubrication as well as less lubricant compound in the formulation itself would not hinder tablet manufacturing and production. Sodium Stearyl Fumarate (SSF) was used both in the external phase (see Table 17A below) and as a process aid. As a process aid SSF is sprayed externally to the punches and dies via an External Lubrication System that is connected to the tablet press for lubrication during compression and ejection. The overage of SSF applied with the External Lubrication System is removed by vacuum and thus does not affect formula composition.

TABLE 17A
Tablet Composition 9.

| | | |
|---|---|---|
| | | Wt % of total |
| Component (Function) | Mg/unit | core tablet |

Intragranular Phase

| | | |
|---|---|---|
| Compound (1) HCl hemihydrate salt | 334.20 | 51.42 |
| Form A (API) | | |
| Crospovidone (disintegrant) | 9.75 | 1.50 |
| Purified water (solvent) | 410.0^{a} | |
| Hypromellose (HPMC) 2910 15 mPa · s | 12.35 | 1.90 |
| (binder) | | |
| Polysorbate 20 (Tween 20) (wetting | 3.25 | 0.50 |
| agent) | | |
| Total Intragranular phase | 359.55 | |

Extragranular phase

| | | |
|---|---|---|
| Silicified microcrystalline cellulose | 170.20 | 26.18 |
| (SMCC HD90) (diluent) | | |
| Colloidal anhydrous silica (glidant) | 6.50 | 1.00 |
| Microcrystalline cellulose (Ceolus) | 32.50 | 5.00 |
| (diluent) | | |
| Partially gelatinized maize starch | 32.50 | 5.00 |
| (Starch 1500) (diluent) | | |
| Crospovidone (disintegrant) | 35.75 | 5.50 |
| Sodium stearyl fumarate (lubricant) | 13.00 | 2.00 |
| Total EF | 290.45 | |
| Total Core Tablet^{b} | 650.00 | 100 |

Film Coating

| | | |
|---|---|---|
| Opadry II Yellow 85F92450 (Coating | 19.50 | +3.00 |
| powder) | | |
| Purified water (solvent) | 78.00^{a} | |

^{a}Removed during processing.
^{b}Tablets are coated to a target coating weight of 3%.

Compression parameters and physical attributes of the tablet cores of Tablet Composition 9 are provided in Table 17B below.

TABLE 17B
Compression parameters and physical attributes of Composition 9.

| Parameter or | Comp. 9 | Comp. 9 | Comp. 9 | Comp. 9 | Comp. 9 | Comp. 9 |
|---|---|---|---|---|---|---|
| Result | (1) | (2) | (3) | (4) | (5) | (6) |
| Compression | 1250 | 2167 | 2167 | 2167 | 2167 | 2167 |
| speed (tpm) | | | | | | |
| Ejection force | N/A | N/A | N/A | N/A | N/A | N/A |
| (daN) | | | | | | |
| Compression | 1250 | 1250 | 1250 | 1250 | 1250 | 1250 |
| force (daN) | | | | | | |
| Weight (mg) | 643.78 | 649.52 | 648.67 | 651.42 | 652.90 | 652.09 |
| (av.) | | | | | | |
| Thickness | 5.16 | 5.16 | 5.18 | 5.18 | 5.19 | 5.19 |
| (mm) (av.) | | | | | | |
| Hardness (N) | 216.5 | 231.2 | 226.7 | 233.3 | 234.6 | 232.1 |
| (av.) | | | | | | |
| Disintegration | 2 | 2 | 2 | 2 | 2 | 2 |
| time (min:sec) | | | | | | |
| (av.) | | | | | | |
| Friability (%) | 0.0 | 0.0 | 0.0 | 0.1 | 0.1 | 0.0 |

Improved Properties of the Compositions.

Dissolution experiments using the physiology based (PB) method of dissolution that compare the dissolution rates of Composition 7 to the Phase 2b composition, conclude that Composition 7 consistently has improved dissolution properties as compared to the Phase 2b composition (see FIG. 5).

Dissolution experiments were also performed to compare the dissolution profiles of Tablet Compositions 7 and 9 using the QC-4 dissolution method, as detailed above. These dissolution profiles are presented in FIG. 7, which confirms that Tablet Composition 9 disintegrated at a faster rate and presented a faster dissolution profile compared with the Tablet Composition 7 using the QC-4 dissolution method.

### Example 7: Process for Producing the Phase 2b Formulation

Compound (1) HCl hemihydrate salt Form A was employed for the Phase 2b formation. All excipients complied with the current monographs of the European Pharmacopoeia and the USP/NF and are purchased from approved suppliers.

Variation 1:

The formulation composition and batch size for the pre-granulation blend and the granulation binder solution are given in Tables 18A and 18B, respectively. The batch size of the binder solution included a 100% overage for pump calibration and priming of solution lines. The theoretical compression blend composition is also given in Table 18C. The actual quantities for the batch were calculated based on the yield of the dried granules. The composition and approximate batch size of the film coating suspension is given in Table 18D and included 100% overage for pump calibration and priming of suspension lines. The target amount of the film coating was 3.0% w/w of the tablet weight.

TABLE 18A
Compositions of Tablets of Compound (1).

| | | Quantity |
|---|---|---|
| | | per batch |
| Component | % w/w | (g) |
| Form A of the HCl salt of Compound (1) | 76.14 | 4874.76 |
| Avicel PH-101 (microcrystalline cellulose), | 10.03 | 642.01 |
| NF, PhEur, JP | | |
| Lactose Monohydrate, #316, NF, PhEur, JP | 10.03 | 642.01 |
| Ac-Di-Sol (croscarmellose sodium), NF, | 3.81 | 243.74 |
| PhEur, JP | | |
| Total | 100.00 | 6402.50 |

TABLE 18B
Binder solution composition.

| Component | % W/W |
|---|---|
| Povidone K30, USP | 3.6 |
| Water | 96.4 |
| Total | 100.00 |

TABLE 18C
Compression blend composition.

| Component | % W/W | Batch size (g)* |
|---|---|---|
| Compound (1) TSWG granulation | 66.67 | 6000.3000 |
| Avicel PH-102, NF, PhEur, JP | 26.83 | 2414.6708 |
| Ac-Di-Sol, NF, PhEur, JP | 2.50 | 225.0113 |
| Sodium Stearyl Fumarate, NF, PhEur, | 4.00 | 360.0180 |
| JP | | |
| Total | 100.00 | 9000.00 |

*Total batch size will depend on granulation yield and % of water in dried granules.

TABLE 18D
Film coat suspension composition and approximate batch size.

| Component | % W/W | Batch size (g) |
|---|---|---|
| Opadry II White, 33G | 15.00 | 210.00 |
| Water, USP | 85.00 | 1190.00 |
| Total | 100.00 | 1400.00 |

Variation 2:

Variation 2 of the Phase 2b formulation is presented in Table 19 below.

TABLE 19
Variation 2 of the Phase 2b formulation.

| | | Wt % in | |
|---|---|---|---|
| | | tablet | Mg in |
| Phase | Component | core | tablet |
| Intragranular | Compound (1) HCl hemihydrate | 50.00 | 333.00 |
| | salt Form A | | |
| | Avicel PH-101, NF, Ph. Eur. | 6.59 | 43.89 |
| | Lactose monohydrate, #316, | 6.59 | 43.89 |
| | NF, Ph. Eur. | | |
| | Ac-Di-Sol, NF, Ph. Eur., JP | 2.5 | 16.65 |
| | Total pre-granulation blend | 65.68 | 437.43 |
| Binder solution | Povidone K30, USP (in water) | 1.0 | 6.66 |
| | Total granules | 66.68 | 444.09 |
| Extragranular | Prosolv 50, NF | 28.82 | 191.94 |
| | Ac-Di-Sol, NF, Ph. Eur., JP | 2.50 | 16.65 |
| | SSF, NF | 2.00 | 13.32 |
| | Total core tablet | 100 | 666.00 |
| Film coating | Opadry II, 85F18422 | +3.2 | 21.31 |
| susp. | (in water) | | |
| | Total final coated tablet | | 687.31 |

Optimization of the Phase 2b Formulation.

The Phase 2b formulation was further optimized, varying several different variables according to Table 20 below. Tablet weights of the optimized Phase 2b formulations ranged from 670 mg to 1000 mg.

TABLE 20
Optimizations of the Phase 2b formulation.

| Phase | Component | Function | % |
|---|---|---|---|
| Intragranular | Compound (1) HCl hemi- | API | ±50 |
| | hydrate salt Form A | | |
| | SMCC (50-HD-90), MCC | Binder/filler | 8 |
| | (101-105) | | |
| | Lactose MH, Mannitol | Filler | 13.5-30 |
| | (160C, 100SD, 25) | | |
| | CPV, CCS | Disintegrant | 0-2 |
| | HPC/HPMC/PVP K30 | Binder (wet) | 0.6-1.2 |
| | Total intragranular | | 50-75 |
| Extragranular | SMCC (50-HD90), Lactose | Binder/filler | 20-22 |
| | MH, Microcelac, Pearlitol | | |
| | Flash | | |
| | CPV/CCS | Disintegrant | 3-5 |
| | Na Stearyl Fumarate - | Lubricant | 1.5-3.0 |
| | Mg Stearate | | |
| | Colloidal silicon dioxide | Glidant | 0-2 |
| | Talc | Other | 0-5 |

Table 21 below provides the complete composition of two optimized tablet core formulations in comparison with the original Phase 2b tablet composition.

TABLE 21
Comparison of optimized tablet core formulations to the Phase 2b formulation.

| | | Mg in | Mg in | Mg in |
|---|---|---|---|---|
| | | Phase | Opt. | Opt. |
| Phase | Component | 2b | Form. 1 | Form. 2 |
| Intragranular | Compound (1) HCl hemihydrate | 333.00 | 333.00 | 333.00 |
| | salt Form A | | | |
| | Avicel PH-101, NF, Ph. Eur. | 43.89 | 43.86 | 43.86 |
| | Lactose monohydrate, #316, | 43.89 | 43.86 | 43.86 |
| | NF, Ph. Eur. | | | |
| | Ac-Di-Sol, NF, Ph. Eur., JP | 16.65 | 16.65 | 16.65 |
| | Total pre-granulation blend | 437.43 | 437.37 | 437.37 |
| Binder solution | Povidone K30, USP (in water) | 6.66 | 6.66 | 6.66 |
| | Total granules | 444.09 | 444.03 | 444.03 |
| Extragranular | Prosolv 50, NF | 191.94 | 275.97 | 192.00 |
| | Ac-Di-Sol, NF, Ph. Eur., JP | 16.65 | — | 16.65 |
| | SSF, NF | 13.32 | — | 13.32 |
| | Aerosil | — | 7.50 | — |
| | Crospovidone | — | 15.00 | — |
| | Mg Stearate | — | 7.5 | — |
| | Total core tablet | 666.00 | 666.00 | 666.00 |

Procedure for Producing the Phase 2b Formulations.

Binder Solution Preparation.

The binder solution consisted of Povidone and water. The solution was prepared based on 40% water content in the final granulation. Thus, the total amount of solids in solution (Povidone) was 3.6% (w/w). An excess amount of 10000 was prepared for priming lines, etc. Based on visual inspection of startup of the granulation run, additional stock solutions of +/−2% (38-42%) water in the final granulation was prepared. Typically, 87.00 g Povidone K30, and 2320.00 g purified (DI) water were weighed, and under constant stirring was added the Povidone K30 into the container containing the DI water. After the addition, the container was sealed to minimize evaporation, and the solution was stirred until all the solids present were fully dissolved.

Wet Granulation Process Flow.

Wet granulation was performed by the procedures described below: Excess (10%) amount of Compound (1), Avicel PH-101, Fastflo lactose and croscarmellose sodium were weighed (see Table 14A). They were screened using a 20 mesh hand screen or a cone mill equipped with an 813 m grated mesh screen at 1000 rpm (for a U5 Quadro Co-mill). The screened materials were placed in individual bags or containers. The materials were then transferred into a blender, and were blended for 15 minutes at typically 15 rpm. The blended materials were milled using U5 Quadro cone mill equipped with 4 mm square hole screen at 1000 rpm. The milled materials were blended again, repeating the blend step. The re-blended materials were then fed into a twin screw granulator. The bulk wet granulation was fed into the granulator using a Loss in Weight feeder (K-tron or similar). The resulting materials were then granulated. The binder fluid (see Table 14A) was injected into the twin screw granulator using a peristaltic pump. The ratio of solution feed rate over powder feed rate was 0.4095. For example, if the powder feed rate was 15.00 g/min, the solution feed rate was 0.4095*15.00=6.14 g/min, with a water content of 40% (based on the dry mass). The granule sub batches were collected into pre-tared drying trays. The collected materials were evenly sprayed on a tray and dry the material in an oven to form dried granules. The dried granules were placed into K-tron to starve feed continuously into cone mill and subsequently milled.

Extra-Granular Blending and Compression Process.

Extra-granular blending and compression process were performed by the procedures described below: The quantity of the extra-granular excipients based on the compression blend composition was weighed. The weighed excipients were screened using a U5 Comil with a 32 C screen and round bar impeller at 1000 rpm. The milled granules of Compound (1) was first added to the blender containing the screened Avicel PH-102 and Ac-Di-Sol. They were blended for 8 minutes at 16 RPM. Sodium stearyl (SSF) was screened through a mesh 50 hand screen into an appropriate container. A portion of the extra granular blend equal to roughly 10 times by mass the amount of SSF was placed in the container with the SSF and bag blend for 30 seconds before adding the mixture to the bin blender. All of the materials were then blended for 2 minutes at 16 rpm. The final blend was then compressed according to the prescribed tablet compression process parameters.

Film Coating Process.

A film coating was applied to the core tablets in a Vector VPC 1355 pan coater as a 15% w/w Opadry II white #33G aqueous suspension. The target coating was 3.0% w/w of the core tablet weight, with an acceptable range of 2.5% to 3.5%. To accomplish this, an amount of coating suspension equivalent to a 3.2% weight gain was sprayed, which gave a 3.0% coating assuming a coating efficiency of 95%.

Performance of the Phase 2b Tablet Formulations.

The Phase 2b formula was originally processed using a modified 18 mm Leistritz hot melt twin screw extruder operating at room temperature. The inherent negative properties of the Phase 2b tablets include 1) poor flowability and high tendency of sticking and bridging during granulation process, and 3) filming during compression.

After multiple optimization attempts to improve the formulation and process of the Phase 2b tablet formulations (details above), the formulations still possessed negative properties such as 1) batch to batch variability in the stability behavior for dissolution, 2) poor final blend flowability and other properties, and 3) severe filming of punches during compression.

Due to the technical issues with the formulation/process, fluid bed granulation was selected as a platform for development to ensure the probability of success of producing the necessary clinical material and providing a robust and viable launch platform.

### Example 8: In Vivo Assay for Combination of Compound (1) with or without Oseltamivir

Infected mice were treated with vehicle or escalating dose levels of Compound (1) HCl hemihydrate salt Form A (hereinafter in this example simply Compound (1)) in combination with the clinically relevant dose of Oseltamivir starting 48 hours post influenza A challenge or 2 hours prior to Influenza B challenge.

Methods:

In these studies, Compound (1) was formulated in a vehicle containing 0.5% (w/v) MC (Sigma-Aldrich, St Louis, Mo.), yielding a homogeneous suspension, and the dose of the compound was based upon Compound (1). Oseltamivir was formulated in distilled deionized water yielding a homogeneous suspension. The combination of Compound (1) with oseltamivir was formulated in a vehicle containing 0.5% (w/v) MC. The combination formulations were prepared at the beginning of each study and stored at 4° C. for up to 10 days with stirring in the dark. All formulations and vehicles were administered to mice via oral gavage at a dosing volume of 10 mL/kg.

Male Balb/c mice (5-7 weeks, 17-19 grams) were anesthetized and inoculated with a lethal dose of mouse-adapted influenza virus A/PR/8/34 or B/Mass/3/66 by intranasal instillation. Eight mice were enrolled per study group. Treatments were initiated +48 hours post inoculation for influenza A or 2 hours prior to inoculation for influenza B. Vehicle (10 mL/kg) and Compound (1) at doses of 0.1-10 mg/kg was administered alone or in combination with 10 mg/kg Oseltamivir orally (PO) twice daily (BID) for 10 days in the influenza A study. Vehicle (10 mL/kg) and Compound (1) at doses of 1-10 mg/kg was administered alone or in combination with 10 mg/kg Oseltamivir orally (PO) twice daily (BID) for 10 days in the influenza B study. Mice were weighed and observed daily for signs of morbidity for 21 days after infection. In addition, lung function was monitored by unrestrained WBP (Buxco, Troy, N.Y.).

Influenza A/PR/8/34 (VR-1469) and Influenza B/Mass/3/66 (VR-523) were obtained from ATCC (Manassas, Va.). Stocks were prepared by standard methods known in the art. Briefly, virus was passaged at low multiplicity of infection in Madin-Darby canine kidney cells (MDCK cells, CCL-34, ATCC), the supernatant harvested after approximately 48 hours and centrifuged at 650×g for 10 minutes. Virus stocks were frozen at −80° C. until used. Virus titers (TCID₅₀/ml) were calculated by the Spearman-Karger method after serially diluting the virus sample, infecting replicate MDCK cultures, and measuring the cytopathic effect (CPE) based on ATP content at 96 hours (CellTiter-Glo, Promega, Madison Wis.).

Mice were weighed daily for 21 days after infection. Body weight data were analyzed using Two Way ANOVA and a Bonferroni post-test to compare groups. P-values less than 0.05 were considered significant.

Mice were observed daily for 21 days post influenza infection. Any mouse that scored positive for four of the following six observations (>35% body weight (BW) loss, ruffled fur, hunched posture, respiratory distress, reduced mobility, or hypothermia) was deemed moribund, then euthanized and scored as a death in accordance with guidelines established with the Vertex Institutional Animal Care and Use Committee. Survival data were analyzed using the Kaplan Meier method.

Mice were subjected to unrestrained WBP (Buxco, Troy, N.Y.). Lung function is expressed as enhanced pause (Penh), a unit-less calculated value that reflects pulmonary resistance. This value is derived from changes in the holding container pressure that fluctuates as a consequence of changes in the animal's breathing pattern. Bronchoconstriction of the animal's airways will affect the flow of air and, hence, pressure in the holding container. The changes in pressure are tracked during expiration (PEP) and inspiration (PIP). Penh values were calculated according to the formula Penh=pause×PEP/PIP, where “pause” reflects the timing of expiration. Mice were acclimated in the Plethysmography chamber for 15 minutes, then data were collected in one minute intervals, averaged over 10 minutes, and expressed as absolute Penh values. Data were analyzed using Two Way ANOVA and a Bonferroni post-test to compare groups. P-values less than 0.05 were considered significant.

Results:

Compound (1) was evaluated in combination with Oseltamivir for its ability to prevent mortality and morbidity, reduce BW loss, and prevent and/or restore lung function in a murine model of influenza pulmonary infection versus Compound (1) or Oseltamivir treatment alone. The combination showed no deleterious effect on the efficacy of each of the drugs as compared to each drug administered alone. In addition, the combination treatment showed synergy in influenza A treatment as the failure dose for each compound alone (0.3 and 10 mg/kg of Compound (1) and Oseltamivir, respectively) when combined increased survival from 0 to 100 percent. Compound (1) has little activity against influenza B in vivo (as expected from available in vitro data) and does not interfere with the effectiveness of Oseltamivir.

Influenza A Mouse Model:

All of the vehicle-treated controls succumbed to disease by days 9 or 10. Treatment at 1, 3 and 10 mg/kg Compound (1) BID alone provided complete protection from death, reduced BW loss and restored lung function when dosing was initiated +48 hours post infection as compared to vehicle controls (Table 22). Treatment at 0.1 and 0.3 mg/kg Compound (1) and 10 mg/kg Oseltamivir administered alone did not protect from death reduce BW loss or restore lung function when treatment initiated +48 hours post influenza A infection. 0.3 mg/kg Compound (1) and Oseltamivir administered together +48 hours post influenza A infection provided complete protection from death, reduced BW loss and restored lung function.

TABLE 22
In Vivo Efficacy Data of Compound (1) with or without
Oseltamivir Administered +48 Hours After Influenza A Infection.
Compound (1)/Oseltamivir Combination in FluA

| | Oseltamivir mg/kg | | | | | |
|---|---|---|---|---|---|---|
| | 0 | | | 10 | | |
| Compound | Survival | Weight Loss | Penh | Survival | Weight Loss | Penh |
| (1) mg/kg | (21 days) (%) | (Day 8) (%) | (Day 3) | (21 days) (%) | (Day 8) (%) | (Day 3) |
| 0 | 0 | 33.9 | 2.28 | 0 | 32.0 | 2.36 |
| 0.1 | 0 | 34.2 | 2.15 | 0 | 31.6 | 2.09 |
| 0.3 | 0 | 32.4 | 1.90 | 100 | 29.3 | 1.80 |
| 1 | 100 | 28.2 | 2.11 | 100 | 23.4 | 1.23 |
| 3 | 100 | 22.2 | 1.68 | 100 | 17.6 | 1.11 |
| 10 | 100 | 14.6 | 0.95 | 100 | 8.4 | 0.79 |

Influenza B Mouse Model:

All of the vehicle-treated controls succumbed to disease by days 7 or 8. Administration of 1, 3, or 10 mg/kg Compound (1) alone −2 h prior to influenza B infection and continued BID for 10 days provided no significant protection against morbidity, BW loss or loss of lung function as compared to controls. Oseltamivir administered at 10 mg/kg alone or in conjunction with 1, 3 or 10 mg/kg Compound (1) 2 hours prior to influenza B infection provided complete protection from death, reduced BW loss and restored lung function (Table 23).

TABLE 23
In Vivo Efficacy Data of Compound (1) with or without
Oseltamivir Administered +48 Hours After Influenza B Infection.
Compound (1)/Oseltamivir Combination in FluB

| | Oseltamivir mg/kg | | | | | |
|---|---|---|---|---|---|---|
| | 0 | | | 10 | | |
| Compound | Survival | Weight Loss | Penh | Survival | Weight Loss | Penh |
| (1) mg/kg | (21 days) (%) | (Day 8) (%) | (Day 6/7) | (21 days) (%) | (Day 8) (%) | (Day 6/7) |
| 0 | 0 | ND | 2.20 | 100 | 12.8 | 1.08 |
| 1 | 0 | 33.6 | 1.90 | 100 | 7.7 | 1.26 |
| 3 | 0 | 33.9 | 2.06 | 100 | 11.5 | 1.41 |
| 10 | 0 | 33 | 2.04 | 100 | 9.7 | 1.17 |

### Example 9: In Vivo Assay for Combination of Compound (1) with Zanamivir

Infected mice were treated with vehicle or escalating dose levels of Form A of the Compound (1) HCl hemihydrate salt Form A (hereinafter in this example simply Compound (1)) in combination with zanamivir starting 24 hours prior to influenza A challenge with 5×10³ TCID₅₀ A/PR/8/34. The influenza A challenge and Compound (1) suspensions were prepared in a similar manner as described above in Example 8. The challenged mice were treated once IN (intranasal) with zanamivir at 0.3 mg/kg, 1 mg/kg or 3 mg/kg 24 hours prior to IN challenge with 5×10³ TCID₅₀ A/PR/8/34, and with Compound (1) at 0.1 mg/kg, 0.3 mg/kg, or 1 mg/kg BID for 10 days starting −2 hours prior to the challenge with 5×10³ TCID₅₀ A/PR/8/34.

The results are summarized in Tables 24A and 24B below. As shown in Tables 24A below, the combination therapy with Compound (1) and zanamivir provided extra survival benefit. Efficiency quotient, a composite measure of survival, bodyweight loss and lung function (% survival/(% body weight loss at Day 8)*(Penh at Day 6)) is summarized in Table 24B.

TABLE 24A
Survival Rate: Combination Therapy of
Compound (1) with Zanamivir.

Compound (1) (mg/kg, BID)
1^{st} dose 2 h prior to infection

| | | | 0.1 | 0.3 | 1 |
|---|---|---|---|---|---|
| Zanamivir (mg/kg, | 0 | 0 | 12.5 | 44.4 | 100 |
| IN × 1), 1^{st} | 0.3 | 37.5 | 0 | 100 | 100 |
| dose 24 h prior | 1 | 50 | 75 | 100 | 100 |
| to infection | 3 | 62.5 | 100 | 100 | 100 |

TABLE 24B
Efficiency Quotient: Combination Therapy of
Compound (1) with Zanamivir.

Compound (1) (mg/kg, BID)
1^{st} dose 2 h prior to infection

| | | | 0.1 | 0.3 | 1 |
|---|---|---|---|---|---|
| Zanamivir (mg/kg, | 0 | — | — | 0.59 | 2.32 |
| IN × 1), 1^{st} | 0.3 | 0.44 | — | 1.35 | 2.97 |
| dose 24 h prior | 1 | 0.73 | 1.00 | 1.61 | 2.31 |
| to infection | 3 | 0.73 | 1.30 | 1.48 | 4.28 |

### Example 10: Prophylactic and Post-Infection Efficacy of Compound (1) in the Mouse Influenza A Infection Model

Materials and Methods

Animals:

Female 18-20 g BALB/c mice were obtained from Jackson Laboratories (Bar Harbor, Me.) for the antiviral experiment. The animals were maintained on standard rodent chow and tap water ad libitum. They were quarantined for 48 hours prior to use.

Virus:

Mouse-adapted Influenza A/California/04/2009 (pndH1N1) virus was obtained from Dr. Elena Govorkova (St. Jude Children's Research Hospital, Memphis, Tenn.). The virus stock was amplified in MDCK cells, followed by titration for lethality in BALB/c mice. Influenza A/Victoria/3/75 (H3N2) virus was obtained from the American Type Culture Collection (Manassas, Va.). The virus was passaged seven times in mice to mouse-adapt it, followed one passage in MDCK cells. The virus was further titrated for lethality in BALB/c mice to obtain the proper lethal challenge dose. Influenza A/Vietnam/1203/2004 (H5N1) virus was obtained from Dr. Jackie Katz of Centers for Disease Control (Atlanta, Ga.). Mice were exposed to a lethal dose of the virus (5 MLD50, 5 PFU/mouse), which has previously resulted in death between days 6-13, with 90-100% mortality by day 10 at this dose.

Compounds:

Oseltamivir (as Tamiflu®) was obtained from a local pharmacy. Each capsule of Tamiflu contains 75 mg of the active component, oseltamivir carboxylate, upon metabolism in the body. The dose of oseltamivir was based upon this measurement. Compound (1) HCl hemihydrate salt Form A (hereinafter in this example simply Compound (1)) was for the study and the dose of the compound was based upon the HCl salt of Compound (1) hemihydrate. Both Compound (1) and oseltamivir were prepared in 0.5% methylcellulose (Sigma, St. Louis, Mo.) for oral gavage (p.o.) administration to mice.

Experiment Design:

The mice were anesthetized by intraperitoneal injection of ketamine/xylazine (50/5 mg/kg), and the animals were infected intranasally with a 90-μl suspension of influenza virus. The virus challenge was approximately four 50% mouse lethal infectious doses. Treatments were given twice a day (at 12 hours intervals) for 10 days starting 2 hours before virus challenge or up to 48 hours post challenge as indicated. Parameters for assessing the infection were survival, mean day of death, body weight changes, and lung infection parameters (hemorrhage score, weight, and virus titer). Animals were weighed individually every other day through day 21 of the infection. Mice that died during the first six days of treatment period were deemed to have died from causes other than influenza virus infection, and were excluded from the total counts.

To assess lung infection parameters, lungs from sacrificed animals (initially 5 animals per group set apart for this purpose) were harvested. Lung hemorrhage score was assessed by visual inspection for color changes from pink to plum. This occurs regionally in the lungs, rather than by a gradual change of the whole lung to the darker color. Hemorrhage scores ranged from 0 (normal) to 4 (total lung showing plum color), and thus is a non-parametric measurement. The lungs were weighed and then frozen at −80° C. Later, thawed lungs were homogenized in 1 ml of cell culture medium, the supernatant fluids were centrifuged to remove particulate matter, and the liquid samples were re-frozen at −80° C. After preparing 96-well plates of MDCK cells, the samples were thawed, serially diluted in 10-fold dilution increments and titrated by endpoint dilution method in the plates (1), using 4 microwells per dilution. Virus titers were calculated as log 10 50% cell culture infectious doses per gram of lung tissue (log 10 CCID50/g).

Statistical Analysis:

Kaplan-Meir plots for multiple group comparisons were analyzed by the Mantel-Cox log-rank test to determine statistical significance. Subsequently, pairwise comparisons were made by the Gehan-Breslow-Wilcoxon test. The relative experimental significance was adjusted to a Bonferroni corrected significance threshold based on the number of treatment comparisons made. Mean day of death and mean lung hemorrhage score comparisons were analyzed by the Kruskal-Wallis test followed by Dunn's multiple comparisons test. Mean body weights, lung weights, and log 10 lung virus titers were evaluated by ANOVA assuming equal variance and normal distribution. Following ANOVA, individual treatment values were compared by the Tukey-Kramer multiple comparisons test. Analyses were made using Prism® software (GraphPad Software, San Diego, Calif.).

Results and Discussions

The prophylactic dose response of Compound (1) was investigated in the mouse influenza A model. Dosing with vehicle or Compound (1) was initiated 2 h prior to infection and continued twice daily for 10 days. The results are summarized in Tables 20 and 21. All of the mice that received vehicle alone succumbed to the infection by study day 9 and had lost, on average, ˜32% of their body weight (BW). Compound (1) administered at 1, 3 or 10 mg/kg BID provided complete survival and a dose-dependent reduction in BW loss. Compound (1) administered at 0.3 mg/kg BID provided some survival benefit (2/8 mice) although the mice had significant BW loss. In the same experiment, mice were dosed with oseltamivir at 10 mg/kg BID, a clinically-equivalent human dose (based on AUC). All of the oseltamivir-administered mice survived with a similar weight loss profile to mice administered 1 mg/kg BID Compound (1). Compound (1) still provided effectiveness in this model challenged with Influenza A/Vietnam/1203/2004 (H5N1) virus when it was administered at 48 hours post infection, with continued BID dosing for 10 days (Table 25). Dosing of Compound (1) at 10 mg/kg provided complete protection as shown in Table 26.

TABLE 25
Effects of Prophylaxis with Compound (1) and Oseltamivir
on an Influenza A/California/04/2009 (pndH1N1) Virus
Infection in BALB/c mice (prophylaxis).

| | | | Mean Lung Parameters (Day 6) | | |
|---|---|---|---|---|---|
| Compound | | | | Weight | Virus |
| (mg/kg)^{a} | Survivors/Total | MDD^{b} ± SD | Score | (mg) | Titer^{c} |
| Compound (1) | 10/10*** | — | 0.2 ± 0.4** | 132 ± 20*** | <2.6^{d}*** |
| (10 mg/kg) | | | | | |
| Compound (1) | 9/9*** | — | 0.0 ± 0.0*** | 123 ± 21*** | 3.1 ± 0.9*** |
| (3 mg/kg) | | | | | |
| Compound (1) | 10/10*** | — | 0.6 ± 0.9^{e} | 246 ± 21* | 5.5 ± 1.2*** |
| (1 mg/kg) | | | | | |
| Oseltamivir | 10/10*** | — | 1.0 ± 0.0^{e} | 178 ± 28*** | 7.9 ± 0.2 |
| (10 mg/kg) | | | | | |
| Placebo | 2/20 | 9.9 ± 1.3 | 3.4 ± 0.5 | 282 ± 26 | 7.9 ± 0.4 |

^{a}Dose per treatment, given twice a day for 10 days starting 2 hours prior to virus exposure.
^{b}Mean day of death of mice that died on or before day 21.
^{c}Log10 CCID50/g.
^{d}Below limit of detection (2.6 log10).
^{e}Not significant by the very stringent Dunn's multiple comparison test, but significant from placebo (P < 0.01) by the pairwise two-tailed Mann-Whitney U-test.
*P < 0.05,
**P < 0.01,
***P < 0.001, compared to placebo.

TABLE 26
Effects of Compound (1) and Oseltamivir on an Influenza A/Victoria/3/75
(H3N2) Virus Infection in BALB/c mice (prophylaxis).

| | | | Mean Lung Parameters (Day 6) | | |
|---|---|---|---|---|---|
| Compound (mg/kg)^{a} | Survivors/Total | MDD^{b} ± SD | Score | Weight (mg) | Virus Titer^{c} |
| Compound (1) | 10/10*** | — | 0.1 ± 0.2^{d} | 164 ± 11** | 6.1 ± 0.5*** |
| (10 mg/kg) | | | | | |
| Compound (1) | 10/10*** | — | 3.3 ± 0.6^{e} | 260 ± 25 | 7.2 ± 0.2 |
| (3 mg/kg) | | | | | |
| Compound (1) | 4/10 | 9.8 ± 1.9 | 3.2 ± 0.3^{e} | 274 ± 49 | 7.3 ± 0.3 |
| (1 mg/kg) | | | | | |
| Oseltamivir | 9/10*** | 7.0 | 1.7 ± 1.1 | 218 ± 24 | 7.0 ± 0.3** |
| (10 mg/kg) | | | | | |
| Placebo | 3/20 | 9.8 ± 2.1 | 2.2 ± 0.6 | 264 ± 54 | 7.8 ± 0.4 |

^{a}Dose per treatment, given twice a day for 10 days starting 2 hours prior to virus exposure.
^{b}Mean day of death of mice that died on or before day 21.
^{c}Log10 CCID50/g.
^{d}Not significant by the very stringent Dunn's multiple comparison test, but significant from placebo
(P < 0.01) by the pairwise two-tailed Mann-Whitney U-test.
^{e}Same as footnote “d”, but significant from placebo at P < 0.05 level.
**P < 0.01,
***P < 0.001, compared to placebo.

TABLE 27
Effects of Treatment (+48 h) with Compound (1) and Oseltamivir on
an Influenza A/Vietnam/1203/2004 (H5N1) Virus Infection in BALB/c mice.

| | | | Mean Lung Parameters (Day 6) | |
|---|---|---|---|---|
| Compound | | | Weight | Virus |
| (mg/kg)^{a} | Survivors/Total | MDD^{b} ± SD | (mg) | Titer^{c} |
| Compound (1) | 10/10 | >21 | 0.15 ± 0.02 | 3.75 ± 0.94 |
| (10 mg/kg) | | | | |
| Oseltamivir | 0/10 | 9.5 ± 1.2 | 0.17 ± 0.02 | 5.22 ± 0.38 |
| (10 mg/kg) | | | | |
| Placebo | 0/20 | 9.9 ± 0.8 | 0.16 ± 0.02 | 4.65 ± 1.23 |

^{a}Dose per treatment, given twice a day for 10 days starting 2 hours prior to virus exposure.
^{b}Mean day of death of mice that died on or before day 21.
^{c}Log10 CCID50/g.

### Example 11: In Vitro Efficacy of Compound (1) Against a Span of Influenza Strains

Cells and Viruses.

Madine Darby Canine Kidney (MDCK) cells were originally obtained from American Type Culture Collection (ATCC, Manassas, Va.) and passaged using standard laboratory techniques prior to use in infection assays. Cells were maintained at 37° C. in Dulbecco's modified Eagle's medium (DMEM; Invitrogen, Carlsbad, Calif.) supplemented with 10% fetal bovine serum (Sigma-Aldrich, St. Louis, Mo.), 2 mM L-glutamine, 10 mM HEPES, 100 U/mL penicillin and 100 ug/mL streptomycin (Invitrogen). Influenza virus was obtained from ATCC, the Virus Surveillance and Diagnosis Branch of the Influenza Division of the Centers for Disease Control and Prevention (CDC; Atlanta, Ga.) or the Influenza Reagent Resource, Influenza Division, WHO Collaborating Center for Surveillance, Epidemiology and Control of Influenza, CDC. To generate viral stocks, MDCK cells were infected with a low multiplicity of infection (MOI) in DMEM supplemented with 2 mM L-glutamine, 10 mM HEPES, 100 U/mL penicillin, 100 ug/mL streptomycin and 1 μg per mL tosylphenylalanyl chloromethyl ketone (TPCK)-treated trypsin (USB Corp.; Santa Clara, Calif.). Cells were incubated at 37° C. with 5% CO₂ for 48 h, after which time the supernatant was harvested by centrifugation at 900×g for 10 min with a Beckman GS-6R centrifuge. Virus stocks were aliquoted and frozen at −80° C.

Compounds.

Free base or Compound (1) HCl hemihydrate salt Form A (hereinafter in this example simply Compound (1)) was dissolved in 100% dimethyl sulfoxide (DMSO) to make a solution of a concentration of 10 mM.

Antiviral Activity.

The antiviral activity of Compound (1) and amantadine was evaluated in MDCK cells as measured by ATP levels using CellTiter-Glo (Promega; Madison, Wis.). MDCK cells were plated into black, clear bottom, 384-well plates to a density of 2×10⁴ cells per well in 50 μL VGM. Cells were incubated at 37° C., 5% CO₂, in saturated humidity to allow cells to adhere and form a monolayer. After 5 h, 40 μL of media was removed and 15 μL of virus was added at an MOI of 0.005. Compound was added as 25 μL of a ten point, three-fold dilution in DMEM with supplements (final DMSO concentration of 0.5%). Internal controls consisted of wells containing cells only and untreated cells infected with virus. After a 72 h incubation, 20 μL of CellTiter-Glo was added to each well and incubated at room temperature for 10 min. Luminescence was measured using an EnVision Multilabel reader (PerkinElmer; Waltham, Mass.). EC₅₀ values (concentration of compound that ensures 50% cell viability of uninfected control) were calculated by fitting the compound dose versus response data using a 4-parameter curve fitting method employing a Levenburg Marquardt algorithm (Condoseo software; Genedata, Basel, Switzerland). In vitro testing of hpaiH5N1 was performed at Southern Research Institute under BSL-3 containment.

As shown in Table 28 below, Compound (1) showed potent activity against all influenza A strains tested, including H1N1 and H3N2 reference strains from 1934 to 2009, as well as the pandemic 2009 H1N1 strains A/California/07/2009, A/Texas/48/2009, and the highly pathogenic avian H1N1 strain A/VN/1203/2004. Compound (1) was equally effective against all strains including those that were resistant to amantadine and neuraminidase inhibitors. It showed limited activity against influenza B virus.

TABLE 28
Efficacy of Compound (1) Against a Panel of Influenza Strains.

| | | | Cell Protection |
|---|---|---|---|
| | Inf. | | Assay^{e} |
| | Virus | | EC₅₀ ± SD |
| Influenza Strain | Strain | Subtype | Comp (1) (nM) |
| A/WS/33 ^{a} | A | H1N1 | 3.2 ± 4.3 |
| A/NWS/33 ^{a} | A | H1N1 | 0.73 ± 0.10 |
| A/Puerto Rico/8/34 ^{a} | A | H1N1 | 3.2 ± 1.8 |
| A/Weiss/43 ^{a} | A | H1N1 | 0.31 ± 0.23 |
| A/FM/1/47 | A | H1N1 | 0.57 ± 0.036 |
| A/Mal/302/54 | A | H1N1 | 0.57 ± 0.055 |
| A/Denver/1/57 | A | H1N1 | 0.42 ± 0.19 |
| A/Chelyabinsk/1/2006 | A | H1N1 | 0.70 ± 0.49 |
| A/Florida/3/2006 | A | H1N1 | 0.92 ± 1.5 |
| A/Fukushima/141/2006 | A | H1N1 | 0.18 ± 0.20 |
| A/Georgia/17/2006 | A | H1N1 | 0.13 ± 0.048 |
| A/Georgia/20/2006^{b} | A | H1N1 | 2.6 ± 3.8 |
| A/Missouri/3/2006 | A | H1N1 | 0.21 ± 0.060 |
| A/St. Petersburg/8/2006 ^{a} | A | H1N1 | 0.88 ± 0.69 |
| A/Virginia/01/2006 ^{a} | A | H1N1 | 0.42 ± 0.24 |
| A/Cambodia/0371/2007 ^{a}* | A | H1N1 | 0.61 ± 0.33 |
| A/South Dakota/6/2007 | A | H1N1 | 0.31 ± 0.25 |
| A/California/07/2009 NYMC | A | H1N1 | 2.7 ± 1.8 |
| X-179A ^{a} | | | |
| A/Aichi/2/68 | A | H3N2 | 1.4 ± 1.1 |
| A/Hong Kong/8/68 | A | H3N2 | 0.60 ± 0.11 |
| A/Port Chalmers/1/73 ^{a} | A | H3N2 | 0.54 ± 0.11 |
| A/Victoria/3/75 | A | H3N2 | 1.3 ± 0.63 |
| A/Wisconsin/67/2005 ^{a} | A | H3N2 | 1.8 ± 0.24 |
| A/Hawaii/2/2006 | A | H3N2 | 1.4 ± 0.91 |
| A/Nebraska/1/2006 ^{a}* | A | H3N2 | 2.1 ± 1.3 |
| A/Texas/12/2007 ^{a}*^{c} | A | H3N2 | 0.65 ± 0.22 |
| A/Uruguay/716/2007 ^{a} | A | H3N2 | 3.5 ± 5.1 |
| A/New Jersey/8/76 | B | H1N1 | 0.20 ± 0.096 |
| A/California/07/2009 ^{a} | C | H1N1 | 1.8 ± 1.6 |
| A/Mexico/4108/2009 ^{a} | C | H1N1 | 2.7 ± 1.8 |
| A/New York/18/2009 ^{a}* | C | H1N1 | 0.59 ± 0.40 |
| A/Texas/48/2009^{b} | C | H1N1 | 2.8 ± 3.2 |
| A/Virginia/ATCC2/2009 | C | H1N1 | 1.9 ± 3.0 |
| A/Virginia/ATCC3/2009 | C | H1N1 | 1.9 ± 3.2 |
| A/Swine/Iowa/15/30 | C | H1N1 | 0.65 ± 0.082 |
| A/Swine/1976/31 | C | H1N1 | 0.47 ± 0.11 |
| A/Equine/2/Miami/63 | C | H3N8 | 0.50 ± 0.065 |
| A/Viet Nam/1203/2004 ^{a} | K | H5N1 | <1.5 ± ND |
| B/Lee/40 | | | >10 ± ND |
| B/Russia/69 | | | >10 ± ND |

^{a} amantadine resistance: M2 31N mutation.
^{b}oseltamivir carboxylate resistance: NA 275Y mutation.
^{c} oseltamivir carboxylate resistance: NA 119V mutation.
*externally validated phenotypic resistance, sequence data unavailable.

### Example 12: In Vitro Combination Experiments with Compound (1) and Oseltamivir, Zanamivir, or Favipiravir

A solution of Compound (1) (free base or Compound (1) HCl hemihydrate salt Form A (hereinafter in this example simply Compound (1)) dissolved in 100% dimethyl sulfoxide (DMSO) was tested in a three day MDCK cell CPE-based assay, infected with A/Puerto Rico/8/34 at an MOI of 0.01, in combination experiments with either the neuraminidase inhibitors oseltamivir carboxylate and zanamivir, or the polymerase inhibitor T-705. Oseltamivir carboxylate and T-705 were dissolved in 100% dimethyl sulfoxide (DMS); zanamivir was dissolved in Dulbecco's modified eagle medium (DMEM) at a concentration of 10 mM and stored at −20° C. The study employed either the Bliss independence method (Macsynergy) (e.g., Prichard, M. N. and C. Shipman, Jr., Antiviral Res, 1990. 14 (4-5): p. 181-205) or the Loewe additivity/Median-effect method (e.g., Chou, T. C. and P. Talalay, Adv Enzyme Regul, 1984. 22: p. 27-55). The Bliss independence method involves testing different concentration combinations of inhibitors in a checkerboard fashion, while the Loewe independence method involves testing a fixed ratio combination of inhibitors, at different dilutions of the fixed ratio. Experiments were also performed using combinations of Compound (1) with itself as a control, confirming additivity. Cell viability was determined using CellTiter-Glo.

The Bliss independence method resulted in synergy volumes of 312 and 268 for oseltamivir carboxylate and zanamivir, respectively; and a synergy volume of 317 was obtained for favipiravir. Synergy volumes greater than 100 are generally considered strong synergy and volumes between 50 and 100 are considered moderate synergy. The Loewe additivity method produced C.I. (combination index) values of 0.58, 0.64, and 0.89 at the 50% effect level for oseltamivir, zanamivir, and T-705, respectively. C.I. values of less than 0.8 are considered strong synergy while values between 0.8 and 1.0 are considered additive to mildly synergistic. These data together, as shown in Table 29, suggest that Compound (1) is synergistic with the neuraminidase inhibitors and polymerase inhibitor tested.

TABLE 29
Summary of In Vitro Synergy and Antagonism Experiments.

| | Combination Index | | | |
|---|---|---|---|---|
| Loewe Additivity | ED₅₀ | ED₇₅ | ED₉₀ | Result |
| Compound (1) + oseltamivir | 0.60, 0.56 | 0.57, 0.56 | 0.59, 0.58 | Strong synergy |
| Compound (1) + zanamivir | 0.68, 0.61 | 0.67, 0.66 | 0.71, 0.77 | Strong synergy |
| Compound (1) + favipiravir | 0.83, 0.96 | 0.76, 1.0 | 0.71, 1.1 | Additivity to |
| | | | | weak synergy |

| | Synergy Volume, | |
|---|---|---|
| Bliss Independence | 95% Confidence | Result |
| Compound (1) + oseltamivir | 312 | Strong synergy |
| Compound (1) + zanamivir | 268 | Strong synergy |
| Compound (1) + favipiravir | 317 | Strong synergy |

ED₅₀, ED₇₅, ED₉₀: Compound concentration at which 50%, 75%, or 90%, respectively, of cells are Protected; Combination indexes were calculated at the effect levels of ED₅₀, ED₇₅ and ED₉₀.

### Example 13: Efficacy in the Mouse Influenza A Infection Model

The prophylactic dose response of Compound (1) (in amorphous form or Compound (1) HCl hemihydrate salt Form A (hereinafter in this example simply Compound (1)) was investigated in the mouse influenza A model. Dosing with vehicle or Compound (1) was initiated 2 h prior to infection and continued twice daily for 10 days. All of the mice that received vehicle alone succumbed to the infection by study day 9 and had lost, on average, ˜32% of their body weight (BW). Compound (1) administered at 1, 3 or 10 mg/kg BID provided complete survival and a dose-dependent reduction in BW loss. Compound (1) administered at 0.3 mg/kg BID provided some survival benefit (2/8 mice) although the mice had significant BW loss. In the same experiment, mice were dosed with oseltamivir at 10 mg/kg BID, a clinically-equivalent human dose (based on AUC). All of the oseltamivir-administered mice survived with a similar weight loss profile to mice administered 1 mg/kg BID Compound (1).

The extent to which Compound (1) administration could be delayed and still provide effectiveness in this model was investigated by challenging mice with influenza A virus and dosing with vehicle, oseltamivir, or Compound (1) starting at 24, 48, 72, 96 or 120 h post infection, with continued BID dosing for 10 days (Table 27). All vehicle controls succumbed to disease by study days 8 or 9. Compound (1) administered at 1, 3 or 10 mg/kg BID provided complete protection from death and reduced BW loss when dosing was initiated up to 72 h post infection compared with vehicle controls. Dosing of oseltamivir at 10 mg/kg BID only provided complete protection when dosing was initiated 24 h or less, post infection. When initiation of compound administration was delayed further, Compound (1) at 3 or 10 mg/kg BID provided complete survival at 96 h post infection and partial protection when initiation of dosing was delayed 120 h post infection.

The effectiveness of Compound (1) to reduce lung viral titers was investigated. Mice were infected with influenza A and 24 h later vehicle, oseltamivir (10 mg/kg BID) or Compound (1) (3, 10, 30 mg/kg BID) was administered until lung harvest and viral burden determination on day 6 (Table 27). All Compound (1)-administered groups showed robust, statistically significant reductions in lung viral titers compared with oseltamivir- and vehicle-administered animals.

In order to establish a PK/PD model, mice were infected with influenza virus for 24 h and then administered Compound (1) for an additional 24 h. Doses were fractionated as a single dose, two or four doses administered every 12 h or 6 h, respectively. Lungs and plasma were collected to determine lung viral loads and Compound (1) concentrations. The individual lung titer data from these dosing regimens (q6h, g12h and q24h) was plotted against individual Cₘₐₓ, Cₘᵢₙ, or AUC values (data not shown). While there was a clear correlation between lung titer reduction and Cₘᵢₙ, there was little correlation with Cₘₐₓ and only a weak correlation with AUC. There was a strong correlation with Cₘᵢₙ when the measured Compound (1) concentrations in plasma was plotted versus the measured lung titers. The half maximal reduction in lung titers (2-3 log) occurs near the serum-shifted EC₉₉ (100 ng/mL). A similar correlation was found between lung titer and measured Compound (1) concentrations in the lungs (data not shown).

TABLE 30
Summary of Percent Survival and Percent Body
Weight Loss in Mouse Model of Influenza A.

| Treatment Start Time | Compound (1) Dose | Oseltamivir Dose | Percent | Percent Body Weight |
|---|---|---|---|---|
| Relative Infection (h) | (mg/kg; BID) | (mg/kg; BID) | Survival | Loss on Study Day 8 |
| −2 | 10 | | 100 | −2.8 |
| | 3 | | 100 | −8.7 |
| | 1 | | 100 | −16.8 |
| | 0.3 | | 25 | −30.4 |
| | 0.1 | | 0 | −31.9 |
| | | 10 | 100 | −19.1 |
| | 0 | | 0 | −32.2 |
| +24^{a} | 10 | | 100 | −6.2 |
| | 3 | | 100 | −14.2 |
| | 1 | | 100 | −23.4 |
| | | 10 | 100 | −28.9 |
| | 0 | | 0 | −33.8 |
| +48^{a} | 10 | | 100 | −7.1 |
| | 3 | | 100 | −10.9 |
| | 1 | | 100 | −22.5 |
| | | 10 | 80 | −31.1 |
| | 0 | | 0 | −34.4 |
| +72^{a} | 10 | | 100 | −17.4 |
| | 3 | | 100 | −23.2 |
| | 1 | | 100 | −29.4 |
| | | 10 | 0 | −31.3 |
| | 0 | | 0 | −36.1 |
| +96^{b} | 10 | | 100 | −25.5 |
| | 3 | | 100 | −27.3 |
| | | 10 | ND^{c} | ND^{c} |
| | 0 | | 0 | −34.6 |
| +120^{b} | 10 | | 37.5 | −34.4 |
| | 3 | | 12.5 | −32.6 |
| | | 10 | ND^{c} | ND^{c} |
| | 0 | | 0 | −34.6 |

^{a}Data are from independent experiments.
^{b}Data are from the same experiment.
^{c}ND, not determined.

TABLE 31
Summary of Lung Viral Titer and Log₁₀ Reduction
in Mouse Model of Influenza A.

| | Study 1 | | Study 2 | |
|---|---|---|---|---|
| | Lung Viral | Log₁₀ | Lung Viral | Log₁₀ |
| | Titer | Reduction | Titer | Reduction |
| Treatment^{a} | (Log₁₀ TCID₅₀)^{b} | vs. Vehicle | (Log₁₀ TCID₅₀)^{b} | vs. Vehicle |
| 10 mg/kg BID | 6.20 | | 6.28 | |
| Vehicle | | | | |
| 10 mg/kg BID | 6.05 | −0.15 | | |
| Oseltamivir | | | | |
| 30 mg/kg BID | 3.95 | −2.25*** | 4.53*** | −1.75 |
| Compound (1) | | | | |
| 10 mg/kg BID | | | 5.20*** | −1.08 |
| Compound (1) | | | | |
| 3 mg/kg BID | | | 5.24*** | −1.04 |
| Compound (1) | | | | |

^{a}Animal Treatment was initiated 24 houses post infection and continued for 5 days.
^{b}Lung viral titers were determined on study day 6.
^{c}ND, not determined.
2 way ANOVA with Bonferroni Post Test,
***P < 0.001.

### Example 14: Proof-of-Concept Influenza Challenge

A live, attenuated influenza challenge model was used previously to predict the effectiveness of influenza antivirals in natural infection in humans (Calfee, D. P., Peng, A. W., Hussey, E. K., Lobo, M. & Hayden F. G. Safety and efficacy of once daily intranasal zanamivir in preventing experimental human influenza A infection. Antivir Ther. 4, 143-149 (1999); Hayden, F. G. et al. Use of the oral neuraminidase inhibitor oseltamivir in experimental human influenza. JAMA 282, 1240-1246 (1999). A randomized, double-blinded, placebo-controlled, single center study of Compound (1) HCl hemihydrate salt Form A (hereinafter in this example simply Compound (1)) in healthy volunteers inoculated with live influenza A/Wisconsin/67/2005 (H3N2) challenge strain virus was conducted. Subjects received five daily doses of either placebo (N=33) or Compound (1) once a day (QD) (in capsule form consisting of neat Compound (1)): 100 mg (N=16), 400 mg (N=19), or 900 mg on Day 1 followed by 600 mg Days 2-5 (N=20), or 1200 mg on Day 1 followed by 600 mg Days 2-5 (N=18). Subjects underwent thrice daily nasal swabs, and kept thrice daily score cards for clinical symptoms from Days 1-7, and were discharged from the facility on Day 8, with safety follow-up at approximately Day 28. Nasal swabs were assayed for influenza virus in cell culture (primary analysis) and by qRT-PCR (secondary analysis).

Efficacy analyses were performed on the Full Analysis (FA) Set, defined as all randomized subjects who received at least one dose of study drug (Compound (1) or placebo) and whose viral concentrations were above or equal to the lower limit of quantification for the TCID₅₀ cell culture assay at any time point within 48 h post inoculation, or whose hemagglutination inhibition titer raised 4-fold or greater from baseline (Day 1) in the post inoculation period (N=74). The safety set included all subjects who were inoculated with influenza on Day 0 and who received at least one dose of either placebo or Compound (1) (N=104).

Efficacy Assessment

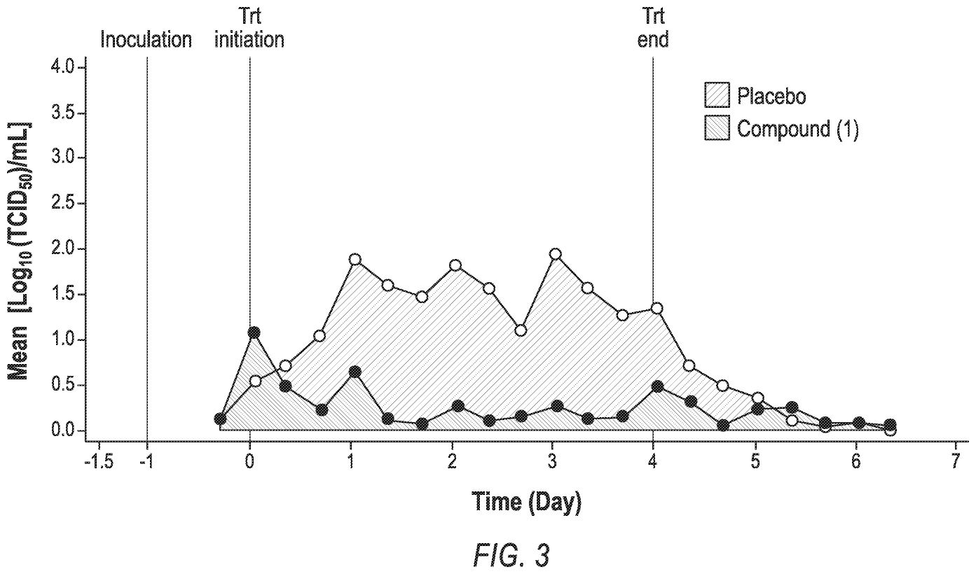

The primary measure in this study was the demonstration of a dose response trend in AUC of viral shedding between Days 1 (first day of drug dosing) through 7, as measured by TCID₅₀ in cell culture assay in the FA set. A statistically significant dose response trend was observed in median AUC viral shedding in nasal swabs (P=0.036, Jonckheere-Terpstra trend test). In addition, pairwise comparisons were performed between the pooled placebo group and each Compound (1) dose group for median AUC viral shedding, median duration of shedding, and mean magnitude of peak viral shedding (Table 32A). A statistically significant reduction in AUC viral shedding was observed for the 1200/600 mg dose group (P=0.010, Wilcoxon rank-sum test), and significant reductions in peak shedding were observed for the 1200/600 mg dose group (FIG. 3), the 400 mg dose group and the pooled Compound (1) dose groups. Additional FA group analyses were performed (data not shown).

Nasal influenza shedding was also quantified by qRT-PCR and results were similar to those observed with cell culture. There was no difference in rates of seroconversion between Compound (1) dose groups and placebo, as defined by a 4-fold or greater increase in anti-influenza titer from pre-inoculation baseline, suggesting that Compound (1) dosed 24 h after influenza inoculation did not affect the rate of acquisition of influenza infection and did not eliminate the subsequent humoral immune response to infection (Table 32A).

Subjects recorded clinical symptoms three times a day in diaries. An AUC of clinical and influenza-like symptom scores from Day 1 through Day 7 was calculated. Compared with placebo, the 1200/600 mg dose group of Compound (1) showed a statistically significant reduction in the median duration of composite clinical symptoms (P=0.001), the median AUC of influenza-like symptoms (P=0.040), and the median duration of influenza-like symptoms (P<0.001) (Table 32B).

TABLE 32A
Median AUC viral shedding, median duration of shedding,
and mean magnitude of peak viral shedding.

| | | Pooled | Compound (1) | | | | |
|---|---|---|---|---|---|---|---|
| | | Placebo | 100 mg | 400 mg | 900/600 mg | 1200/600 mg | Pooled |
| Endpoint [units] | | (N = 22) | (N = 12) | (N = 12) | (N = 14) | (N = 14) | (N = 52) |
| Viral | AUC, median | 5.85 | 1.25 | 0.70 | 3.20 | 0.35 | 0.65 |
| Shedding | (range) | | | | | | |
| by Tissue | [log₁₀ TCID₅₀ | (0.0, | (0.0, | (0.0, | (0.0, | (0.0, | (0.0, |
| Culture^{a} | mL*Day] | 17.1) | 16.1) | 18.0) | 16.1) | 8.4) | 18.0) |
| | P Value^{b} | NA | 0.269 | 0.206 | 0.723 | 0.010 | 0.057 |
| | Duration, | 2.38 | 0.96 | 1.60 | 2.71 | 0.00 | 0.71 |
| | median | | | | | | |
| | (95% CI)[Day] | (0.03, | (0.00, | (0.00, | (0.00, | (0.00, | (0.00, |
| | | 4.63) | 3.39) | NA) | 4.68) | 1.33) | 2.43) |
| | P Value^{d} | NA | 0.331 | 0.831 | 0.893 | 0.169 | 0.487 |
| | Peak, mean (SD) | 3.13 | 2.09 | 1.73 | 2.68 | 1.00 | 1.87 |
| | [log₁₀ | (1.878) | (2.209) | (1.976) | (2.201) | (1.365) | (2.002) |
| | TCID₅₀/mL] | | | | | | |
| | P Value^{c} | NA | 0.139 | 0.049 | 0.505 | 0.002 | 0.015 |
| Viral | AUC, median | 18.40 | 6.05 | 4.90 | 10.65 | 0.45 | 3.45 |
| Shedding | (range) | | | | | | |
| by qRT- | [log₁₀ copies/ | (0.0, | (0.0, | (0.0, | (0.0, | (0.0, | (0.0, |
| PCR^{e} | mL*Day] | 42.1) | 41.9) | 36.9) | 37.1) | 24.7) | 41.9) |
| | P Value^{b} | NA | 0.218 | 0.306 | 0.821 | 0.014 | 0.075 |
| | Duration, | 2.91 | 0.96 | 1.36 | 2.39 | 0.00 | 0.71 |
| | median | | | | | | |
| | (95% CI)[Day] | (0.03, | (0.00, | (0.00, | (0.00, | (0.00, | (0.00, |
| | | 5.35) | 3.39) | NA) | 5.01) | 0.66) | 2.394) |
| | P Value^{d} | NA | 0.318 | 0.753 | 0.602 | 0.084 | 0.238 |
| | Peak, mean (SD) | 5.36 | 4.36 | 3.90 | 5.08 | 2.37 | 3.91 |
| | [log₁₀ | (3.108) | (3.379) | (3.514) | (3.097) | (2.861) | (3.276) |
| | TCID₅₀/mL] | | | | | | |
| | P Value^{c} | NA | 0.380 | 0.202 | 0.794 | 0.007 | 0.081 |
| Serology^{f} | Sero- | 21/32 | 11/16 | 9/19 | 13/19 | 12/18 | 45/72 |
| | conversion, | | | | | | |
| | n/N (%) | (66%) | (69%) | (47%) | (68%) | (67%) | (63%) |
| | P Value | NA | >0.999 | 0.247 | >0.999 | >0.999 | 0.828 |

AUC: area under the value versus time curve; CI: confidence interval; NA: not applicable; qRT-PCR: quantitative reverse transcriptase polymerase chain reaction; SD: standard deviation; TCID50: 50% tissue culture infective dose.
Note:
Statistically significant P values (P < 0.05) are in bold font.
^{a}P = 0.036 for the dose response trend of AUC from Jonckheere-Terpstra trend test.
^{b}P value calculated from Wilcoxon rank-sum test.
^{c}P value calculated from ANOVA.
^{d}P value calculated from log-rank test.
^{e}P = 0.031 for the dose response trend of AUC from Jonckherre-Terpstra trend test.
^{f}Sero-conversion defined as ≥4-fold increase in anti-influenza antibody titer at Follow-up Visit compared with baseline. P value calculated using Fisher's Exact Test.

TABLE 32B
Median AUC, median duration, and mean magnitude of peak,
of composite clinical symptom and influenza like symptom.

| | | Pooled | Compound (1) | | | | |
|---|---|---|---|---|---|---|---|
| | | Placebo | 100 mg | 400 mg | 900/600 mg | 1200/600 mg | Pooled |
| Endpoint [units] | | (N = 22) | (N = 12) | (N = 12) | (N = 14) | (N = 14) | (N = 52) |
| Composite | AUC, median | 4.85 | 1.85 | 4.70 | 1.75 | 1.95 | 2.15 |
| Clinical | (range) | | | | | | |
| Symptom | [Grade*Day] | (0.0, | (0.0, | (0.0, | (0.0, | (0.0, | (0.0, |
| | | 23.5) | 25.3) | 16.0) | 32.3) | 5.5) | 32.3) |
| | P Value^{b} | NA | 0.422 | 0.694 | 0.595 | 0.83 | 0.211 |
| | Duration, | 3.69 | 3.21 | 3.34 | 2.69 | 1.88 | 2.34 |
| | median | | | | | | |
| | (95% CI)[Day] | (2.04, | (0.03, | (1.28, | (0.00, | (0.00, | (1.87, |
| | | 4.73) | 5.43) | 4.63) | 4.61) | 2.24) | 3.06) |
| | P Value^{d} | NA | 0.946 | 0.994 | 0.686 | 0.001 | 0.355 |
| | Peak, mean (SD) | 3.91 | 3.17 | 2.83 | 3.71 | 1.50 | 2.79 |
| | [Grade] | (3.637) | (3.881) | (2.167) | (4.232) | (1.286) | (3.158) |
| | P Value^{c} | NA | 0.532 | 0.366 | 0.863 | 0.036 | 0.187 |
| Influenza | AUC, median | 4.05 | 1.85 | 3.80 | 1.75 | 1.75 | 2.05 |
| like | (range) | | | | | | |
| Symptom | [Grade*Day] | (0.0, | (0.0, | (0.0, | (0.0, | (0.0, | (0.0, |
| | | 17.7) | 21.3) | 14.0) | 28.6) | 4.4) | 28.6) |
| | P Value^{b} | NA | 0.363 | 0.617 | 0.595 | 0.040 | 0.149 |
| | Duration, | 3.69 | 3.21 | 3.34 | 2.69 | 1.88 | 2.34 |
| | median | | | | | | |
| | (95% CI)[Day] | (2.04, | (0.00, | (1.28, | (0.00, | (0.00, | (1.87, |
| | | 4.73) | 5.40) | 4.63) | 4.61) | 2.24) | 3.00) |
| | P Value^{d} | NA | 0.957 | 0.994 | 0.653 | <0.001 | 0.342 |
| | Peak, mean (SD) | 3.41 | 2.75 | 2.42 | 3.21 | 1.36 | 2.42 |
| | [Grade] | (3.003) | (3.361) | (1.832) | (3.534) | (1.216) | (2.689) |
| | P Value^{c} | NA | 0.511 | 0.323 | 0.838 | 0.034 | 0.168 |

AUC: area under the value versus time curve; CI: confidence interval; NA: not applicable.
Note:
Statistically significant P values (P < 0.05) are in bold font.
^{b}P value calculated from Wilcoxon rank-sum test.
^{c}P value calculated from ANOVA.
^{d}P value calculated from log-rank test.

Safety Assessment

Compound (1) was well tolerated, and there were no discontinuations due to Compound (1)-related adverse events (AE) nor were there any serious adverse events. A list of adverse events occurring in ≥10% of subjects in any treatment group is presented (Table 33). Influenza-like illness was the most frequently reported adverse event, and was reported by an approximately equal proportion of subjects in the placebo and Compound (1) groups. Adverse events that occurred with ≥10% difference in incidence between the Compound (1) groups and the placebo recipients were decreased blood phosphorus level (18.1%, Compound (1); 0%, placebo), rhinorrhea (Compound (1), 4.2%; 18.8%, placebo), and nasal congestion (1.4%, Compound (1); 15.6% placebo). In addition, elevations in alanine aminotransferase (ALT) were observed in both placebo and Compound (1) recipients. Neither liver function abnormalities nor serum phosphate decreases were observed in the first-in-human dose escalation study of Compound (1) at single doses up to 1600 mg and multiple doses up to 800 mg daily for 10 days; both elevations in ALT and decreases in serum phosphate have been previously reported with upper respiratory viral infections.

TABLE 33
A list of adverse events occurring in ≥10% of subjects in any treatment group.

| | Pooled | | Compound (1) | | | | | | | | | |
|---|---|---|---|---|---|---|---|---|---|---|---|---|
| | Placebo | | 100 mg | | 400 mg | | 900/600 mg^{a} | | 1200/600 mg^{b} | | Pooled | |
| | N = 32 | | N = 16 | | N = 19 | | N = 19 | | N = 18 | | N = 72 | |
| Preferred Term | n(%) | | n(%) | | n(%) | | n(%) | | n(%) | | n(%) | |
| Influenza-like | 12 | (37.5) | 8 | (50.0) | 10 | (52.6) | 9 | (47.4) | 7 | (38.9) | 34 | (47.2) |
| illness^{c} | | | | | | | | | | | | |

| | | | | | | | | | | | |
|---|---|---|---|---|---|---|---|---|---|---|---|
| Alanine | 5 | (15.6) | 3 | (18.8) | 1 | (5.3) | 0 | 6 | (33.3) | 10 | (13.9) |
| aminotransferase | | | | | | | | | | | |
| increased | | | | | | | | | | | |

| | | | | | | | | | | |
|---|---|---|---|---|---|---|---|---|---|---|
| Blood | 0 | 3 | (18.8) | 0 | 6 | (31.6) | 4 | (22.2) | 13 | (18.1) |
| phosphorus | | | | | | | | | | |
| decreased | | | | | | | | | | |

| Spirometry | 2 | (6.3) | 2 | (12.5) | 4 | (21.1) | 0 | 4 | (22.2) | 10 | (13.9) |
|---|---|---|---|---|---|---|---|---|---|---|---|
| abnormal | | | | | | | | | | | |
| Rhinorrhea | 6 | (18.8) | 0 | | 2 | (10.5) | 0 | 1 | (5.6) | 3 | (4.2) |
| Headache | 2 | (6.3) | 1 | (6.3) | 4 | (21.1) | 0 | 2 | (11.1) | 7 | (9.7) |
| Dermatitis | 3 | (9.4) | 3 | (18.8) | 0 | | 0 | 0 | | 3 | (4.2) |
| contact | | | | | | | | | | | |
| Nasal congestion | 5 | (15.6) | 0 | | 0 | | 0 | 1 | (5.6) | 1 | (1-4) |
| Aspartate | 1 | (3.1) | 1 | (6.3) | 1 | (5.3) | 0 | 2 | (11.1) | 4 | (5.6) |
| aminotransferase | | | | | | | | | | | |
| increased | | | | | | | | | | | |

| | | | | | | | | | | |
|---|---|---|---|---|---|---|---|---|---|---|
| Oropharylngeal | 1 | (3.1) | 2 | (12.5) | 0 | 1 | (5.3) | 0 | 3 | (4.2) |
| pain | | | | | | | | | | |

| | | | | | | | | | | |
|---|---|---|---|---|---|---|---|---|---|---|
| Tension | 1 | (3.1) | 0 | 2 | (10.5) | 1 | (5.3) | 0 | 3 | (4.2) |
| Headache | | | | | | | | | | |

| | | | | | | | | | |
|---|---|---|---|---|---|---|---|---|---|
| Malaise | 1 | (3.1) | 2 | (12.5) | 0 | 0 | 0 | 2 | (2.8) |

| | | | | | | | | | |
|---|---|---|---|---|---|---|---|---|---|
| Nausea | 0 | 0 | 2 | (10.5) | 1 | (5.3) | 0 | 3 | (4.2) |

Notes:
A subject with multiple events was counted once under the AE. Subjects may appear in multiple categories.
^{a}Single loading dose of 900 mg on Day 1and 600 mg qd on Days 2 through 5.
^{b}Single loading dose of 1200 mg on Day 1 and 600 mg qd on Days 2 through 5.
^{c}Influenza-like illness, as defined in the efficacy analysis, was assessed based on the parameters listed in the text. The AE of influenza-like illness was determined by physician.

### DISCUSSION

In an influenza challenge study in healthy volunteers, Compound (1) demonstrated a dose response trend in AUC viral titer in nasal swabs by both TCID₅₀ cell culture and qRT-PCR, and the highest dose of Compound (1) evaluated caused a significant reduction in AUC viral titer as well as in AUC and duration of influenza symptoms. Although, a similar magnitude of improvement over placebo was not observed in the second highest dose group, 900/600 mg (Table 27), this dose did demonstrate similar results to the 1200/600 mg dose with respect to median AUC for composite clinical symptom and influenza-like symptom endpoints (Table 28); the reasons for this discrepancy are not completely understood. While no definite safety trends were encountered in the POC trial, the phosphate decreases and ALT elevations observed suggest that appropriate monitoring of both parameters will need to be employed in future studies.

Overall, the limitations of the influenza challenge model are that the influenza virus utilized in this study is a strain that has been specifically selected so as not to produce the most severe clinical symptoms of influenza virus infection. In addition, the viral inoculum administered is likely larger than the inoculum in natural influenza exposure. The timing of Compound (1) dosing 24 h after exposure may not be a realistic timeframe for initiation of therapy in the community setting in which patients do not often seek diagnosis or treatment until they have developed substantial symptoms, likely more than 24 h after exposure. However, given that naturally infected subjects are initially inoculated with a much lower viral titer the time scales are not directly comparable.

In summary, Compound (1) is a potent influenza A PB2 inhibitor that represents a distinct and novel class of antiviral agent. The properties of this inhibitor, as described by both the preclinical and clinical data, indicate that Compound (1) is an exciting candidate for further evaluation with several potential advantages over current antiviral agents used to treat influenza infection.

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-171. (canceled)

172. A pharmaceutical composition comprising
a) a plurality of granules forming an intragranular phase of the composition, wherein the intragranular phase comprises
i) a crystalline HCl salt of Compound (1).½H₂O wherein Compound (1) is represented by the following structural formula: 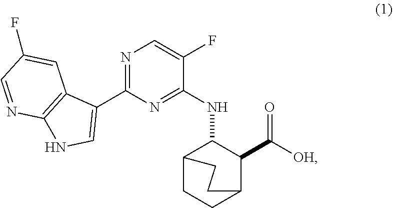
and
ii) one or more excipients selected from a first disintegrant, a binder, and a wetting agent; and
b) an extragranular phase of the composition comprising a diluent, a second disintegrant, a glidant, and a lubricant,
wherein the HCl salt of Compound (1).½H₂O has a concentration of 45 wt % to 55 wt %, the combined concentration of the excipients is 45 wt % to 55 wt %, and each wt % is by weight of the pharmaceutical composition.

173. The pharmaceutical composition of claim 172, comprising 1.45 wt % to 1.62 wt % of the first disintegrant, wherein each wt % is by weight of the pharmaceutical composition.

174. The pharmaceutical composition of claim 172, wherein the first disintegrant comprises croscarmellose sodium, crospovidone, or a combination thereof.

175. The pharmaceutical composition of claim 172, comprising 1.5 wt % to 2.5 wt % of the binder, wherein each wt % is by weight of the pharmaceutical composition.

176. The pharmaceutical composition of claim 172, wherein the binder comprises hydroxypropyl methylcellulose.

177. The pharmaceutical composition of claim 172, comprising 0.35 wt % to 0.65 wt % of the wetting agent, wherein each wt % is by weight of the pharmaceutical composition.

178. The pharmaceutical composition of claim 172, wherein the wetting agent comprises polysorbate 20.

179. The pharmaceutical composition of claim 172, comprising 25 wt % to 40 wt % of the diluent, wherein each wt % is by weight of the pharmaceutical composition.

180. The pharmaceutical composition of claim 172, wherein the diluent comprises silicified microcrystalline cellulose, microcrystalline cellulose, starch, or any combination thereof.

181. The pharmaceutical composition of claim 180, wherein the diluent comprises 4.85 wt % to 5.25 wt % of microcrystalline cellulose, 22 wt % to 24 wt % of silicified microcrystalline cellulose, and 4.5 wt % to 6.5 wt % of partially or fully pregelatinized maize starch, wherein each wt % is by weight of the pharmaceutical composition.

182. The pharmaceutical composition of claim 172, comprising 0.5 wt % to 1.5 wt % of the glidant, wherein each wt % is by weight of the pharmaceutical composition.

183. The pharmaceutical composition of claim 172, wherein the glidant comprises silicon dioxide.

184. The pharmaceutical composition of claim 183, wherein the glidant comprises colloidal anhydrous silica.

185. The pharmaceutical composition of claim 172, comprising 5 wt % to 6 wt % of the second disintegrant, wherein each wt % is by weight of the pharmaceutical composition.

186. The pharmaceutical composition of claim 172, wherein the second disintegrant comprises croscarmellose sodium, crospovidone, or a combination thereof.

187. The pharmaceutical composition of claim 172, comprising 4.75 wt % to 5.25 wt % of the lubricant, wherein each wt % is by weight of the pharmaceutical composition.

188. The pharmaceutical composition of claim 172, wherein the lubricant comprises sodium stearyl fumarate, magnesium stearate, or a combination thereof.

189. The pharmaceutical composition of claim 188, wherein the lubricant comprises sodium stearyl fumarate.

190. The pharmaceutical composition of claim 172, wherein the first disintegrant and the second disintegrant each comprise crospovidone.

191. The pharmaceutical composition of claim 172, wherein the composition is a coated or uncoated tablet comprising the intragranular phase and the extragranular phase.

192. The pharmaceutical composition of claim 172, comprising 47.5 wt % to 52.5 wt % of the crystalline HCl salt of Compound (1).½H₂O wherein each wt % is by weight of the pharmaceutical composition

193. A pharmaceutical composition comprising
a) a plurality of granules forming an intragranular phase of the composition, wherein the intragranular phase comprises
i) 47.5 wt % to 52.5 wt % of a crystalline HCl salt of Compound (1).½H₂O wherein Compound (1) is represented by the following structural formula: 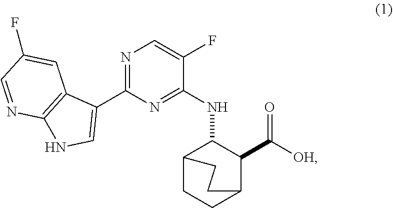
ii) 1.45 wt % to 1.62 wt % of a first disintegrant comprising crospovidone, and
iii) 1.5 wt % to 2 wt % of a binder comprising hydroxypropyl methylcellulose; and
b) an extragranular phase comprising
i) 25 wt % to 40 wt % of a diluent comprising silicified microcrystalline cellulose, microcrystalline cellulose, partially or fully pregelatinized maize starch, or any combination thereof,
ii) 0.5 wt % to 1.5 wt % of a glidant comprising silicon dioxide,
iii) 5 wt % to 6 wt % of a second disintegrant comprising crospovidone, and
iv) 4.75 wt % to 5.25 wt % of a lubricant comprising sodium stearyl fumarate,
wherein the composition is a coated or uncoated tablet comprising the intragranular phase and the extragranular phase and each wt % is by weight of the pharmaceutical composition.

194. A process for producing a pharmaceutical composition according to claim 172, comprising:
a. mixing a binder and a wetting agent in water to form a substantially clear binder solution;
b. mixing crystalline HCl salt of Compound (1).½H₂O and a first disintegrant under heating conditions in a fluid bed granulizer to form a substantially homogenous mixture;
c. spraying the binder solution onto the homogenous mixture to form wet granules;
d. drying the wet granules to form dry granules;
e. mixing the dry granules and a glidant to form a substantially homogenous second mixture;
f. mixing a diluent, a second disintegrant, and the homogenous second mixture to form a substantially homogenous third mixture;
g. mixing a lubricant and the homogenous third mixture to form a substantially homogenous fourth mixture; and
h. compressing the homogenous fourth mixture into tablets using a tablet press,
wherein the wet granules and dry granules are formed under fluidizing conditions.

195. A method of treating influenza in a subject, comprising administering to the subject a therapeutically effective amount of a pharmaceutical composition according to claim 172.

196. A dosage regimen comprising administering to a subject an effective amount of a pharmaceutical composition according to claim 172 in a dosage amount of 250 mg to 350 mg of crystalline HCl salt of Compound (1).½ H₂O twice per day.
